(19) United States
(12) Patent Application Publication
(10) Pub. No.: US 2024/0116948 A1
(43) Pub. Date: Apr. 11, 2024

(54) PYRIDO OXAZINE DERIVATIVES AS ALK5 INHIBITORS

(71) Applicant: CHIESI FARMACEUTICI S.P.A., Parma (IT)

(72) Inventors: Daniele PALA, Parma (IT); Daniela PIZZIRANI, Parma (IT); Paolo BRUNO, Parma (IT); Matteo BIAGETTI, Parma (IT); Paolo RONCHI, Parma (IT); Sara GUARIENTO, Parma (IT); Barbara BERTANI, Parma (IT); Donatella RESCIGNO, Parma (IT)

(73) Assignee: CHIESI FARMACEUTICI S.P.A., Parma (IT)

(21) Appl. No.: 18/269,042

(22) PCT Filed: Dec. 20, 2021
(86) PCT No.: PCT/EP2021/086707

§ 371 Date: Jun. 22, 2023

(30) Foreign Application Priority Data
Dec. 23, 2020 (EP) 20217161.7

(51) Int. Cl. C07D 498/04; C07D 519/00

(52) U.S. Cl. CPC C07D 498/04; C07D 519/00

(57) ABSTRACT

The present invention relates to a compound of general formula (I) inhibiting the transforming growth factor-β (TGF-β) type I receptor (ALK5), methods of preparing such compounds, pharmaceutical compositions containing them and therapeutic use thereof. The compounds of the invention may be useful in the treatment of diseases or conditions associated with a dysregulation of ALK5 signaling pathway in a mammal.

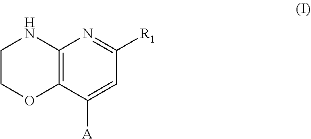

### FIELD OF THE INVENTION

The present invention relates to compounds inhibiting the transforming growth factor β (TGF β) type I receptor (ALK5) (hereinafter ALK5 inhibitors), methods of preparing such compounds, pharmaceutical compositions containing them and therapeutic use thereof. The compounds of the invention may be useful in the treatment of many diseases, disorders, or conditions associated with ALK5 signaling pathway.

### BACKGROUND OF THE INVENTION

The Transforming Growth Factor β (TGF β) is a protein belonging to the TGF R superfamily.

It is involved in several processes, both cellular, such as proliferation, migration and differentiation, and biological, including wound healing, immunesuppression, cancerogenesis and extracellular matrix production.

The TGF β superfamily also includes, among others, other members known as activins (Acts) (see e.g. Hinck A P, FEBS Letters 586 (2012); 1860-1870).

The binding of the peptide initiates the TGF β signaling cascade through the formation of a heterotetrameric complex composed of two different serine/threonine kinases receptors: type 1 (TGFβR1/ALK5) and type 2 (TGFβR2).

TGFβR1/ALK5 is recruited and activated through the phosphorylation of its intracellular domain by TGFβR2, leading in turn to the phosphorylation of the receptor-activated (R)-Smad family, resulting in the activation of target gene transcription (see e.g. Sheppard D., Proc Am Thorac Soc. (2006); (3):413-417).

Similarly to the TGF β signaling, the type I receptor for activin, ALK4, leads to the activation of target gene transcription (see e.g. Heldin C H et al., Cold Spring Harb Perspect Biol. (2016) Aug. 1; 8(8)).

Several studies have linked an excessive and/or dysregulated TGFβ activity with many diseases including cancer and fibrosis (see e.g. Syed V, J Cell Biochem. (2016) June; 117(6):1279-87; Jakowlew S B. Cancer Metastasis Rev. (2006) September; 25(3):435-57). Among fibrotic disorders, a crucial role of TGFβ has been shown in organs such as lung, heart, liver, and kidney (see e.g. Alhamad E H, J Thorac Dis. (2015); 7(3):386-93). In particular, TGFβ expression is increased in fibrotic lung diseases, such as idiopathic pulmonary fibrosis (IPF), and in chronic inflammatory conditions, such as chronic obstructive pulmonary disease and asthma (see e.g. Thomas B J et al., Am J Respir Cell Mol Biol. (2016); (55):759-766).

In lung, TGFβ is expressed in several cell types, like epithelial cells, endothelial cells, connective tissue cells, macrophages and fibroblasts.

These cell populations may produce excess of TGFβ in IPF human lung tissue. Moreover, high levels of TGFβ have been detected in lung tissue and BAL of IPF patients (see e.g. Bergeron A et al., Eur Respir J (2003); 22:69-76).

TGFβ gene expression and TGFβ protein production have been observed to increase in a variety of animal models of pulmonary fibrosis caused by bleomycin, silica, asbestos, and radiation (see e.g. Wei F et al., Int Immunopharmacol. (2017) July; 48:67-75; Choe J Y et al., Inflamm Res. (2010) March; 59(3):177-88; Wang X et al., Respir Res (2009); 10, 36) and it has also been reported how the TGFβ expression is sufficient to induce progressive fibrosis in rodents (see e.g. Sime P J et al., J Clin Invest (1997); 100:768-776; Kim K K et al.).

Contrarily, TGFβ signaling inhibition obtained by employing knockout (KO) animals can inhibit fibrosis development through TGFβ-linked mechanisms (see e.g. Bonniaud P et al., Am J Respir Crit Care Med (2005); 171:889-898; 34).

Similar results have been achieved with inhibition of TGFβ R1 in mouse bleomycin disease model (see e.g. Wei Y et al., J Clin Invest. (2017); 127(10):3675-3688).

Activin signaling dysregulation, similarly to TGFβ, is associated to fibroblasts proliferation, myofibroblasts differentiation and accumulation of extracellular matrix (ECM) (see e.g. Yamashita et al., J. Am. Soc. Nephrol. (2004) 15, 91-101). Moreover, overexpression of activin has been linked to pathological conditions and fibrosis development in different organs, such as liver (see e.g. Patella et al., Am. J. Physiol. Gastrointest. Liver Physiol. (2006) 290, G137-G144), kidney (see e.g. Agapova et al., Kidney Int. (2016) 89, 1231-1243), heart (see e.g. Yndestad et al., Circulation (2004) 109, 1379-1385), and lung (see e.g. de Kretser et al., Crit. Care (2013) 17:R263).

Taken together these data suggest the importance of targeting ALK5 receptor to treat pharmacologically the aforementioned diseases, linked to dysregulated TGF signaling pathway.

The TGFβ signaling is strongly involved in the cardiovascular homeostasis (see e.g. van Meeteren L A et al., Springer (2013)). Several studies in humans and mice have shown the main role of TGFβ in angiogenesis and vascular morphogenesis. Moreover, TGFβ plays a key role in the development and functionality of cardiac valves. It is therefore clear the importance of a selective regulation of TGFβ pathway to target the pathological effects avoiding the suppression of the signaling needed for a correct homeostasis.

The answer to this crucial point could be addressed by using the inhalation route to deliver an antiTGFβ drug.

The inhalatory route would allow the treatment of the affected lung compartment bypassing the issue of the heart exposure.

Various compounds have been described in the literature as ALK5 and/or ALK4 inhibitors.

WO2008/006583, WO2009/087212, WO2009/087224, WO2009/087225, WO2009/133070, WO2009/013335 and WO2009/050183 (Novartis) disclose respectively pyrimidine, pyridine, imidazo pyridine, pyrrolo pyrimidine and pyrrolo pyridine, imidazo pyridazine, imidazo pyridine derivatives for the treatment of ALK4 or ALK5 mediated diseases useful for the treatment of inflammatory or obstructive airways diseases, pulmonary hypertension and pulmonary fibrosis.

WO00/61576 and US2003/0149277 (Smithkline Beecham Corp) disclose triarylimidazole derivatives as ALK5 inhibitors useful for the treatment of, among others, renal disease, wound healing, kidney disease, congestive heart failure, ulcers, impaired neurological function and any disease wherein fibrosis is a major component.

Wo01/62756 (Smithkline Beecham P.L.C.) discloses pyridinylimidazole derivatives as ALK5 inhibitors useful for the treatment of, among others, renal disease, wound healing, kidney disease, congestive heart failure, ulcers, impaired neurological function and any disease wherein fibrosis is a major component.

WO03/087304 (Biogen Inc.) discloses tri-substituted heteroaryls as ALK5 and/or ALK4 inhibitors useful for the treatment of, among others, idiopathic pulmonary fibrosis, diabetic nephropathy, hepatic fibrosis, pulmonary fibrosis, acute lung injury, post-infarction cardiac fibrosis, fibrotic cancers and fibroma.

WO2013/009140 (SK Chemicals Co) discloses 2-pyridyl substituted imidazole derivatives as ALK5 and/or ALK4 receptors useful for the treatment of, among others, renal-, liver- or pulmonary fibrosis.

WO2018/215668 (Glenmark) discloses, among other compounds, pyrido oxazine amino derivatives as inhibitors of MAP4K1, wherein the amino group is linked to a substituted aryl ring. These compounds are disclosed as useful for the treatment of autoimmune, neurodegenerative, neurological, inflammatory, hyperproliferative and cardiovascular diseases.

Pyrido oxazine derivatives linked to a pyridinyl ring or to a pyridinyl ring fused to a 5 or 6-membered heteroaryl ring have not been disclosed in the literature.

Of note, inhibition of ALK5 receptor may be useful for the treatment of fibrosis and diseases, disorders and conditions that result from fibrosis.

Several efforts have been done in the past years to develop novel ALK5 receptor inhibitors useful for the treatment of several diseases and some of those compounds have shown efficacy also in humans.

However, there remains a potential for developing inhibitors of receptors ALK5 characterized by good potency, useful for the treatment of diseases or conditions associated with a dysregulation of ALK5 signaling pathway, in particular fibrosis.

In particular, there remains a potential for developing inhibitors of receptor ALK5 useful for the treatment of diseases or conditions associated with a dysregulation of ALK5 signaling in the respiratory field, in particular idiopathic pulmonary fibrosis (IPF), to be administered by the inhalation route and characterized by a good inhalatory profile, that corresponds to a good activity in the lung, a good lung retention and to a low metabolic stability in order to minimize the systemic exposure and correlated safety issues.

In this direction, we have surprisingly found a new series of compounds of general formula (I) that solves the problem of providing potent inhibitors of ALK5 receptor for administration by inhalation, that shows, at the same time, a good inhalatory profile, low metabolic stability, low systemic exposure, improved safety and tolerability.

### SUMMARY OF THE INVENTION

In a first aspect the present invention relates to compounds of formula (I)

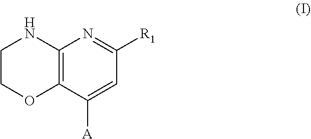

wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

A is selected from the group consisting of A₁, A₂, A₃, A₄ and A₅

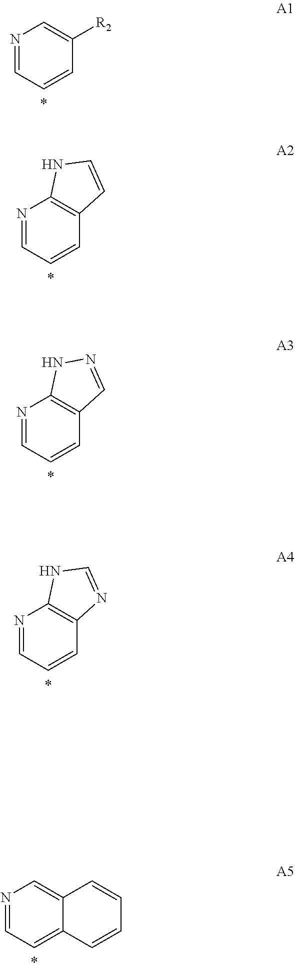

R₂ is H or is selected from the group consisting of —NR₃C(O)R₄, —C(O)NR₃R₄, —C(O)OR₅, NR₃R₄ and —OR₆;

R₃ is H or —(C₁-C₆)alkyl;

R₄ is H or is selected from the group consisting of —(C₁-C₆)alkylene-NR_{A}R_{B} and —(C₁-C₆)alkylene-heterocycloalkyl, wherein said heterocycloalkyl is optionally substituted by one or more groups selected from oxo and —(C₁-C₆)alkyl;

R₅ is selected from the group consisting of —(C₁-C₆)alkyl, —(C₁-C₆)alkylene-NR_{A}R_{B} and cycloalkyl;

R₆ is H or is selected from the group consisting of —(C₁-C₆)alkyl, heterocycloalkyl, —(C₁-C₆)alkylene-NR_{A}R_{B}, —(C₁-C₆)alkylene-heterocycloalkyl, wherein said heterocycloalkyl is optionally substituted by one or more —(C₁-C₆)alkyl;

R_{A} is H or is selected from the group consisting of —(C₁-C₆)alkyl and —(C₁-C₆)hydroxyalkyl;

R_{B} is selected from the group consisting of —(C₁-C₆)alkyl, heterocycloalkyl, —(C₁-C₆)alkylene-C(O)O—(C₁-C₆)alkyl and —(C₁-C₆)hydroxyalkyl; and pharmaceutically acceptable salts thereof.

In a second aspect, the invention refers to a pharmaceutical composition comprising a compound of formula (I) and pharmaceutically acceptable salts thereof in admixture with one or more pharmaceutically acceptable carrier or excipient.

In a third aspect, the invention refers to a compound of formula (I) and pharmaceutically acceptable salts or to a pharmaceutical composition comprising a compound of formula (I) and pharmaceutically acceptable salts thereof for use as a medicament.

In a further aspect, the invention refers to a compound of formula (I) and pharmaceutically acceptable salts thereof or to a pharmaceutical composition comprising a compound of formula (I) and pharmaceutically acceptable salts thereof for use in preventing and/or treating a disease, disorder or condition mediated by ALK5 signaling pathway in a mammal.

In a further aspect, the invention refers to a compound of formula (I) and pharmaceutically acceptable salts thereof or to a pharmaceutical composition comprising a compound of formula (I) and pharmaceutically acceptable salts thereof for use in the prevention and/or treatment of fibrosis and/or diseases, disorders, or conditions that involve fibrosis.

In a further aspect, the invention refers to a compound of formula (I) and pharmaceutically acceptable salts thereof or to a pharmaceutical composition comprising a compound of formula (I) and pharmaceutically acceptable salts thereof for use in the prevention and/or treatment idiopathic pulmonary fibrosis (IPF).

### DETAILED DESCRIPTION OF THE INVENTION

### Definitions

Unless otherwise specified, the compound of formula (I) of the present invention is intended to include also tautomer or pharmaceutically acceptable salt or solvate thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “(Cₓ-C_{y})alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})alkylene” wherein x and y are integers, refers to a Cₓ-C_{y}alkyl radical having in total two unsatisfied valencies, such as a divalent methylene radical.

The term “(Cₓ-C_{y})cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono cyclic carbon ring systems which have 6 ring atoms wherein the ring is aromatic. Examples of suitable aryl monocyclic ring systems include, for instance, phenyl.

The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond.

The term “(Cₓ-C_{y})heterocycloalkyl” wherein x and y are integers, refers to saturated or partially unsaturated monocyclic (Cₓ-C_{y})cycloalkyl groups in which at least one ring carbon atom is replaced by at least one heteroatom (e.g. N, S or O) or may bear an -oxo (═O) substituent group. Said heterocycloalkyl may be further optionally substituted on the available positions in the ring, namely on a carbon atom, or on an heteroatom available for substitution. Substitution on a carbon atom includes spiro disubstitution as well as substitution on two adjacent carbon atoms, in both cases thus form additional condensed 5 to 6 membered heterocyclic ring.

The term “(Cₓ-C_{y})hydroxyalkyl” wherein x and y are integers, refers to the above defined “(C₁-C₆)alkyl” groups wherein one or more hydrogen atoms are replaced by one or more hydroxy (OH) group.

Throughout the specification the use of an asterisk “*” in the definition of a structural formula, indicates the point of attachment for the radical group to the rest of the molecule.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O). The present invention relates to novel compounds differing from the structures disclosed in the art at least for a common new core scaffold. In fact the invention relates to compounds that are [2H,3H,4H-pyrido[3,2-b][1,4]oxazin-8-yl] derivatives wherein a carbon atom of the the pyrido oxazine bicycle is linked to the carbon atom of a pyridinyl or of a pyridinyl fused to a 5 or 6-membered heterocyclic ring, which are inhibitors of receptor ALK5, that have therapeutically desirable characteristics, particularly promising for some fibrosis, including idiopathic pulmonary fibrosis (IPF).

The compounds of the invention are active as inhibitors of ALK5 receptor, they are potent and show improved properties such as a good inhalatory profile, a low metabolic stability, a low systemic exposure, improved safety and tolerability.

In this respect, the state of the art does not describe or suggest pyrido oxazine derivatives of general formula (I) of the present invention having inhibitory activity on receptor ALK5 which represents a solution to the aforementioned need.

In more details, the present invention refers to a series of compounds represented by the general formula (I) as herein below described in details, which are endowed with an inhibitory activity on receptor ALK5.

Advantageously, the inhibitory action on receptor can be effective in the treatment of those diseases where these receptors play a relevant role in the pathogenesis such as fibrosis and disease, disorder and condition from fibrosis.

Differently from similar compounds of the prior art, the compounds of formula (I) of the present invention are able to act as inhibitors of ALK5 receptor, particularly appreciated by the skilled person when looking at a suitable and efficacious compounds useful for the treatment of fibrosis, in particular idiopatic pulmonary fibrosis.

As indicated in the experimental part, in particular in Table 12, the compounds of formula (I) of the present invention show a notable potency with respect to their inhibitory activity on receptor ALK5, below about 10 nM, confirming that they are able to inhibit ALK5 receptor involved in fibrosis and diseases that result from fibrosis.

As indicated in the experimental part, comparative examples, in particular in Table 13, it is shown that, conversely to the compounds C1 and C2 characterized by a phenyl ring linked to the pyrido oxazine bicycle, in the compounds of the present invention, characterized by a pyridinyl ring or a pyridinyl ring fused to a 5 or 6-membered heteroaryl ring linked to the pyrido oxazine bicycle, the presence of the pyridinyl ring or of a pyridinyl ring fused to a 5 or 6-membered heteroaryl ring, unexpectedly and remarkably determines a relevant increase in the inhibitory activity on the ALK5 receptor.

Advantageously, the compounds of the present invention are endowed with very high potency, they could be administered in human at a lower dosage respect to the compounds of the prior art, thus reducing the adverse events that typically occur administering higher dosages of drug.

In addition to being notably potent with respect to their inhibitory activity on receptor ALK5, the compounds of the present invention are also characterized by a good inhalatory profile, that permits to act effectively on the lung compartment and have, at the same time, a low metabolic stability, that allows to minimize the drawbacks associated with the systemic exposure, such as safety and tolerability issues.

Therefore, the compounds of the present invention are particularly appreciated by the skilled person when looking at a suitable and efficacious compounds useful for the treatment of fibrosis, in particular idiopatic pulmonary fibrosis, administered by the inhalation route and characterized by a good inhalatory profile, that corresponds to a good activity on the lung, a good lung retention and to a low metabolic stability, that minimizes the systemic exposure and correlated safety issues.

Thus, in one aspect the present invention relates to a compound of general formula (I) as ALK5 inhibitor

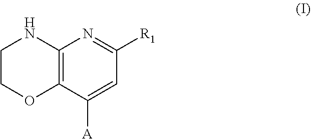

wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

A is selected from the group consisting of A₁, A₂, A₃, A₄ and A₅

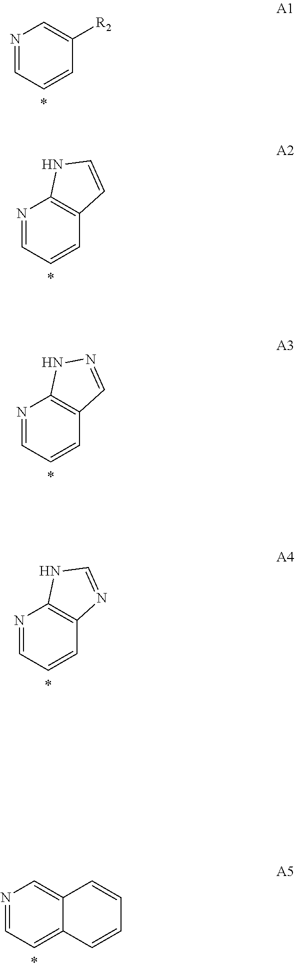

R₂ is H or is selected from the group consisting of —NR₃C(O)R₄, —C(O)NR₃R₄, —C(O)OR₅, NR₃R₄ and —OR₆;

R₃ is H or —(C₁-C₆)alkyl;

R₄ is H or is selected from the group consisting of —(C₁-C₆)alkylene-NR_{A}R_{B} and —(C₁-C₆)alkylene-heterocycloalkyl, wherein said heterocycloalkyl is optionally substituted by one or more groups selected from oxo and —(C₁-C₆)alkyl;

R₅ is selected from the group consisting of —(C₁-C₆)alkyl, —(C₁-C₆)alkylene-NR_{A}R_{B} and cycloalkyl;

R₆ is H or is selected from the group consisting of —(C₁-C₆)alkyl, heterocycloalkyl, —(C₁-C₆)alkylene-NR_{A}R_{B}, —(C₁-C₆)alkylene-heterocycloalkyl, wherein said heterocycloalkyl is optionally substituted by one or more —(C₁-C₆)alkyl;

R_{A} is H or is selected from the group consisting of —(C₁-C₆)alkyl and —(C₁-C₆)hydroxyalkyl;

R_{B} is selected from the group consisting of —(C₁-C₆)alkyl, heterocycloalkyl, —(C₁-C₆)alkylene-C(O)O—(C₁-C₆)alkyl and —(C₁-C₆)hydroxyalkyl;

and pharmaceutically acceptable salts thereof.

In a particularly preferred embodiment the present invention refers to a compound of formula (I), wherein A is group A1

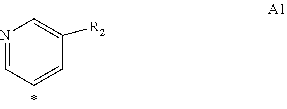

represented by the formula (Ia)

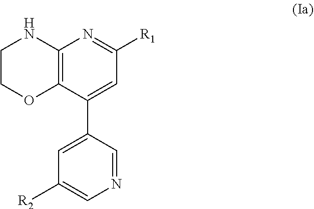

wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is H or is selected from the group consisting of —NR₃C(O)R₄, —C(O)NR₃R₄, —C(O)OR₅, NR₃R₄ and —OR₆;

R₃ is H or —(C₁-C₆)alkyl;

R₄ is H or is selected from the group consisting of —(C₁-C₆)alkylene-NR_{A}R_{B} and —(C₁-C₆)alkylene-heterocycloalkyl, wherein said heterocycloalkyl is optionally substituted by one or more groups selected from oxo and —(C₁-C₆)alkyl;

R₅ is selected from the group consisting of —(C₁-C₆)alkyl, —(C₁-C₆)alkylene-NR_{A}R_{B} and cycloalkyl;

R₆ is H or is selected from the group consisting of —(C₁-C₆)alkyl, heterocycloalkyl, —(C₁-C₆)alkylene-NR_{A}R_{B}, —(C₁-C₆)alkylene-heterocycloalkyl, wherein said heterocycloalkyl is optionally substituted by one or more —(C₁-C₆)alkyl;

R_{A} is H or is selected from the group consisting of —(C₁-C₆)alkyl and —(C₁-C₆)hydroxyalkyl;

R_{B} is selected from the group consisting of —(C₁-C₆)alkyl, —(C₁-C₆)alkylene-C(O)O—(C₁-C₆)alkyl and —(C₁-C₆)hydroxyalkyl;

and pharmaceutically acceptable salts thereof.

According to a preferred embodiment, the invention refers to at least one of the compounds of Formula (Ia) listed in the Table 1 below and pharmaceutically acceptable salts thereof.

TABLE 1
List of preferred compounds of Formula (Ia)

| Example No. | Structure | Chemical Name |
|---|---|---|
| 1 | 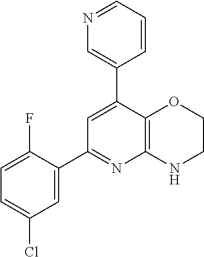 [SMILES: Fc1ccc(Cl)cc1-c1cc(-c2cccnc2)c2c(n1)NCCO2] | 3-[6-(5-chloro-2-fluorophenyl)- 2H,3H,4H-pyrido[3,2-b][1,4]oxazin- 8-yl]pyridine |
| 5 | 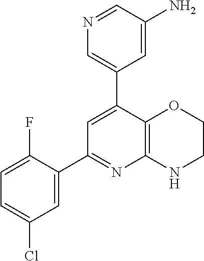 [SMILES: Nc1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]pyridin-3-amine |
| 6 | 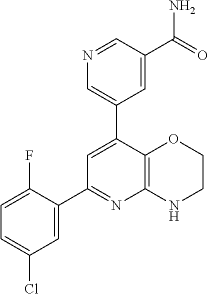 [SMILES: NC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]pyridine-3-carboxamide |
| 7 | 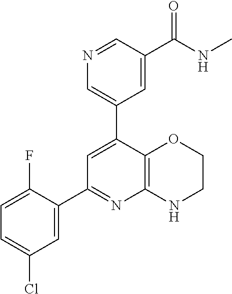 [SMILES: CNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-methylpyridine-3- carboxamide |
| 8 | 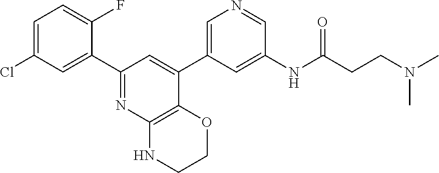 [SMILES: CN(C)CCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-2-yl}-3- (dimethylamino)propanamide |
| 9 | 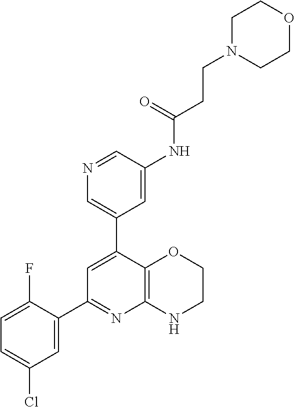 [SMILES: O=C(CCN1CCOCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(morpholin-4- yl)propanamide |
| 11 | 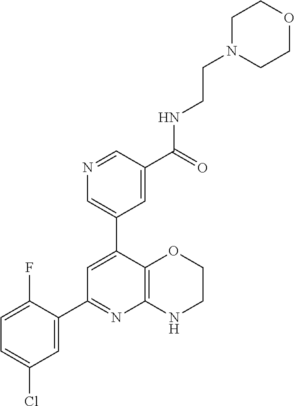 [SMILES: O=C(NCCN1CCOCC1)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[2-(morpholin-4- yl)ethyl]pyridine-3-carboxamide |
| 12 | 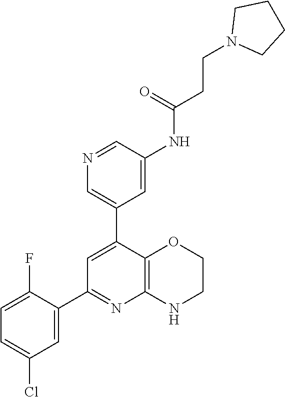 [SMILES: O=C(CCN1CCCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(pyrrolidin-1- yl)propanamide |
| 15 | 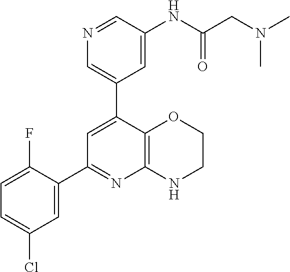 [SMILES: CN(C)CC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl }-2- (dimethylamino)acetamide |
| 16 | 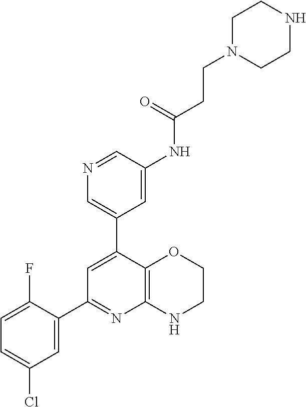 [SMILES: O=C(CCN1CCNCC1)Nc1cncc(-c2cc(-c3cc(Cl)ccc3F)nc3c2OCCN3)c1] | N-{5-[6-(5-chloro-2-fluorophenyl) 2H,3H,4H-pyrido[3,2-b][1,4]oxazin- 8-yl]pyridin-3-yl}-3-(piperazin-1- yl)propanamide |
| 17 | 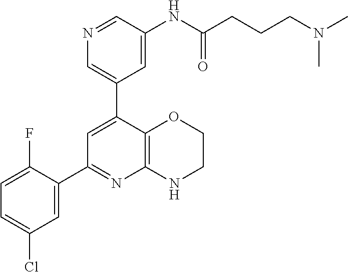 [SMILES: CN(C)CCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4- (dimethylamino)butanamide |
| 18 | 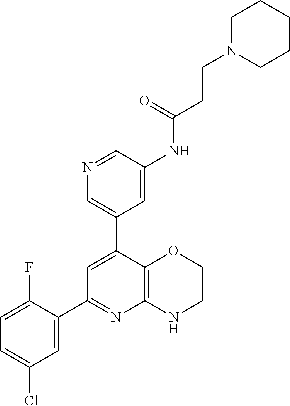 [SMILES: O=C(CCN1CCCCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(piperidin-1- yl)propanamide |
| 19 | 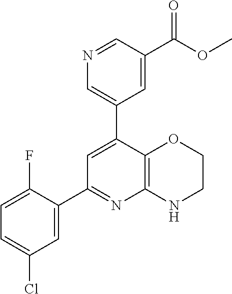 [SMILES: COC(=O)c1cncc(-c2cc(-c3cc(Cl)ccc3F)nc3c2OCCN3)c1] | methyl 5-[6-(5-chloro-2- fluorophenyl)-2H,3H,4H- pyrido[3,2-b][1,4]oxazin-8- yl]pyridine-3-carboxylate |
| 20 | 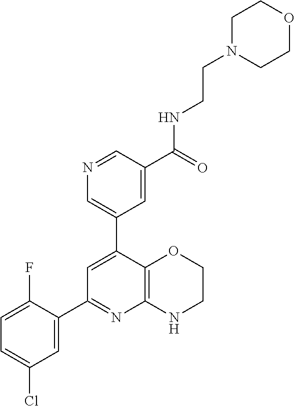 [SMILES: O=C(NCCN1CCOCC1)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[2-(piperazin-1- yl)ethyl]pyridine-3-carboxamide |
| 21 | 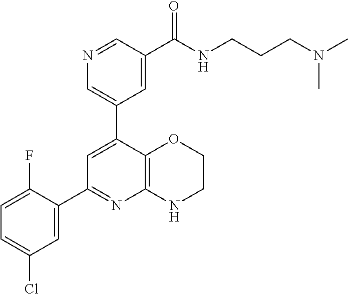 [SMILES: CN(C)CCCNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[3- (dimethylamino)propyl]pyridine-3- carboxamide |
| 22 | 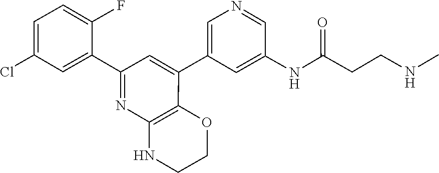 [SMILES: CNCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3- (methylamino)propanamide |
| 23 | 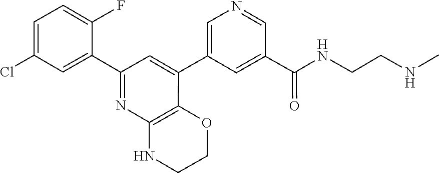 [SMILES: CNCCNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[2- (methylamino)ethyl]pyridine-3- carboxamide |
| 24 | 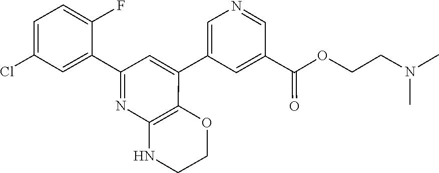 [SMILES: CN(C)CCOC(=O)c1cncc(-c2cc(-c3cc(Cl)ccc3F)nc3c2OCCN3)c1] | 2-(dimethylamino)ethyl 5-[6-(5- chloro-2-fluorophenyl)-2H,3H,4H- pyrido[3,2-b][1,4]oxazin-8- yl]pyridine-3-carboxylate |
| 25 | 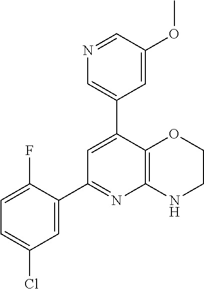 [SMILES: COc1cncc(-c2cc(-c3cc(Cl)ccc3F)nc3c2OCCN3)c1] | 3-[6-(5-chloro-2-fluorophenyl)- 2H,3H,4H-pyrido[3,2-b][1,4]oxazin- 8-yl]-5-methoxypyridine |
| 26 | 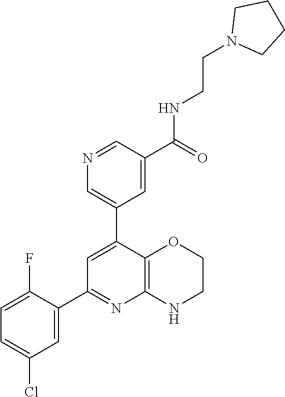 [SMILES: O=C(NCCN1CCCC1)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[2-(pyrrolidin-1- yl)ethyl]pyridine-3-carboxamide |
| 27 | 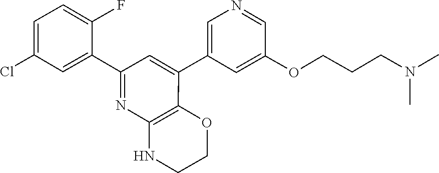 [SMILES: CN(C)CCCOc1cncc(-c2cc(-c3cc(Cl)ccc3F)nc3c2OCCN3)c1] | [3-({5-[6-(5-chloro-2-fluorophenyl)- 2H,3H,4H-pyrido[3,2-b][1,4]oxazin- 8-yl]pyridin-3- yl}oxy)propyl]dimethylamine |
| 28 | 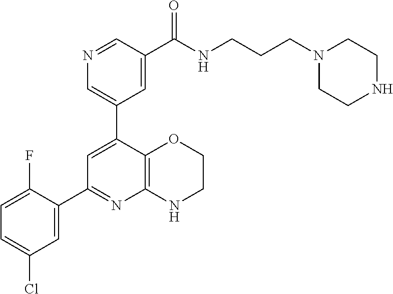 [SMILES: O=C(NCCCN1CCNCC1)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[3-(piperazin-1- yl)propyl]pyridine-3-carboxamide |
| 29 | 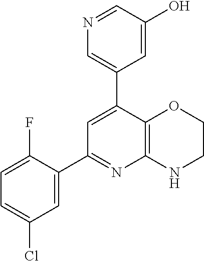 [SMILES: Oc1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]pyridin-3-ol |
| 30 | 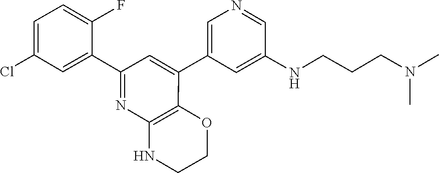 [SMILES: CN(C)CCCNc1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[3- (dimethylamino)propyl]pyridin-3- amine |
| 31 | 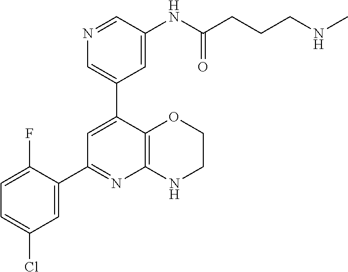 [SMILES: CNCCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4- (methylamino)butanamide |
| 32 | 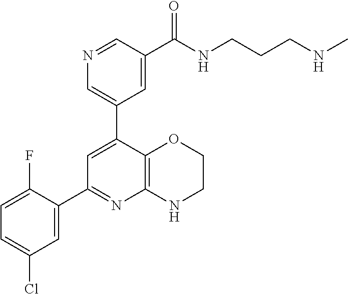 [SMILES: CNCCCNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[3- (methylamino)propyl]pyridine-3- carboxamide |
| 33 | 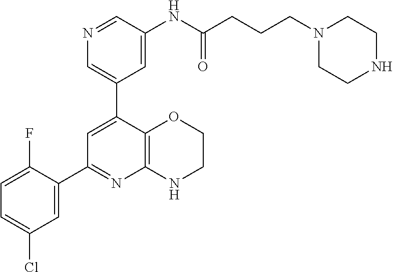 [SMILES: O=C(CCCN1CCNCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4-(piperazin-1- yl)butanamide |
| 34 | 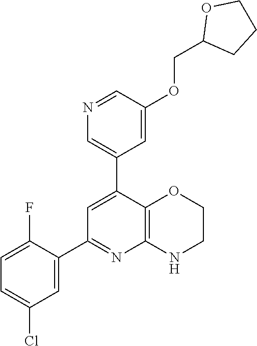 [SMILES: Fc1ccc(Cl)cc1-c1cc(-c2cncc(OCC3CCCO3)c2)c2c(n1)NCCO2] | 3-[6-(5-chloro-2-fluorophenyl)- 2H,3H,4H-pyrido[3,2-b][1,4]oxazin- 8-yl]-5-[(oxolan-2- yl)methoxy]pyridine |
| 36 | 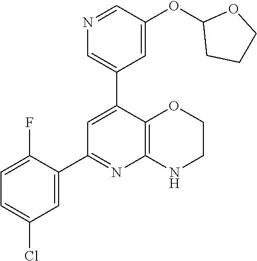 [SMILES: Fc1ccc(Cl)cc1-c1cc(-c2cncc(OC3CCCO3)c2)c2c(n1)NCCO2] | 3-[6-(5-chloro-2-fluorophenyl)- 2H,3H,4H-pyrido[3,2-b][1,4]oxazin- 8-yl]-5-(oxolan-3-yloxy)pyridine |
| 37 | 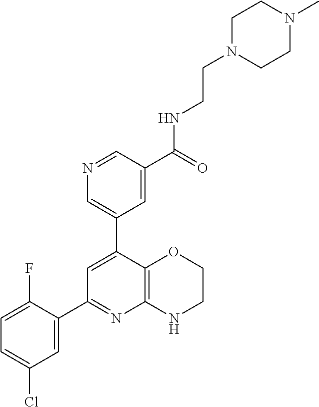 [SMILES: CN1CCN(CCNC(=O)c2cncc(-c3cc(-c4cc(Cl)ccc4F)nc4c3OCCN4)c2)CC1] | 5-[6-(5-chloro-2-fluorophenyl)- 2H,3H,4H-pyrido[3,2-b][1,4]oxazin- 8-yl]-N-[2-(4-methylpiperazin-1- yl)ethyl]pyridine-3-carboxamide |
| 38 | 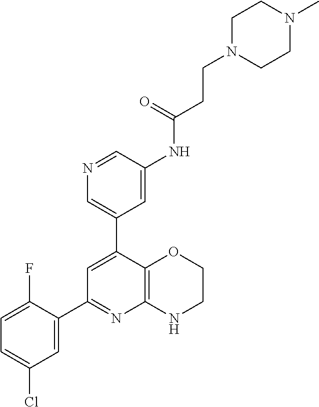 [SMILES: CN1CCN(CCC(=O)Nc2cncc(-c3cc(-c4cc(Cl)ccc4F)nc4c3OCCN4)c2)CC1] | N-{5-[6-(5-chloro-2-fluorophenyl)- 2H,3H,4H-pyrido[3,2-b][1,4]oxazin- 8-yl]pyridin-3-yl}-3-(4- methylpiperazin-1-yl)propanamide |
| 39 | 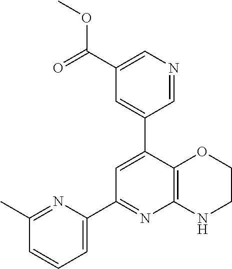 [SMILES: COC(=O)c1cncc(-c2cc(-c3cccc(C)n3)nc3c2OCCN3)c1] | methyl 5-[6-(6-methylpyridin-2-yl)- 2H,3H,4H-pyrido[3,2-b][1,4]oxazin- 8-yl]pyridine-3-carboxylate |
| 40 | 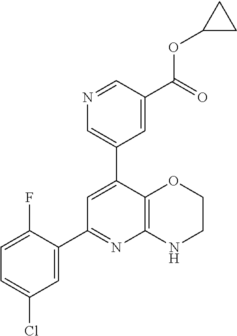 [SMILES: O=C(OC1CC1)c1cncc(-c2cc(-c3cc(Cl)ccc3F)nc3c2OCCN3)c1] | cyclopropyl 5-[6-(5-chloro-2- fluorophenyl)-2H,3H,4H- pyrido[3,2-b][1,4]oxazin-8- yl]pyridine-3-carboxylate |
| 41 | 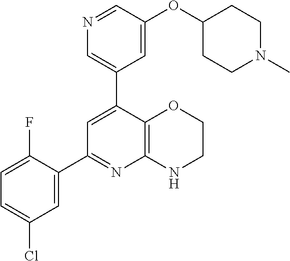 [SMILES: CN1CCC(Oc2cncc(-c3cc(-c4cc(Cl)ccc4F)nc4c3OCCN4)c2)CC1] | 3-[6-(5-chloro-2-fluorophenyl)- 2H,3H,4H-pyrido[3,2-b][1,4]oxazin- 8-yl]-5-[(1-methylpiperidin-4- yl)oxy]pyridine |
| 42 | 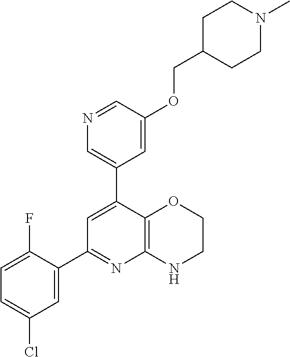 [SMILES: CN1CCC(COc2cncc(-c3cc(-c4cc(Cl)ccc4F)nc4c3OCCN4)c2)CC1] | 3-[6-(5-chloro-2-fluorophenyl)- 2H,3H,4H-pyrido[3,2-b][1,4]oxazin- 8-yl]-5-[(1-methylpiperidin-4- yl)methoxy]pyridine |
| 43 | 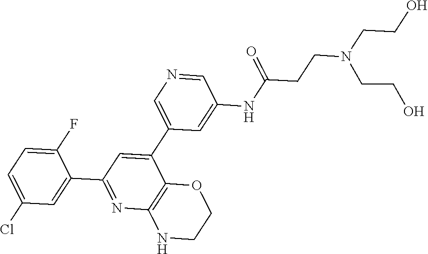 [SMILES: O=C(CCN(CCO)CCO)Nc1cncc(-c2cc(-c3cc(Cl)ccc3F)nc3c2OCCN3)c1] | 3-[bis(2-hydroxyethyl)amino]-N-{5- [6-(5-chloro-2-fluorophenyl)- 2H,3H,4H-pyrido[3,2-b][1,4]oxazin- 8-yl]pyridin-3-yl} propanamide |
| 44 | 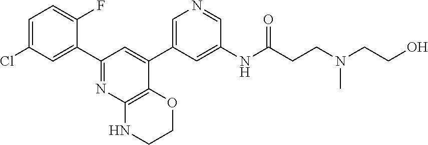 [SMILES: CN(CCO)CCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-[(2- hydroxyethyl)(methyl)amino] propanamide |
| 45 | 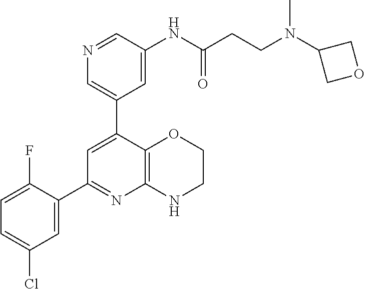 [SMILES: CN(CCC(=O)Nc1cncc(-c2cc(-c3cc(Cl)ccc3F)nc3c2OCCN3)c1)C1COC1] | N-{5-[6-(5-chloro-2-fluorophenyl)- 2H,3H,4H-pyrido[3,2-b][1,4]oxazin- 8-yl]pyridin-3-yl}-3- [methyl(oxetan-3- yl)amino]propanamide |
| 46 | 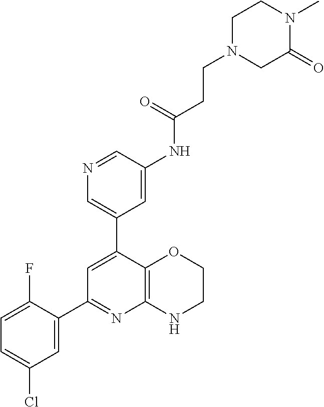 [SMILES: CN1CCN(CCC(=O)Nc2cncc(-c3cc(-c4cc(Cl)ccc4F)nc4c3OCCN4)c2)CC1=O] | N-{5-[6-(5-chloro-2-fluorophenyl)- 2H,3H,4H-pyrido[3,2-b][1,4]oxazin- 8-yl]pyridin-3-yl}-3-(4-methyl-3- oxopiperazin-1-yl)propanamide |
| 47 | 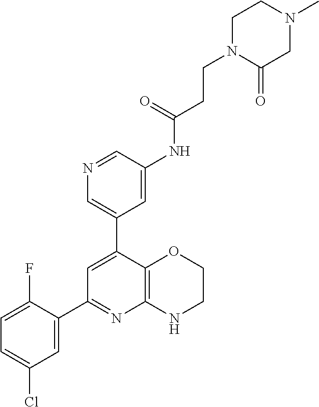 [SMILES: CN1CCN(CCC(=O)Nc2cncc(-c3cc(-c4cc(Cl)ccc4F)nc4c3OCCN4)c2)C(=O)C1] | N-{5-[6-(5-chloro-2-fluorophenyl)- 2H,3H,4H-pyrido[3,2-b][1,4]oxazin- 8-yl]pyridin-3-yl}-3-(4-methyl-2- oxopiperazin-1-yl)propanamide |
| 48 | 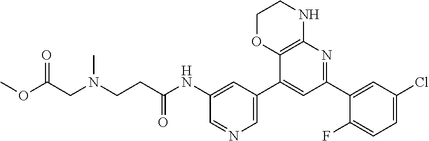 [SMILES: COC(=O)CN(C)CCC(=O)Nc1cncc(-c2cc(-c3cc(Cl)ccc3F)nc3c2OCCN3)c1] | methyl 2-{[2-({5-[6-(5-chloro-2- fluorophenyl)-2H,3H,4H- pyrido[3,2-b][1,4]oxazin-8- yl]pyridin-3- yl}carbamoyl)ethyl](methyl)amino} acetate |

According to a even more preferred embodiment, the present invention refers to a compound of formula (Ia), wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is —NR₃C(O)R₄;

R₃ is H or —(C₁-C₆)alkyl;

R₄ is selected from the group consisting of —(C₁-C₆)alkylene-NR_{A}R_{B} and —(C₁-C₆)alkylene-heterocycloalkyl;

R_{A} is H or is selected from the group consisting of —(C₁-C₆)alkyl and —(C₁-C₆)hydroxyalkyl;

R_{B} is —(C₁-C₆)alkyl;

and pharmaceutically acceptable salts thereof.

According to a preferred embodiment, the invention refers to at least one of the compounds of Formula (Ia) listed in the Table 2 below and pharmaceutical acceptable salts thereof. These compounds are particularly active on receptor ALK5, as shown in Table 12.

TABLE 2
List of preferred compounds of Formula (Ia)

| Example No. | Structure | Chemical Name |
|---|---|---|
| 8 | 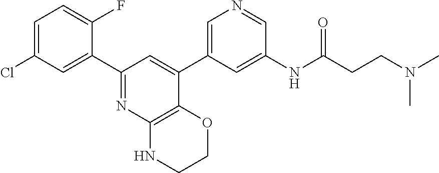 [SMILES: CN(C)CCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-2-yl}-3- (dimethylamino)propanamide |
| 9 | 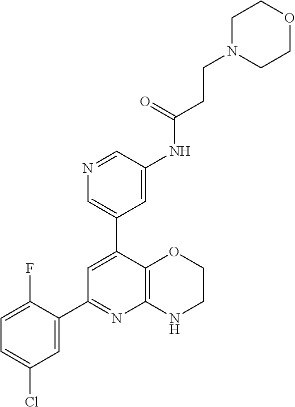 [SMILES: O=C(CCN1CCOCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(morpholin-4- yl)propanamide |
| 12 | 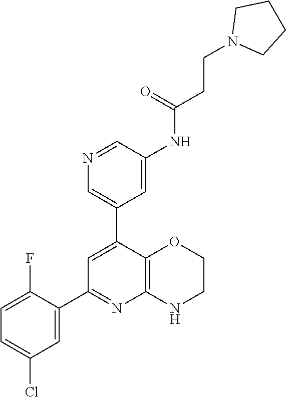 [SMILES: O=C(CCN1CCCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(pyrrolidin-1- yl)propanamide |
| 16 | 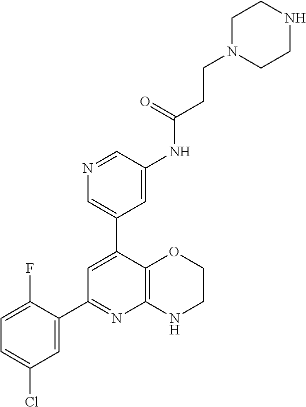 [SMILES: O=C(CCN1CCNCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(piperazin-1- yl)propanamide |
| 17 | 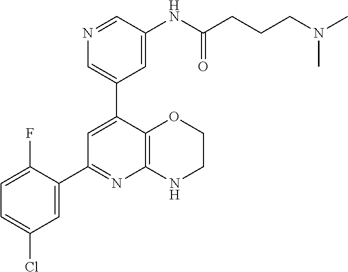 [SMILES: CN(C)CCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4- (dimethylamino)butanamide |
| 18 | 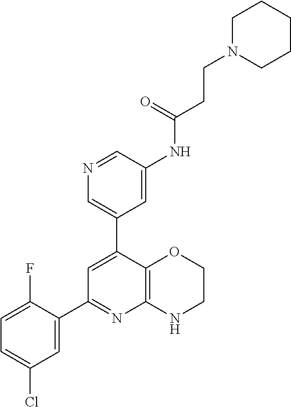 [SMILES: O=C(CCN1CCCCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(piperidin-1- yl)propanamide |
| 22 | 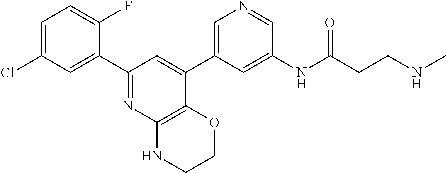 [SMILES: CNCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3- (methylamino)propanamide |
| 31 | 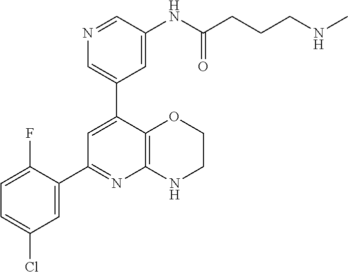 [SMILES: CNCCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4- (methylamino)butanamide |
| 33 | 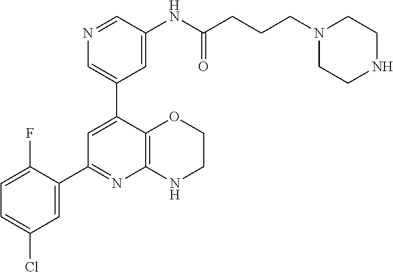 [SMILES: O=C(CCCN1CCNCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4-(piperazin-1- yl)butanamide |
| 38 | 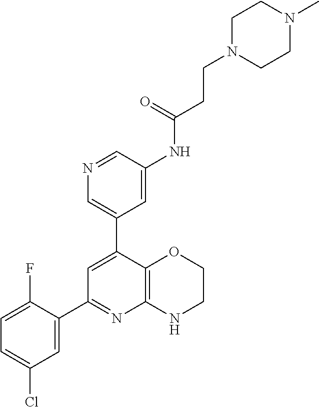 [SMILES: CN1CCN(CCC(=O)Nc2cncc(-c3cc(-c4cc(Cl)ccc4F)nc4c3OCCN4)c2)CC1] | N-{5-[6-(5-chloro-2-fluorophenyl)- 2H,3H,4H-pyrido[3,2-b][1,4]oxazin- 8-yl]pyridin-3-yl}-3-(4- methylpiperazin-1-yl)propanamide |
| 43 | 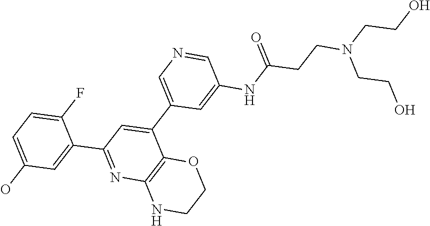 [SMILES: O=C(CCN(CCO)CCO)Nc1cncc(-c2cc(-c3cc(O)ccc3F)nc3c2OCCN3)c1] | 3-[bis(2-hydroxyethyl)amino]-N-{5- [6-(5-chloro-2-fluorophenyl)- 2H,3H,4H-pyrido[3,2-b][1,4]oxazin- 8-yl]pyridin-3-yl}propanamide |
| 44 | 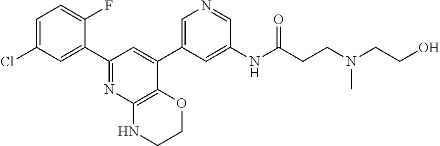 [SMILES: CN(CCO)CCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-[(2- hydroxyethyl)(methyl)amino] propanamide |
| 45 | 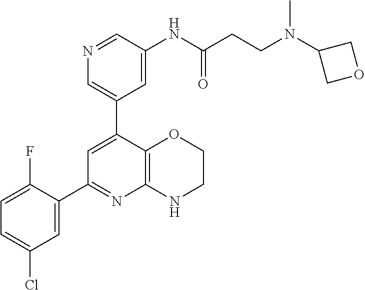 [SMILES: CN(CCC(=O)Nc1cncc(-c2cc(-c3cc(Cl)ccc3F)nc3c2OCCN3)c1)C1COC1] | N-{5-[6-(5-chloro-2-fluorophenyl)- 2H,3H,4H-pyrido[3,2-b][1,4]oxazin- 8-yl]pyridin-3-yl}-3- [methyl(oxetan-3- yl)amino]propanamide |

According to a even more preferred embodiment, the present invention refers to a compound of formula (Ia), wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is —NR₃C(O)R₄;

R₃ is H or —(C₁-C₆)alkyl;

R₄ is —(C₁-C₆)alkylene-NR_{A}R_{B};

R_{A} is H or is selected from the group consisting of —(C₁-C₆)alkyl and —(C₁-C₆)hydroxyalkyl;

R_{B} is —(C₁-C₆)alkyl;

and pharmaceutically acceptable salts thereof.

According to a preferred embodiment, the invention refers to at least one of the compounds of Formula (Ia) listed in the Table 3 below and pharmaceutical acceptable salts thereof. These compounds are particularly active on receptor ALK5, as shown in Table 12.

TABLE 3
List of preferred compounds of Formula (Ia)

| Example No. | Structure | Chemical Name |
|---|---|---|
| 8 | 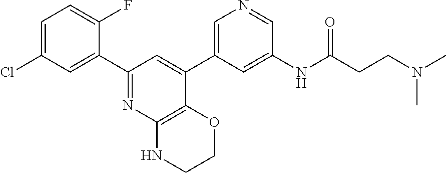 [SMILES: CN(C)CCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-2-yl}-3- (dimethylamino)propanamide |
| 17 | 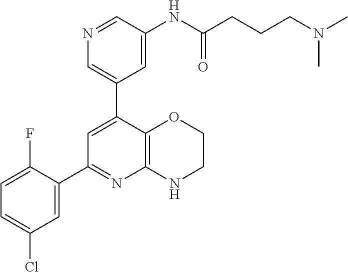 [SMILES: CN(C)CCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4- (dimethylamino)butanamide |
| 22 | 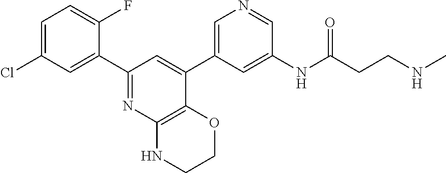 [SMILES: CNCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3- (methylamino)propanamide |
| 31 | 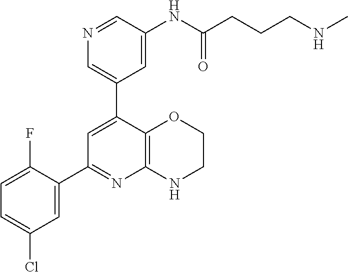 [SMILES: CNCCCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-4- (methylamino)butanamide |
| 44 | 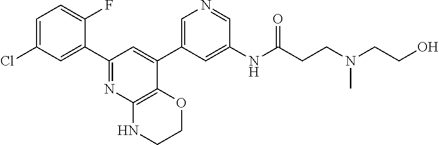 [SMILES: CN(CCO)CCC(=O)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-[(2- hydroxyethyl)(methyl)amino] propanamide |

According to another preferred embodiment, the present invention refers to a compound of formula (Ia), wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is —NR₃C(O)R₄;

R₃ is H or —(C₁-C₆)alkyl;

R₄ is —(C₁-C₆)alkylene-heterocycloalkyl, wherein in the heterocycloalkyl only one carbon atom is replaced by a heteroatom;

and pharmaceutically acceptable salts thereof.

According to a preferred embodiment, the invention refers to at least one of the compounds of Formula (Ia) listed in the Table 4 below and pharmaceutical acceptable salts thereof. These compounds are particularly active on receptor ALK5, as shown in Table 12.

TABLE 4
List of preferred compounds of Formula (Ia)

| Example No. | Structure | Chemical Name |
|---|---|---|
| 12 | 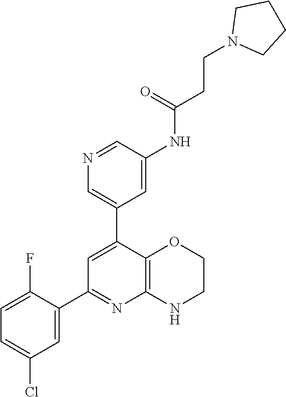 [SMILES: O=C(CCN1CCCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(pyrrolidin-1- yl)propanamide |
| 18 | 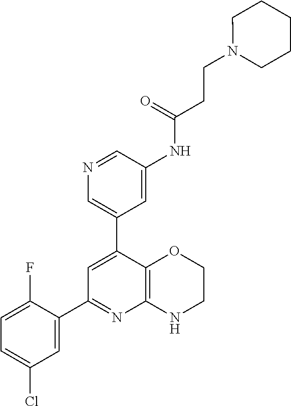 [SMILES: O=C(CCN1CCCCC1)Nc1cncc(-c2cc(-c3cc(Cl)ccc3F)nc3c2OCCN3)c1] | N-{5-[6-(5-chloro-2-fluorophenyl)- 2H,3H,4H-pyrido[3,2-b][1,4]oxazin- 8-yl]pyridin-3-yl}-3-(piperidin-1- yl)propanamide |

In another preferred embodiment, the present invention relates to compounds of formula (Ia), wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is —C(O)NR₃R₄;

R₃ is H or —(C₁-C₆)alkyl;

R₄ is selected from the group consisting of —(C₁-C₆)alkylene-NR_{A}R_{B} and —(C₁-C₆)alkylene-heterocycloalkyl, wherein said heterocycloalkyl is optionally substituted by one or more groups selected from oxo and —(C₁-C₆)alkyl;

R_{A} is H or is selected from the group consisting of —(C₁-C₆)alkyl and —(C₁-C₆)hydroxyalkyl;

R_{B} is selected from the group consisting of —(C₁-C₆)alkyl, heterocycloalkyl and —(C₁-C₆)hydroxyalkyl;

and pharmaceutically acceptable salts thereof.

According to a preferred embodiment, the invention refers to at least one of the compounds of Formula (Ia) listed in the Table 5 below and pharmaceutical acceptable salts thereof.

TABLE 5
List of preferred compounds of Formula (Ia)

| Example No. | Structure | Chemical Name |
|---|---|---|
| 20 | 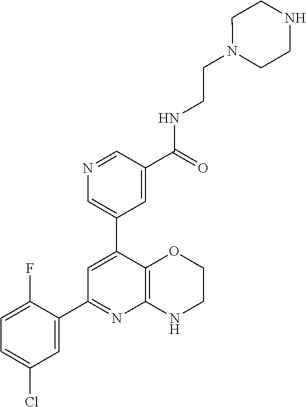 [SMILES: O=C(NCCN1CCNCC1)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[2-(piperazin-1- yl)ethyl]pyridine-3-carboxamide |
| 21 | 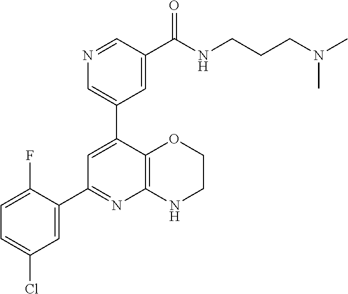 [SMILES: CN(C)CCCNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[3- (dimethylamino)propyl]pyridine-3- carboxamide |
| 23 | 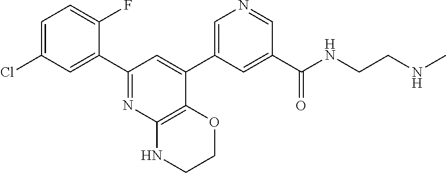 [SMILES: CNCCNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[2- (methylamino)ethyl]pyridine-3- carboxamide |
| 32 | 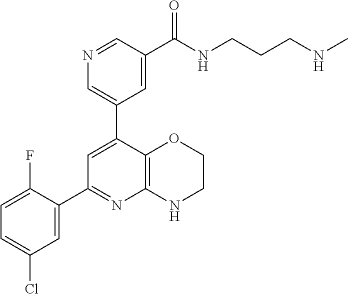 [SMILES: CNCCCNC(=O)c1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]-N-[3- (methylamino)propyl]pyridine-3- carboxamide |
| 37 | 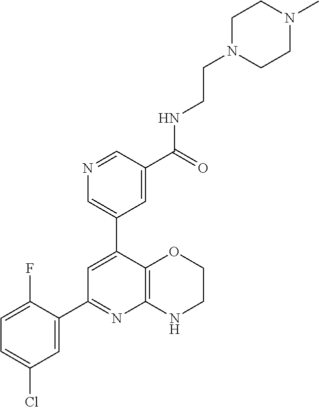 [SMILES: CN1CCN(CCNC(=O)c2cncc(-c3cc(-c4cc(Cl)ccc4F)nc4c3OCCN4)c2)CC1] | 5-[6-(5-chloro-2-fluorophenyl)- 2H,3H,4H-pyrido[3,2-b][1,4]oxazin- 8-yl]-N-[2-(4-methylpiperazin-1- yl)ethyl]pyridine-3-carboxamide |

In another preferred embodiment the present invention refers to a compound of formula (Ia), wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is —C(O)OR₅;

R₅ is selected from the group consisting of —(C₁-C₆)alkyl, —(C₁-C₆)alkylene-NR_{A}R_{B} and cycloalkyl;

R_{A} is H or is selected from the group consisting of —(C₁-C₆)alkyl and —(C₁-C₆)hydroxyalkyl;

R_{B} is selected from the group consisting of —(C₁-C₆)alkyl and —(C₁-C₆)hydroxyalkyl;

and pharmaceutically acceptable salts thereof.

According to a preferred embodiment, the invention refers to at least one of the compounds of Formula (Ia) listed in the Table 6 below and pharmaceutically acceptable salts thereof.

TABLE 6
List of preferred compounds of Formula (Ia)

| Example No. | Structure | Chemical Name |
|---|---|---|
| 19 | 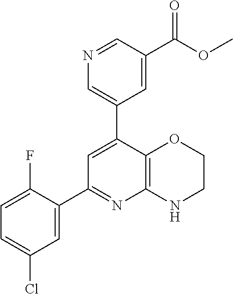 [SMILES: COC(=O)c1cncc(-c2cc(-c3cc(Cl)ccc3F)nc3c2OCCN3)c1] | methyl 5-[6-(5-chloro-2- fluorophenyl)-2H,3H,4H- pyrido[3,2-b][1,4]oxazin-8- yl]pyridine-3-carboxylate |
| 24 | 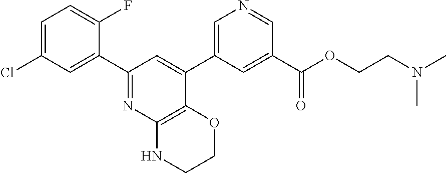 [SMILES: CN(C)CCOC(=O)c1cncc(-c2cc(-c3cc(Cl)ccc3F)nc3c2OCCN3)c1] | 2-(dimethylamino)ethyl 5-[6-(5- chloro-2-fluorophenyl)-2H,3H,4H- pyrido[3,2-b][1,4]oxazin-8- yl]pyridine-3-carboxylate |
| 40 | 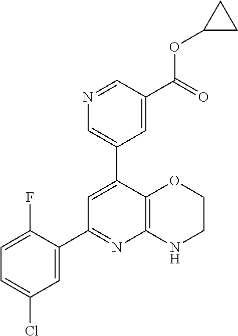 [SMILES: O=C(OC1CC1)c1cncc(-c2cc(-c3cc(Cl)ccc3F)nc3c2OCCN3)c1] | cyclopropyl 5-[6-(5-chloro-2- fluorophenyl)-2H,3H,4H- pyrido[3,2-b][1,4]oxazin-8- yl]pyridine-3-carboxylate |

According to another preferred embodiment, the present invention refers to a compound of formula (Ia), wherein

R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;

R₂ is —OR₆;

R₆ is H or is selected from the group consisting of —(C₁-C₆)alkyl, heterocycloalkyl, —(C₁-C₆)alkylene-NR_{A}R_{B}, —(C₁-C₆)alkylene-heterocycloalkyl, wherein said heterocycloalkyl is optionally substituted by one or more —(C₁-C₆)alkyl;

R_{A} is H or is selected from the group consisting of —(C₁-C₆)alkyl and —(C₁-C₆)hydroxyalkyl;

R_{B} is selected from the group consisting of —(C₁-C₆)alkyl and —(C₁-C₆)hydroxyalkyl;

and pharmaceutically acceptable salts thereof.

According to a preferred embodiment, the invention refers to at least one of the compounds of Formula (Ia) listed in the Table 7 below and pharmaceutical acceptable salts thereof.

TABLE 7
List of preferred compounds of Formula (Ia)

| Example No. | Structure | Chemical Name |
|---|---|---|
| 25 | 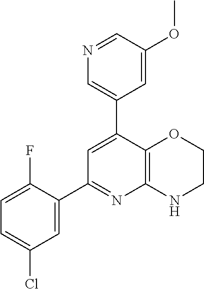 [SMILES: COc1cncc(-c2cc(-c3cc(Cl)ccc3F)nc3c2OCCN3)c1] | 3-[6-(5-chloro-2-fluorophenyl)- 2H,3H,4H-pyrido[3,2-b][1,4]oxazin- 8-yl]-5-methoxypyridine |
| 27 | 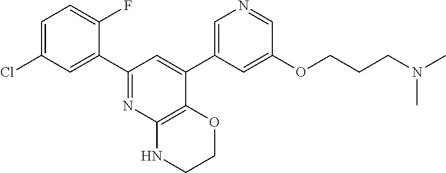 [SMILES: CN(C)CCCOc1cncc(-c2cc(-c3cc(Cl)ccc3F)nc3c2OCCN3)c1] | [3-({5-[6-(5-chloro-2-fluorophenyl)- 2H,3H,4H-pyrido[3,2-b][1,4]oxazin- 8-yl]pyridin-3- yl}oxy)propyl]dimethylamine |
| 29 | 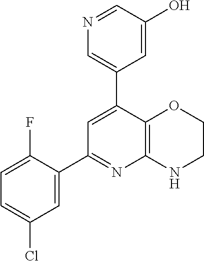 [SMILES: Oc1cncc(-c2cc(-c3cc(Cl)ccc3F)nc3c2OCCN3)c1] | 5-[6-(5-chloro-2-fluorophenyl)- 2H,3H,4H-pyrido[3,2-b][1,4]oxazin- 8-yl]pyridin-3-ol |
| 36 | 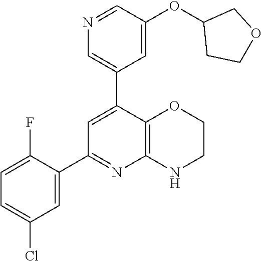 [SMILES: Fc1ccc(Cl)cc1-c1cc(-c2cncc(OC3CCOC3)c2)c2c(n1)NCCO2] | 3-[6-(5-chloro-2-fluorophenyl)- 2H,3H,4H-pyrido[3,2-b][1,4]oxazin- 8-yl]-5-(oxolan-3-yloxy)pyridine |
| 41 | 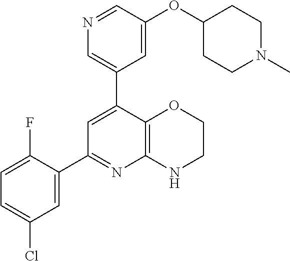 [SMILES: CN1CCC(Oc2cncc(-c3cc(-c4cc(Cl)ccc4F)nc4c3OCCN4)c2)CC1] | 3-[6-(5-chloro-2-fluorophenyl)- 2H,3H,4H-pyrido[3,2-b][1,4]oxazin- 8-yl]-5-[(1-methylpiperidin-4- yl)oxy]pyridine |
| 42 | 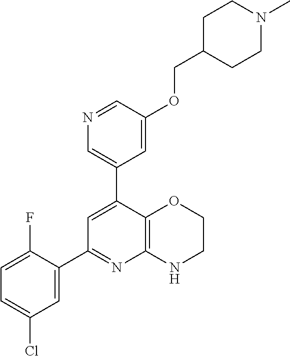 [SMILES: CN1CCC(COc2cncc(-c3cc(-c4cc(Cl)ccc4F)nc4c3OCCN4)c2)CC1] | 3-[6-(5-chloro-2-fluorophenyl)- 2H,3H,4H-pyrido[3,2-b][1,4]oxazin- 8-yl]-5-[(1-methylpiperidin-4- yl)methoxy]pyridine |

In a particularly preferred embodiment the present invention refers to a compound of formula (I), wherein A is group A2

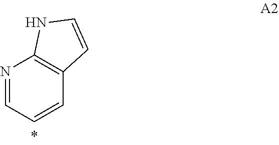

represented by the formula (Ib)

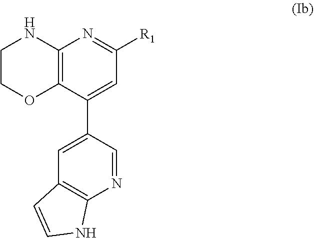

wherein R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms; and pharmaceutically acceptable salts thereof.

According to a preferred embodiment, the invention refers to the compound of Formula (Ib) listed in the Table 8 below and pharmaceutical acceptable salts thereof.

TABLE 8
Preferred compounds of Formula (Ib)

| Example No. | Structure | Chemical Name |
|---|---|---|
| 13 | 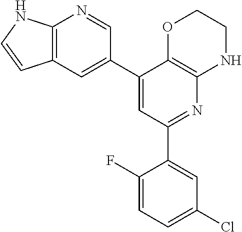 [SMILES: Fc1ccc(Cl)cc1-c1cc(-c2cnc3[nH]ccc3c2)c2c(n1)NCCO2] | 6-(5-chloro-2-fluorophenyl)-8-{1H- pyrrolo[2,3-b]pyridin-5-yl}- 2H,3H,4H-pyrido[3,2-b] [1,4]oxazine |

In a equally preferred embodiment the present invention refers to a compound of formula (I), wherein A is group A3

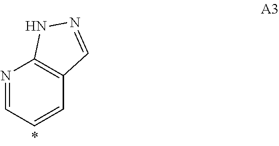

represented by the formula (Ic)

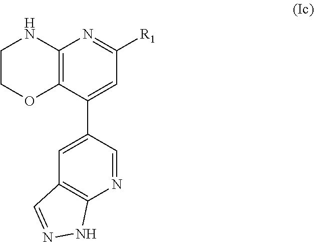

wherein R1 is aryl optionally substituted by one or more groups selected from halogen atoms and —(C₁-C₆)alkyl; or R1 is heteroaryl containing one or more heteroatoms selected from N, O and S, wherein said heteroaryl is optionally substituted by one or more groups selected from —(C₁-C₆)alkyl and halogen atoms; and pharmaceutically acceptable salts thereof.

According to a even more preferred embodiment, the invention refers to at least one of the compounds represented by Formula (Ic) listed in the Table 9 below and pharmaceutical acceptable salts thereof.

TABLE 9
List of preferred compounds of Formula (Ic)

| Example No. | Structure | Chemical Name |
|---|---|---|
| 10 | 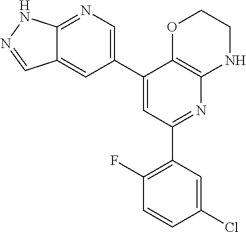 [SMILES: Fc1ccc(Cl)cc1-c1cc(-c2cnc3[nH]ncc3c2)c2c(n1)NCCO2] | 6-(5-chloro-2-fluorophenyl)-8-{1H- pyrazolo[3,4-b]pyridin-5-yl}- 2H,3H,4H-pyrido[3,2-b] [1,4]oxazine |
| 35 | 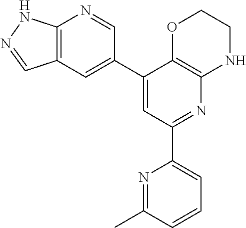 [SMILES: Cc1cccc(-c2cc(-c3cnc4[nH]ncc4c3)c3c(n2)NCCO3)n1] | 2-methyl-6-(8-{1H-pyrazolo[3,4-b] pyridin-5-yl}-2H,3H,4H- pyrido[3,2-b][1,4]oxazin-6- yl)pyridine |

In a equally preferred embodiment the present invention refers to a compound of formula (I), wherein A is group A₄

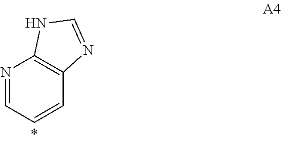

represented by the formula (Id)

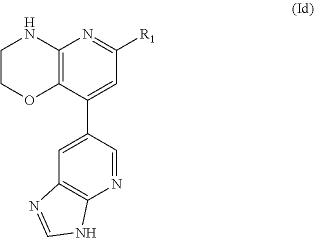

wherein R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms; and pharmaceutically acceptable salts thereof.

According to a even more preferred embodiment, the invention refers to the compound represented by Formula (Id) listed in the Table 10 below and pharmaceutical acceptable salts thereof.

TABLE 10
Preferred compound of Formula (Id)

| Example No. | Structure | Chemical Name |
|---|---|---|
| 14 | 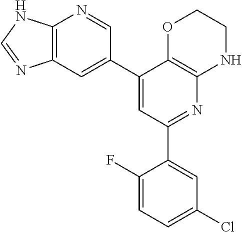 [SMILES: Fc1ccc(Cl)cc1-c1cc(-c2cnc3[nH]cnc3c2)c2c(n1)NCCO2] | 6-(5-chloro-2-fluorophenyl)-8-{3H- imidazo[4,5-b]pyridin-6-yl}- 2H,3H,4H-pyrido[3,2-b] [1,4]oxazine |

In a equally preferred embodiment the present invention refers to a compound of formula (I), wherein A is group A5

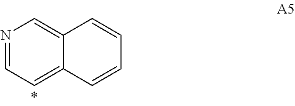

represented by the formula (Ie)

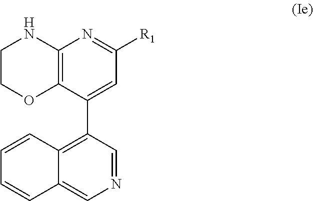

wherein R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms; and pharmaceutically acceptable salts thereof.

According to a even more preferred embodiment, the invention refers to the compound represented by Formula (Ie) listed in the Table 11 below and pharmaceutical acceptable salts thereof.

TABLE 11
List of preferred compounds of Formula (Ie)

| Example No. | Structure | Chemical Name |
|---|---|---|
| 2 | 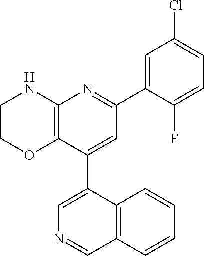 [SMILES: Fc1ccc(Cl)cc1-c1cc(-c2cncc3ccccc23)c2c(n1)NCCO2] | 4-[6-(5-chloro-2-fluorophenyl)- 2H,3H,4H-pyrido[3,2-b][1,4]oxazin- 8-yl]isoquinoline |
| 3 | 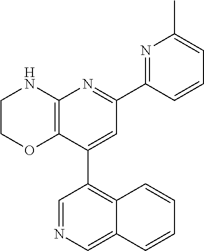 [SMILES: Cc1cccc(-c2cc(-c3cncc4ccccc34)c3c(n2)NCCO3)n1] | 4-[6-(6-methylpyridin-2-yl)- 2H,3H,4H-pyrido[3,2-b][1,4]oxazin- 8-yl]isoquinoline |
| 4 | 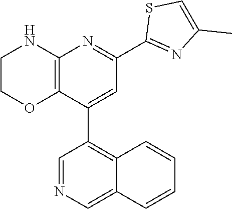 [SMILES: Cc1csc(-c2cc(-c3cncc4ccccc34)c3c(n2)NCCO3)n1] | 4-[6-(4-methyl-1,3-thiazol-2-yl)- 2H,3H,4H-pyrido[3,2-b][1,4]oxazin- 8-yl]isoquinoline |

In another preferred embodiment, R1 is selected from the group consisting of phenyl, optionally substituted by one or more groups selected from fluorine and chlorine; pyridinyl, optionally substituted by one or more methyl; and thiazolyl, optionally substituted by one or more methyl.

The compounds of formula (I) including all the compounds or at least one of the here above listed can be generally prepared according to the procedure outlined in detail in the Schemes shown below using generally known methods.

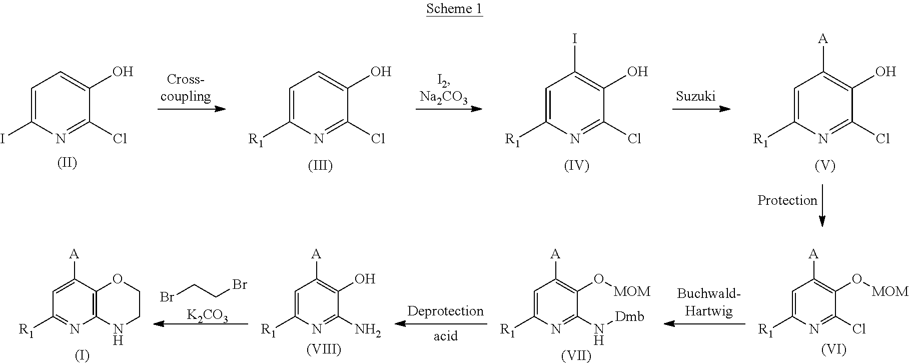

In one embodiment of the present invention, compounds of formula (I) may be prepared as described in Scheme 1, starting from commercially available compound (II).

Compounds (III), wherein R₁ is as defined above, may be prepared from compound (II) by Suzuky coupling using Pd(PPh₃)₄ as catalyst in a mixture of solvents like, for example, toluene and water.

Compounds (IV) may be prepared from compounds (III) by iodination using, for example, I₂ and Na₂CO₃.

Compounds (V) may be prepared from compounds (IV) by cross coupling reaction with aryl or heteroaryl boronate esters or boronic acids derivatives and palladium catalyst.

Compounds (VI) may be prepared from compounds (V) by protecting the OH with a protecting group like, for example, methoxy methyl chloride.

Compounds (VII) may be prepared from compounds (VI) by N-arylation with dimethoxy benzyl amine under standard Buchwald-Hartwig amination conditions.

Compounds (VIII) may be prepared from compounds (VII) by removal of protecting groups under acidic conditions, using, for example, trifluoro acetic acid in a solvent like, for example, dichloromethane at room temperature.

Compounds of formula (I) may be prepared by cyclization with an alkylating agent like, for example, 1,2-dibromoethane.

In another embodiment of the present invention, compounds of formula (I) can be prepared as described in Scheme 2:

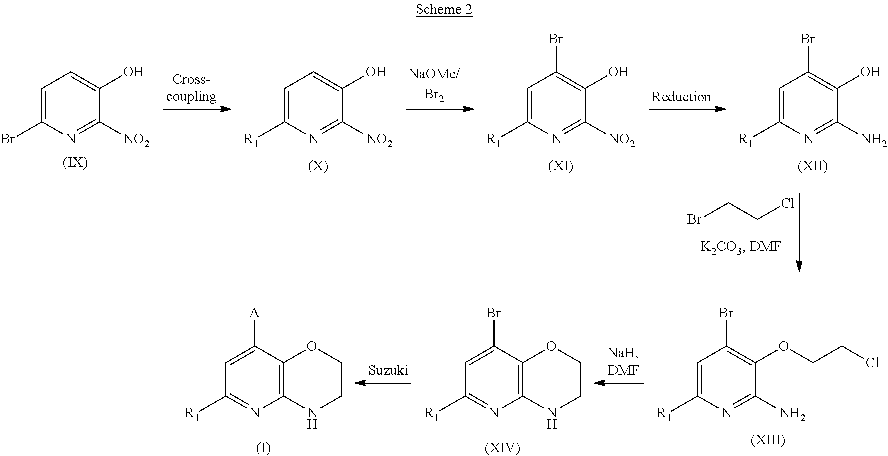

Compounds (X) may be prepared starting from commercially available compound (IX) by Suzuky coupling using Pd(PPh₃)₄ as catalyst in a mixture of solvents like, for example, toluene and water.

Compounds (XI) may be prepared by bromuration of compounds of formula (X) using for example bromine in the presence of sodium methoxide in a solvent like, for example, dry MeOH at low temperature like, for example, 0° C.

Compounds (XII) may be prepared from compounds (XI) by reduction of the nitro group using for example sodium borohydride and nickel(II) chloride hexahydrate in MeOH and dry THF as solvents.

Compounds (XIII) may be prepared from compounds (XII) by alkylation with 1-bromo-2-chloroethane in the presence of a base like, for example, potassium carbonate in dry DMF.

Compounds of formula (XIV) may be prepared from compounds (XIII) by base-catalysed intramolecular cyclization, using, for example, NaH in dry DMF at room temperature.

Compounds of formula (I) may be prepared from compounds of formula (XIV) by cross coupling standard procedures, using aryl or heteroaryl boronate esters or boronic acids derivatives.

Alternatively, compounds of formula (I) may be prepared as described in Scheme 3 starting from commercially available 4,6-dibromo-2-nitropyridin-3-ol (Compound XV).

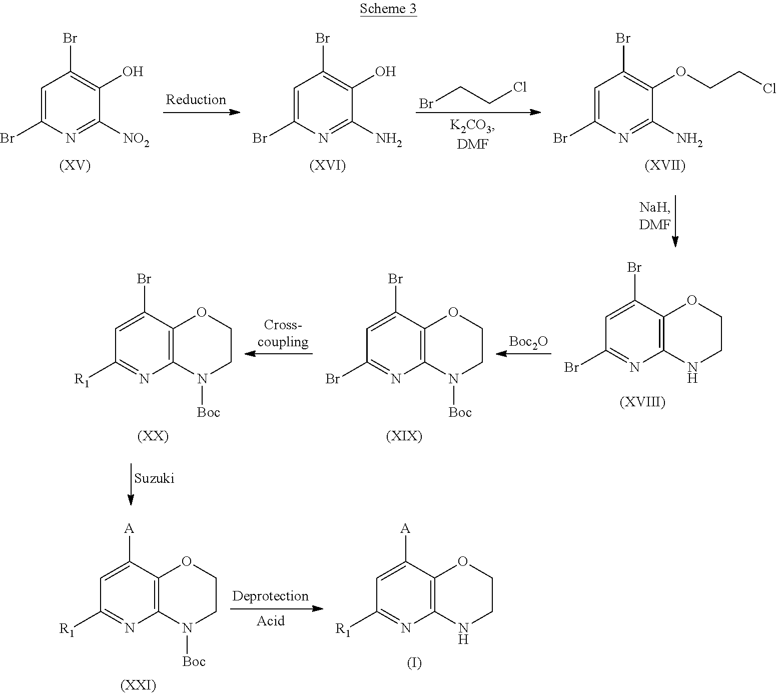

Compound (XVI) may be prepared from compound (XV) by reduction of nitro group using sodium borohydride and nickel(II) chloride hexahydrate in a mixture of dry MeOH and dry THF.

Compound (XVII) may be prepared from compound (XVI) by alkylation with 1-bromo-2-chloroethane in the presence of a base like, for example, potassium carbonate in dry DM.

Compound of formula (XVIII) may be prepared from compound (XVII) by base-catalyzed intramolecular cyclization using a base like, for example, NaH in dry DMF. Compound of formula (XIX) may be prepared from compound (XVIII) by protection of the nitrogen using di-tert-butyl dicarbonate (Boc anhydride, Boc₂O) in the presence of a base like, for example, lithium bis(trimethylsilyl)amide in a solvent like, for example, THF.

Compounds of formula (XX) may be prepared from compound (XIX) by introduction of R₁ via Suzuky or Stille coupling.

Compounds of formula (XXI) may be prepared from compounds (XX) by Suzuky coupling with aryl or heteroaryl boronate esters or boronic acids derivatives.

Compound of formula (I) may be prepared from compounds (XXI) by deprotection using, for example, trifluoroacetic acid in a solvent like, for example, DCM.

In a further aspect the present invention relates to the use of compounds of formula (IV), (V), (VIII), (X), (XI), (XII), (XIII), (XIV), (XX) and (XXI) as intermediates in the preparation of compounds of formula (I) as above described.

The compounds of formula (I) of the present invention have surprisingly been found to effectively inhibit the receptor ALK5. Advantageously, the inhibition of ALK5 may result in efficacious treatment of the diseases or condition wherein the ALK5 signaling is involved.

In this respect, it has now been found that the compounds of formula (I) of the present invention have an inhibitory drug potency expressed as half maximal inhibitory concentration (IC50) on ALK5 lower or equal than 10 nM as shown in the present experimental part.

Preferably, the compounds of the present invention have an IC50 on ALK5 between 5 and 10 nM. Even more preferably, the compounds of the present invention have an IC50 on ALK5 lower than 1 nM.

The present invention also provides a pharmaceutical composition comprising a compound of formula (I) or a pharmaceutically acceptable salt thereof in admixture with one or more pharmaceutically acceptable carrier or excipient, either alone or in combination with one or more further active ingredient.

In one aspect, the present invention refers to a compound of formula (I) or a pharmaceutically acceptable salt thereof, for use as a medicament.

In a further aspect, the invention refers to the use of a compound of formula (I) of the invention, or a pharmaceutically acceptable salt thereof, in the manufacture of a medicament for the prevention and/or treatment of a disease, disorder or condition associated with dysregulated ALK5 signaling pathway.

In a preferred embodiment, the invention refers to a compound of formula (I) or a pharmaceutically acceptable salt thereof, for use in the prevention and/or treatment of a disease, disorder or condition associated with dysregulated ALK5 signaling pathway. In one embodiment, the present invention refers to a compound of formula (I) useful for the prevention and/or treatment of fibrosis and/or diseases, disorders, or conditions that involve fibrosis.

The invention also provides a method for the prevention and/or treatment of a disease, disorder or condition associated with dysregulated ALK5 signaling pathway, said method comprising administering to a patient in need of such treatment a therapeutically effective amount of a compound of the invention.

In particular the invention refers to a method for the prevention and/or treatment of fibrosis and/or diseases, disorders, or conditions that involve fibrosis, wherein said method comprises the administration of a proper amount of a compound of formula (I) to a patient in the need thereof.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract. Preferably, the compounds of formula (I) of the present invention, or a pharmaceutical composition comprising a compound of formula (I), are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) of the present invention, or a pharmaceutical composition comprising a compound of formula (I), are useful for the treatment of idiopathic pulmonary fibrosis (IPF).

The methods of treatment of the invention comprise administering a safe and effective amount of a compound of formula (I) or a pharmaceutically acceptable salt thereof to a patient in need thereof. As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan. The compounds of formula (I) or pharmaceutically acceptable salts thereof may be administered once or according to a dosing regimen wherein a number of doses are administered at varying intervals of time for a given period of time. Typical daily dosages may vary depending upon the particular route of administration chosen.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation.

Preferably, the compounds of the present invention are administered orally or by inhalation. More preferably, the compounds of the present invention are administered by inhalation.

In one preferred embodiment, the pharmaceutical composition comprising the compound of formula (I) is a solid oral dosage form such as tablets, gelcaps, capsules, caplets, granules, lozenges and bulk powders. The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In one embodiment, the pharmaceutical composition comprising the compound of formula (I) is a tablet.

In a further embodiment, the pharmaceutical composition comprising a compound of formula (I) is a liquid oral dosage forms such as aqueous and non-aqueous solutions, emulsions, suspensions, syrups, and elixirs. Such liquid dosage forms can also contain suitable known inert diluents such as water and suitable known excipients such as preservatives, wetting agents, sweeteners, flavorants, as well as agents for emulsifying and/or suspending the compounds of the invention. The compounds of the invention may be injected, for example, intravenously, in the form of an isotonic sterile solution.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention are administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of formula (I) according to the invention, in form of a single- or multi-dose dry powder inhaler or a metered dose inhaler.

All preferred groups or embodiments described above for compounds of formula (I) may be combined among each other and apply as well mutatis mutandis.

The various aspects of the invention described in this application are illustrated by the following examples which are not meant to limit the invention in any way.

### Preparations of Intermediates and Examples

Chemical Names of the compounds were generated with Structure To Name Enterprise 10.0 Cambridge Software.

All reagents, for which the synthesis is not described in the experimental part, are either commercially available, or are known compounds or may be formed from known compounds by known methods by a person skilled in the art.

In the procedures that follow, some of the starting materials are identified through an “Intermediate” or “Example” number with indications on step number. This is provided merely for assistance to the skilled chemist.

A “similar” or “analogous” procedure means that such a procedure may involve minor variations, for example reaction temperature, reagent/solvent amount, reaction time, work-up conditions or chromatographic purification conditions.

### Abbreviation—Meaning

BINAP=(±)-2,2′-Bis(diphenylphosphino)-1,1′-binaphthalene
Boc=Butyloxy carbonyl
Boc2O=Di-tert butyl dicarbonate
c-Hex=Cyclohexane
DCM=Dichloromethane
DIPEA=N,N-Diisopropylethylamine
DMB=2,4-dimethoxybenzyl
DMF=Dimethylformamide
DMSO=Dimethylsulfoxide
DTBPF=1,1′-Bis(di-tert-butylphosphino)ferrocene
EtOAc=Ethyl acetate
Et₂O=Diethyl ether
FC=flash chromatography
h=hour
hrs=hours
HATU=2-(7-Aza-1H-benzotriazole-1-yl)-1,1,3,3-tetramethyluronium
hexafluorophosphate
HCl=Hydrochloric acid
HCOOH=Formic acid
K₂CO₃=Potassium carbonate
KOAc=Potassium acetate
LC-MS=liquid chromatography/mass spectrometry
MeCN=Acetonitrile
MeOH=Methanol
MOM=Methoxy methyl
Na₂CO₃=Sodium carbonate
NaOMe=Sodium methoxide
NH₃=Ammonia
NaHCO₃=Sodium bicarbonate
NaOH═Sodium hydroxide
Na₂S₂O₃=Sodium thiosulfate
PdCl₂(dtbpf)=[1,1′-Bis(di-tert-butylphosphino)ferrocene]dichloropalladium(II)
Pd(dba)₂=Bis(dibenzylideneacetone)palladium(0)
Pd₂(dba)₃=Tris(dibenzylideneacetone)dipalladium(0)
Pd(dppf)Cl₂ DCM=[1,1′-Bis(diphenylphosphino)ferrocene]dichloropalladium(II), complex with dichloromethane
Pd(PPh₃)₄=Tetrakis(triphenylphosphine)palladium(0)
PLS=Parallel Liquid Synthesizer
RT=room temperature
Sat.=saturated
SCX=Strong Cation Exchange
tBu=tert-butyl
TEA=Triethylamine
TFA=Trifluoroacetic acid
THF=Tetrahydrofuran

### General Experimental Details and Methods

Analytical Method

Instruments, Materials and Methods Employed for Analyses

¹H-NMR spectra were performed on a Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with trans mitter offset frequency shift, or on AgilentVNMRS-500 or on a Bruker Avance 400 spectrometers. Chemical shift are reported as 6 values in ppm relative to trimethylsilane (TMS) as an internal standard. Coupling constants (J values) are given in hertz (Hz) and multiplicities are reported using the following abbreviation (br=broad signal, s=singlet, d=doublet, dd=doublet of doublets, ddd=doublet of doublet of doublets, t=triplet, dt=doublet of triplets, q=quartet, m=multiplet).

LC/UV/MS Analytical Methods

LC/MS retention times are estimated to be affected by an experimental error of +0.5 min. LCMS may be recorded under the following conditions: diode array DAD chromatographic traces, mass chromatograms and mass spectra may be taken on UPLC/PDA/MS Acquity™ system coupled with Micromass ZQ™ or Waters SQD single quadrupole mass spectrometer operated in positive and/or negative electron spray ES ionization mode and/or Fractionlynx system used in analytical mode coupled with ZQ™ single quadrupole operated in positive and/or negative ES ionisation mode. Quality Control methods used operated under low pH conditions or under high pH conditions:

Method 1, low pH conditions column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 1 mL/min.

The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 2, high pH conditions: column: Acquity Kinetex 1.7 um EVO C18 100A, 2.1×50 mm, the column temperature was 40° C.; mobile phase solvent A was 10 mM aqueous solution of NH4HCO3 adjusted to pH=10 with ammonia, mobile phase solvent B MeCN. The flow rate was 1 mL/min. The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

### Preparations of Intermediates

### Intermediate 1: 2-chloro-6-(5-chloro-2-fluorophenyl)pyridin-3-ol

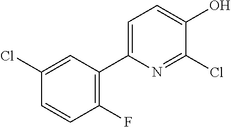 [SMILES: Oc1ccc(-c2cc(Cl)ccc2F)nc1Cl]

In a suitable vial, charged with 2-chloro-6-iodopyridin-3-ol (500 mg, 1.96 mmol), 5-chloro-2-fluorophenylboronic acid (341 mg, 1.96 mmol), Pd(PPh₃)₄ (113 mg, 0.10 mmol) and NaHCO₃ (333 mg, 3.91 mmol), a mixture of toluene, ethanol and water (16 mL, 6:1:1 ratio) was added. The vessel was sealed, evacuated and backfilled with N₂ (3×), then heated at 90° C. for 6 hrs. The mixture was cooled down and filtered through a Celite® pad, washing with EtOAc. The filtrate was washed with acidified brine (2×), then filtered through a phase separator and evaporated under vacuum. The crude material was purified by FC on Biotage silica gel (c-Hex 100%, then DCM 100%), to provide the title compound (367 mg, 1.42 mmol, 73% yield) as pale orange solid.

LC-MS (ESI): m/z (M+1): 258.2 (Method 1)

### Intermediate 2: 2-chloro-6-(5-chloro-2-fluorophenyl)-4-iodopyridin-3-ol

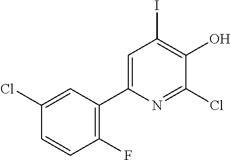 [SMILES: Oc1c(I)cc(-c2cc(Cl)ccc2F)nc1Cl]

To a solution of Intermediate 1 (1.47 g, 5.68 mmol) and Na₂CO₃ (1.32 g, 12.5 mmol) in water (14.5 mL), iodine (1.44 g, 5.68 mmol) was added. The mixture was stirred at RT for 1 h. Residual iodine was quenched with Na₂S₂O₃ 10% aqueous solution, then the mixture was acidified with 2 N HCl solution. The resulting precipitate was collected by filtration and washed with cold water, affording the title compound (2.13 g, 5.55 mmol, 98% yield) as an ivory solid.

LC-MS (ESI): m/z (M+1): 383.9 (Method 1)

### Intermediate 3: 2′-chloro-6′-(5-chloro-2-fluorophenyl)-[3,4′-bipyridin]-3′-ol

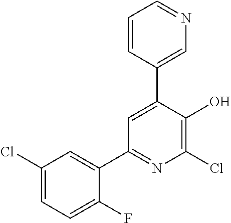 [SMILES: Oc1c(-c2cccnc2)cc(-c2cc(Cl)ccc2F)nc1Cl]

In a suitable vial charged with Intermediate 2 (1.10 g, 2.78 mmol), 3-pyridinylboronic acid (0.41 g, 3.33 mmol), Pd(PPh₃)₄ (0.16 g, 0.14 mmol) and NaHCO₃ (0.47 g, 5.56 mmol), a mixture of toluene, ethanol and water (10 mL, 6:1:1 ratio) was added. The vessel was sealed, evacuated and backfilled with N₂ (3×), then heated at 90° C. overnight. The mixture was cooled down to RT, diluted with water and the resulting precipitate was collected by filtration by means of a Gooch funnel and washed with water, affording the title compound (0.61 g, 1.82 mmol, 66% yield) that was used in the next step without further purification.

LC-MS (ESI): m/z (M+1): 335.2 (Method 1)

### Intermediate 4: 2′-chloro-6′-(5-chloro-2-fluorophenyl)-3′-(methoxymethoxy)-3,4′-bipyridine

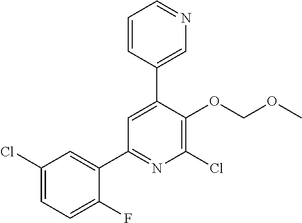 [SMILES: COCOc1c(-c2cccnc2)cc(-c2cc(Cl)ccc2F)nc1Cl]

To an ice-cooled solution of Intermediate 3 (0.61 g, 1.82 mmol) in dry DMF (2.9 mL), potassium tert-butoxide (0.51 g, 4.55 mmol) was added. After 15 min, chloromethyl methyl ether (0.32 mL, 4.19 mmol) was added dropwise. The mixture was further stirred at 0° C. for 1 h. Potassium tert-butoxide (0.14 g, 1.27 mmol) and chloromethyl methyl ether (0.07 mL, 0.91 mmol) were added again and the mixture was stirred for 30 min at 0° C. Cold water was added, followed by EtOAc, then the mixture was transferred to a separatory funnel. The organic phase was washed with sat. aqueous NaHCO₃ solution and brine, filtered through a phase separator and concentrated under vacuum. The crude material was purified by FC on Biotage silica gel (from 10% to 50% of EtOAc in c-Hex as eluent) to provide the title compound (0.52 g, 1.37 mmol, 75% yield).

LC-MS (ESI): m/z (M+1): 379.2 (Method 1)

### Intermediate 5: 6′-(5-chloro-2-fluorophenyl)-N-[(2,4-dimethoxyphenyl)methyl]-3′-(methoxymethoxy)-[3,4′-bipyridin]-2′-amine

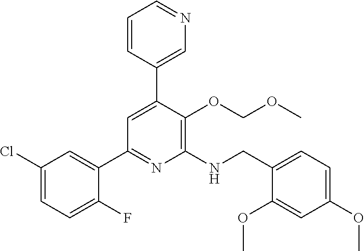 [SMILES: COCOc1c(-c2cccnc2)cc(-c2cc(Cl)ccc2F)nc1NCc1ccc(OC)cc1OC]

In a suitable vial, a mixture of Intermediate 4 (50.0 mg, 0.13 mmol), Pd(dba)₂ (7.6 mg, 0.01 mmol), sodium tert-butoxide (15.8 mg, 0.16 mmol) and DTBPF (7.4 mg, 0.02 mmol) was suspended in 1,4-dioxane (0.53 mL). The vessel was sealed, evacuated and backfilled with N₂ (3×), then 2,4-dimethoxybenzylamine (39.6 μL, 0.26 mmol) was added. The reaction was heated at 100° C. for 30 min. The mixture was filtered through a Celite® pad, washing the pad with EtOAc. The filtrate was washed with brine (2×). The organic phase was filtered through a phase separator and concentrated under vacuum. The crude material was purified by FC on Biotage silica gel (from 10% to 50% of EtOAc in c-Hex as eluent), affording the title compound (48 mg, 0.09 mmol, 71% yield) as pale orange oil.

LC-MS (ESI): m/z (M+1): 510.1 (Method 1)

### Intermediate 6: 2′-amino-6′-(5-chloro-2-fluorophenyl)-[3,4′-bipyridin]-3′-ol

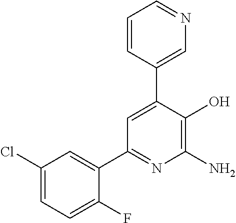 [SMILES: Nc1nc(-c2cc(Cl)ccc2F)cc(-c2cccnc2)c1O]

Intermediate 5 (46.0 mg, 0.09 mmol) was dissolved in a mixture of DCM and TFA (2 mL, 4:1 ratio). The reaction was stirred at RT for 30 min. Toluene (2 mL) was added and the solvents were removed under reduced pressure. The residue material was charged on SCX cartridge (1 g), that was washed with MeOH, and eluted with 1 N NH3 in MeOH, affording the title compound (35 mg, recovery assumed quantitative) as orange oil, used for next step without further purification.

LC-MS (ESI): m/z (M+1): 316.1 (Method 1)

### Intermediate 7: 2-chloro-6-(5-chloro-2-fluorophenyl)-4-(isoquinolin-4-yl)pyridin-3-ol

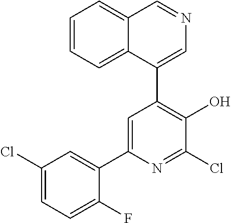 [SMILES: Oc1c(-c2cncc3ccccc23)cc(-c2cc(Cl)ccc2F)nc1Cl]

Prepared from Intermediate 2 (500 mg, 1.30 mmol) and 4-isoquinolineboronic acid (451 mg, 2.6 mmol), following similar procedure as for Intermediate 3. Title compound (114 mg, 0.30 mmol, 23% yield) was obtained as yellow solid.

LC-MS (ESI): m/z (M+1): 385.0 (Method 1)

### Intermediate 8: 4-[2-chloro-6-(5-chloro-2-fluorophenyl)-3-(methoxymethoxy)pyridin-4-yl]isoquinoline

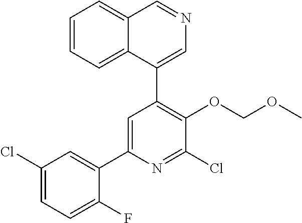 [SMILES: COCOc1c(-c2cncc3ccccc23)cc(-c2cc(Cl)ccc2F)nc1Cl]

Prepared from Intermediate 7 (87.0 mg, 0.23 mmol), following similar procedure as for Intermediate 4. Title compound (78 mg, 0.18 mmol, 80% yield) was obtained as colorless oil that solidifies upon cooling.

LC-MS (ESI): m/z (M+1): 429.1 (Method 1)

### Intermediate 9: 6-(5-chloro-2-fluorophenyl)-N-[(2,4-dimethoxyphenyl)methyl]-4-(isoquinolin-4-yl)-3-(methoxymethoxy)pyridin-2-amine

Prepared from Intermediate 8 (162 mg, 0.38 mmol), following similar procedure as

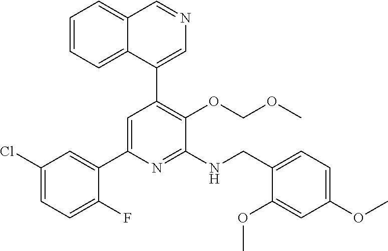 [SMILES: COCOc1c(-c2cncc3ccccc23)cc(-c2cc(Cl)ccc2F)nc1NCc1ccc(OC)cc1OC]

for Intermediate 5. Title compound (166 mg, 0.296 mmol, 79% yield) was obtained as pale yellow oil.

LC-MS (ESI): m/z (M+1): 560.1 (Method 1)

### Intermediate 10: 2-amino-6-(5-chloro-2-fluorophenyl)-4-(isoquinolin-4-yl)pyridin-3-ol

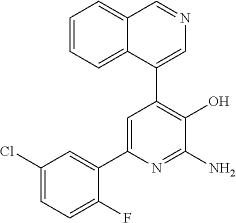 [SMILES: Nc1nc(-c2cc(Cl)ccc2F)cc(-c2cncc3ccccc23)c1O]

Prepared from Intermediate 9 (162 mg, 0.29 mmol), following similar procedure as for Intermediate 6. Title compound (130 mg, recovery assumed quantitative) was obtained as orange solid.

LC-MS (ESI): m/z (M+1): 366.1 (Method 1)

### Intermediate 11: 4-bromo-2-chloro-6-iodo-3-(methoxymethoxy)pyridine

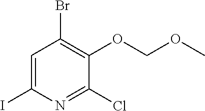

Intermediate 11 was prepared following the reaction methodology reported in J. Org. Chem. 2014, 79, 908-918. Under N₂ atmosphere, to a stirred solution of 2-chloro-6-iodo-pyridin-3-ol (3.0 g, 11.4 mmol) in DCM (70 mL), diisopropyl amine (2.56 mL, 18.2 mmol) and NBS (2.03 g, 11.4 mmol) were added. This mixture was stirred at RT for 3 hrs, before adding chloromethyl methyl ether (1.3 mL, 17.1 mmol) dropwise. The reaction was further stirred at RT for 2 hrs, then volatiles were removed under vacuum. The residue material was taken up with EtOAc and washed with water. The organic layer was separated, dried over Na₂SO₄, filtered and concentrated again. The crude material was purified by FC on Biotage silica gel (30% of EtOAc in c-Hex as eluent), to give the title compound (3.2 g, 8.5 mmol, 74% yield).

LC-MS (ESI): m/z (M+1): 377.8 (Method 1).

### Intermediate 12: 4-bromo-6-chloro-5-(methoxymethoxy)-6′-methyl-2,2′-bipyridine

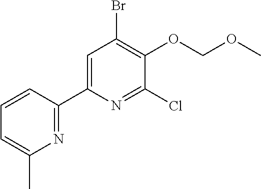 [SMILES: COCOc1c(Br)cc(-c2cccc(C)n2)nc1Cl]

In a suitable vial, Pd(PPh₃)₄ (147 mg, 0.13 mmol) was added to a stirred solution of Intermediate 11 (480 mg, 1.27 mmol) in dry toluene (7 mL) at RT. The mixture was degassed by N₂ bubbling before adding 2-methyl-6-(tributylstannyl)pyridine (485 mg, 1.27 mmol). The vial was sealed and heated at 100° C. for 6 hrs. Volatiles were removed under vacuum, the residue was material was suspended in a mixture of Et₂O and 1 N aqueous KF solution and vigorously stirred at RT for 2 hrs. The mixture was filtered through a Celite® pad, then the organic layer was separated, dried over Na₂SO₄ and evaporated at reduced pressure. The residue material was purified by FC on Biotage silica gel (10% of EtOAc in c-Hex as eluent) to give the title compound (200 mg, 0.58 mmol, 46% yield).

LC-MS (ESI): m/z (M+1): 342.9 (Method 2).

### Intermediate 13: 6-chloro-4-(isoquinolin-4-yl)-5-(methoxymethoxy)-6′-methyl-2,2′-bipyridine

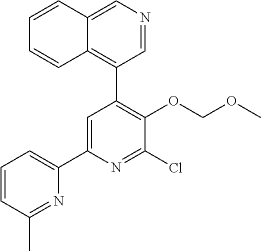 [SMILES: COCOc1c(-c2cncc3ccccc23)cc(-c2cccc(C)n2)nc1Cl]

In a suitable vial charged with Intermediate 12 (200 mg, 0.52 mmol), 4-isoauinolineboronic acid (109 mg, 0.63 mmol), Pd(PPh₃) (30.3 mg, 0.03 mmol) and NaHCO₃ (98 mg, 1.15 mmol), a mixture of toluene, ethanol and water (7 mL, 5:1:1 ratio) was added. The mixture was degassed by N₂ bubbling, then heated at 90° C. for 5 hrs. The mixture was cooled down to RT, diluted with EtOAc and washed with water. The organic layer was separated, dried over Na₂SO₄, filtered and evaporated under vacuum. The residue material was purified by FC on Biotage silica-NH gel (20% of EtOAc in c-Hex as eluent), affording the title compound (100 mg, 0.26 mmol, 49% yield).

LC-MS (ESI): m/z (M+1): 392.2 (Method 1).

### Intermediate 14: N-[(2,4-dimethoxyphenyl)methyl]-4-(isoquinolin-4-yl)-5-(methoxymethoxy)-6′-methyl-[2,2′-bipyridin]-6-amine

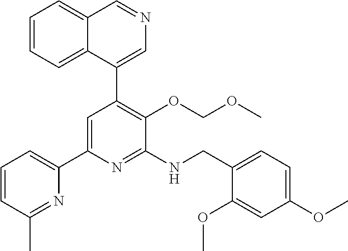 [SMILES: COCOc1c(-c2cncc3ccccc23)cc(-c2cccc(C)n2)nc1NCc1ccc(OC)cc1OC]

Title compound (100 mg, 0.19 mmol, 75% yield) was prepared from Intermediate 13 (100.0 mg, 0.260 mmol), following similar procedure as for Intermediate 5. LC-MS (ESI): m/z (M+1): 523.2 (Method 2).

### Intermediate 15: 6-amino-4-(isoquinolin-4-yl)-6′-methyl-[2,2′-bipyridin]-5-ol

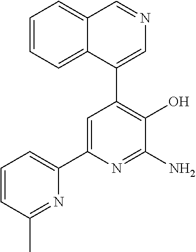 [SMILES: Cc1cccc(-c2cc(-c3cncc4ccccc34)c(O)c(N)n2)n1]

Prepared from Intermediate 14 (100 mg, 0.19 mmol), following similar procedure as for Intermediate 6. Title compound (50 mg, 0.15 mmol, 80% yield) was obtained after purification by FC on Biotage silica gel (from 0% to 5% of MeOH in DCM as eluent, 0.5% of aqueous ammonia as additive).

LC-MS (ESI): m/z (M+1): 329.1 (Method 2).

### Intermediate 16: 4-bromo-2-chloro-3-(methoxymethoxy)-6-(4-methyl-1,3-thiazol-2-yl)pyridine

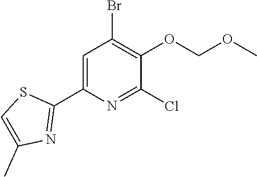 [SMILES: COCOc1c(Br)cc(-c2nc(C)cs2)nc1Cl]

In a suitable vial, a solution of Intermediate 11 (2.0 g, 4.49 mmol) and 4-methyl-2-(tributylstannyl)-1,3-thiazole (2.09 g, 5.39 mmol) in dry toluene (25 mL) was degassed by N₂ bubbling before adding Pd(PPh₃)₄ (519 mg, 0.45 mmol). The vial was sealed and heated at 100° C. for 45 min. Volatiles were removed under vacuum, the residue was material was suspended in a mixture of Et₂O and 1 N aqueous KF solution and vigorously stirred at RT for 3 hrs. The mixture was filtered through a Celite® pad, then the organic layer was separated, dried over Na₂SO₄ and evaporated at reduced pressure. The residue material was purified by FC on Biotage silica gel (from 0% to 10% of EtOAc in c-Hex as eluent) to give the title compound (720 mg, 2.06 mmol, 46% yield).

LC-MS (ESI): m/z (M+1): 348.9 (Method 2).

### Intermediate 17: 4-[2-chloro-3-(methoxymethoxy)-6-(4-methyl-1,3-thiazol-2-yl)pyridin-4-yl]isoquinoline

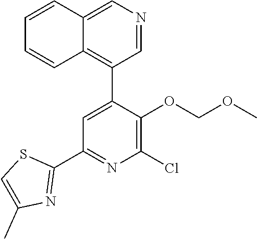 [SMILES: COCOc1c(-c2cncc3ccccc23)cc(-c2nc(C)cs2)nc1Cl]

Title compound (200 mg, 0.50 mmol, 25% yield) was prepared from 4-bromo-2-chloro-3-(methoxymethoxy)-6-(4-methyl-1,3-thiazol-2-yl)pyridine (Intermediate 16, 700 mg, 2.0 mmol) and 4-isoquinolineboronic acid (416 mg, 2.4 mmol) following similar procedure as for Intermediate 13.

LC-MS (ESI): m/z (M+1): 398.0 (Method 2).

### Intermediate 18: N-[(2,4-dimethoxyphenyl)methyl]-4-(isoquinolin-4-yl)-3-(methoxymethoxy)-6-(4-methyl-1,3-thiazol-2-yl)pyridin-2-amine

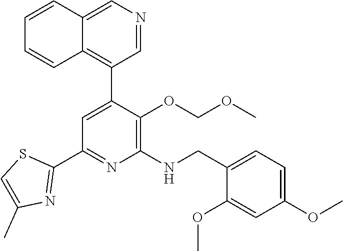 [SMILES: COCOc1c(-c2cncc3ccccc23)cc(-c2nc(C)cs2)nc1NCc1ccc(OC)cc1OC]

Title compound (100 mg, 0.19 mmol, 47% yield) was prepared from Intermediate 17 (160 mg, 0.40 mmol), following similar procedure as for Intermediate 5.

LC-MS (ESI): m/z (M+1): 529.2 (Method 2).

### Intermediate 19: 2-amino-4-(isoquinolin-4-yl)-6-(4-methyl-1,3-thiazol-2-yl)pyridin-3-ol

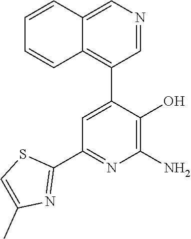 [SMILES: Cc1csc(-c2cc(-c3cncc4ccccc34)c(O)c(N)n2)n1]

Prepared from Intermediate 18 (100 mg, 0.19 mmol), following similar procedure as for Intermediate 6. Title compound (55 mg, 0.16 mmol, 87% yield) was obtained after purification by FC on Biotage silica gel (from 0% to 10% of MeOH in DCM as eluent, 0.5% of aqueous ammonia as additive).

LC-MS (ESI): m/z (M+1): 335.2 (Method 1).

### Intermediate 20: 6-(5-chloro-2-fluorophenyl)-2-nitropyridin-3-ol

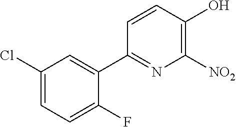 [SMILES: O=[N+]([O-])c1nc(-c2cc(Cl)ccc2F)ccc1O]

A mixture of 6-bromo-2-nitropyridin-3-ol (5.0 g, 22.8 mmol), 5-chloro-2-fluorophenylboronic acid (4.78 g, 27.4 mmol), Na₂CO₃ (7.26 g, 68.5 mmol) and Pd(dppf)Cl₂ DCM (0.93 g, 1.14 mmol) was dissolved in 1,4-dioxane/water (75 mL, 2:1 ratio). The mixture was degassed, then heated at 100° C. for 2.5 hrs. The reaction was diluted with EtOAc, filtered through a Celite® pad, washing with EtOAc. The filtrate was washed with acidified brine. The organic phase was filtered through a phase separator and evaporated under vacuum. The residue was purified by FC on Biotage silica gel (100% c-Hex, then 100% of DCM as eluent), affording the title compound, as yellow solid.

LC-MS (ESI): m/z (M+1): 269.0 (Method 1)

### Intermediate 21: 4-bromo-6-(5-chloro-2-fluorophenyl)-2-nitropyridin-3-ol

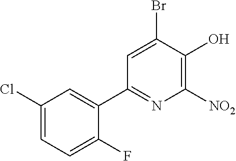 [SMILES: O=[N+]([O-])c1nc(-c2cc(Cl)ccc2F)cc(Br)c1O]

To an ice-cooled suspension of Intermediate 20 (5.49 g, 20.42 mmol) in dry MeOH (100 mL), sodium methoxide (˜25% solution in MeOH, 5.6 mL, 24.51 mmol) was added and the suspension turned into a yellow solution. Then molecular bromine (1.26 mL, 24.51 mmol) was added dropwise. The solution turned into a suspension and the mixture was stirred at 0° C. for 45 min. The mixture was quenched with water, then extracted with EtOAc (2×). The combined organic layers were washed with 10% aqueous Na₂S₂O₃ (2×) and brine (1×), filtered through a phase separator and concentrated under vacuum. The crude material was taken up with EtOAc and the resulting precipitate was filtered and dried at high vacuum to 4-bromo-6-(5-chloro-2-fluorophenyl)-2-nitropyridin-3-ol (4.03 g, 11.6 mmol, 57% yield), as an orange solid. The material was used in the next step without further purification.

LC-MS (ESI): m/z (M-1): 344.9 (Method 1)

### Intermediate 22: 2-amino-4-bromo-6-(5-chloro-2-fluorophenyl)pyridin-3-ol

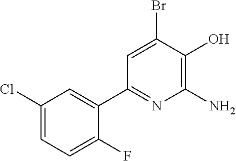 [SMILES: Nc1nc(-c2cc(Cl)ccc2F)cc(Br)c1O]

To an ice-cooled solution of Intermediate 21 (4.03 g, 11.6 mmol) in a mixture of dry MeOH and dry THE (100 mL, 1:1 ratio), nickel(II) chloride hexahydrate (0.56 g, 2.32 mmol) and sodium borohydride (0.88 g, 23.2 mmol) were subsequently added. The reaction was stirred at 0° C. for 30 min. The mixture was quenched with water and allowed to reach the RT. The pH was adjusted to 3-4 using 2 N HCl solution and the mixture was extracted with EtOAc (2×). The combined organic layers were washed with brine, filtered through a phase separator and evaporated under vacuum. The residue was dissolved in MeOH and loaded on a SCX cartridge (20 g), washed with MeOH then eluted with 2 N ammonia in MeOH. Proper fractions were evaporated to provide the title compound (2.97 g, 9.35 mmol, 81% yield) as a brown solid. The material was used in the next step without further purification.

LC-MS (ESI): m/z (M+1): 316.9 (Method 1)

### Intermediate 23: 4-bromo-6-(5-chloro-2-fluorophenyl)-3-(2-chloroethoxy)pyridin-2-amine

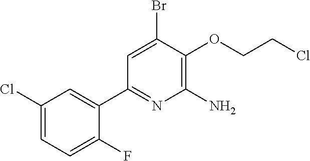 [SMILES: Nc1nc(-c2cc(Cl)ccc2F)cc(Br)c1OCCCl]

To a mixture of Intermediate 22 (2.97 g, 9.35 mmol) and K₂CO₃ (3.23 g, 23.4 mmol) in dry DMF (60 mL), 1-bromo-2-chloroethane (1.40 mL, 16.8 mmol) was added and the reaction was stirred at RT overnight. The mixture was diluted with EtOAc and washed with sat. aqueous NaHCO₃ solution (3×) and brine. The organic phase was filtered through a phase separator and concentrated under vacuum. The residue was purified by FC on Biotage Silica-NH gel (from 0% to 30% of EtOAc in c-Hex as eluent) to provide the title compound (2.19 g, 5.75 mmol, 62% yield).

LC-MS (ESI): m/z (M+1): 378.9 (Method 1)

### Intermediate 24: 8-bromo-6-(5-chloro-2-fluorophenyl)-2H,3H,4H-pyrido[3,2-b][1,4]oxazine

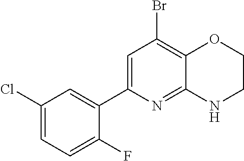 [SMILES: Fc1ccc(Cl)cc1-c1cc(Br)c2c(n1)NCCO2]

Under N₂ atmosphere, a solution of Intermediate 23 (2.19 g, 5.75 mmol) in dry DMF (50 mL) was added dropwise to an ice-cooled suspension of sodium hydride (60% dispersion in mineral oil, 299 mg, 7.48 mmol) in dry DMF (5 mL). A yellow color developed. The mixture was allowed to reach the RT and stirred for 30 min. The reaction was cooled again to 0° C., quenched with water and diluted with EtOAc. Phases were separated and the organic layer was washed with sat. aqueous NaHCO₃ solution (3×) and brine, filtered through a phase separator and concentrated under vacuum, affording the title compound (2.05 g, 5.96 mmol, recovery assumed quantitative) as an off-white solid.

LC-MS (ESI): m/z (M+1): 342.9 (Method 1)

### Intermediate 25: 5-[6-(5-chloro-2-fluorophenyl)-2H,3H,4H-pyrido[3,2-b][1,4]oxazin-8-yl]pyridine-3-carbonitrile

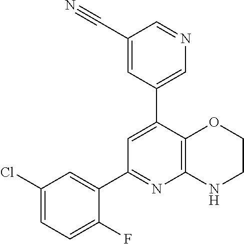 [SMILES: N#Cc1cncc(-c2cc(-c3cc(Cl)ccc3F)nc3c2OCCN3)c1]

In a suitable vial, a mixture of Intermediate 24 (60 mg, 0.17 mmol), 5-cyanopyridine-3-boronic acid (31.0 mg, 0.21 mmol), Na₂CO₃ (37.0 mg, 0.35 mmol) and Pd(dppf)Cl₂ DCM (7.15 mg, 0.01 mmol) was dissolved in 1,4-dioxane/water (2.2 mL, 3:1 ratio). The vessel was sealed, evacuated and backfilled with N₂ (3×), then heated at 80° C. for 45 min in a PLS. The reaction was diluted with EtOAc, filtered through a Celite® pad, washing with EtOAc. The filtrate was washed with acidified brine. The organic phase was filtered through a phase separator and evaporated under vacuum. The crude was purified by FC on Biotage silica gel (from 20% to 100% of EtOAc in c-Hex as eluent), affording the title compound (55 mg, 0.15 mmol, 88% yield).

LC-MS (ESI): m/z (M+1): 367.0 (Method 1)

### Intermediate 26: methyl 5-[6-(5-chloro-2-fluorophenyl)-2H,3H,4H-pyrido[3,2-b][1,4]oxazin-8-yl]pyridine-3-carboxylate

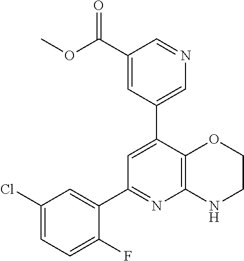 [SMILES: COC(=O)c1cncc(-c2cc(-c3cc(Cl)ccc3F)nc3c2OCCN3)c1]

Prepared from Intermediate 24 (50 mg, 0.15 mmol) and 3-(methoxycarbonyl)pyridine-5-boronic acid pinacol ester (46 mg, 0.17 mmol), following similar procedure as for Intermediate 25, affording the title compound (52 mg, 0.13 mmol, 89% yield) as white solid.

LC-MS (ESI): m/z (M+2): 200.6 (Method 1).

### Intermediate 27: N-(5-bromopyridin-3-yl)prop-2-enamide

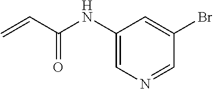

To an ice-cooled solution of 3-amino-5-bromopyridine (500 mg, 2.89 mmol) in dry DCM (20 mL), TEA (1.21 mL, 8.67 mmol) and acryloyl chloride (352 μL, 4.34 mmol) were added. The solution became an orange suspension that was stirred at 0° C. for 1 h. Acryloyl chloride (59 μL, 0.725 mmol) was added again and the reaction was stirred for 30 min at 0° C. Water was added, the phases were separated and the organic phase was filtered through a phase separator and evaporated under vacuum. The crude material was purified by FC on Biotage silica gel (from 30% to 90% of EtOAc in c-Hex as eluent), affording the title compound (415 mg, 1.83 mmol, 63% yield) as pale yellow solid.

LC-MS (ESI): m/z (M+1): 226.9 (Method 1).

### Intermediate 28: N-(5-bromopyridin-3-yl)-3-(dimethylamino)propanamide

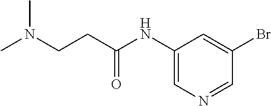

To a solution of Intermediate 27 (100 mg, 0.44 mmol) in THE (2 mL), dimethylamine (2 M solution in THF, 0.48 mL, 0.97 mmol) was added and the mixture heated at 60° C. for 1 h in a PLS. Volatiles were removed under vacuum and the residue material was purified by FC on Biotage silica-NH gel (from 5% to 30% of EtOAc in c-Hex as eluent), affording the title compound (100 mg, 0.37 mmol, 83% yield) as white solid.

LC-MS (ESI): m/z (M+1): 272.1 (Method 1).

Intermediate 29: 3-(dimethylamino)-N-[5-(4,4,5,5-tetramethyl-1,3,2-dioxaborolan-2-yl)pyridin-3-yl]propanamide

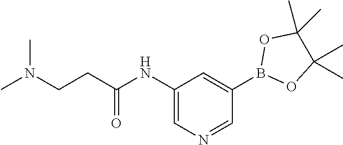

A mixture of Intermediate 28 (50 mg, 0.18 mmol), bis(pinacolato)diboron (56 mg, 0.22 mmol), KOAc (54.1 mg, 0.55 mmol) and Pd(dppf)Cl₂ DCM (7.5 mg, 0.01 mmol) was suspended in 1,4-dioxane (1 mL). The vessel was sealed, evacuated and back-filled with N₂ (3×), then heated at 100° C. for 1 h. The mixture was diluted with EtOAc, filtered through a Celite® pad, washing with EtOAc. The solvents were removed under vacuum to give the title compound (139 mg, recovery assumed quantitative) as brownish solid, that was used in the next step without further purification.

LC-MS (ESI): m/z (boronic acid M+1): 238.1 (Method 1).

### Intermediate 30: N-(5-bromopyridin-3-yl)-3-(morpholin-4-yl)propanamide

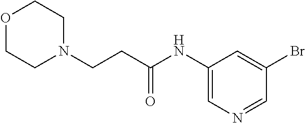 [SMILES: O=C(CCN1CCOCC1)Nc1cncc(Br)c1]

Prepared from Intermediate 27 (150 mg, 0.66 mmol) and morpholine (0.13 mL, 1.45 mmol), following similar procedure as for Intermediate 28, affording the title compound (175 mg, 0.56 mmol, 84% yield) as an off-white solid.

LC-MS (ESI): m/z (M+1): 314.0 (Method 1).

### Intermediate 31: 3-(morpholin-4-yl)-N-[5-(4,4,5,5-tetramethyl-1,3,2-dioxaborolan-2-yl)pyridin-3-yl]propenamide

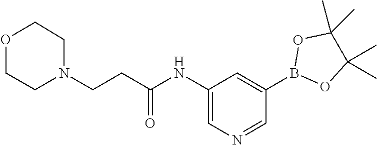 [SMILES: CC1(C)OB(c2cncc(NC(=O)CCN3CCOCC3)c2)OC1(C)C]

Prepared from Intermediate 30 (57.8 mg, 0.18 mmol), following similar procedure as for Intermediate 29, affording the title compound (136 mg, recovery assumed quantitative) as brownish solid, that was used in the next step without purification.

LC-MS (ESI): m/z (boronic acid M+1): 280.2 (Method 1).

### Intermediate 32: 5-[6-(5-chloro-2-fluorophenyl)-2H,3H,4H-pyrido[3,2-b][1,4]oxazin-8-yl]pyridine-3-carboxylic Acid

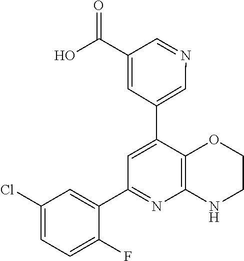 [SMILES: O=C(O)c1cncc(-c2cc(-c3cc(Cl)ccc3F)nc3c2OCCN3)c1]

Intermediate 26 (300 mg, 0.75 mmol) and lithium hydroxide monohydrate (63 mg, 1.5 mmol) were suspended in THF/water (10 mL, 4:1 ratio) and stirred at RT overnight. The residue was taken up with water, the pH adjusted to 5-6 using 2 N HCl solution and the resulting yellow precipitate was filtered and washed with water affording the title compound (290 mg, 0.75 mmol, quantitative yield).

LC-MS (ESI): m/z (M+1): 386.1 (Method 1)

### Intermediate 33: N-(5-bromopyridin-3-yl)-3-(pyrrolidin-1-yl)propanamide

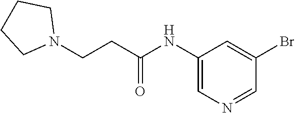 [SMILES: O=C(CCN1CCCC1)Nc1cncc(Br)c1]

Prepared from Intermediate 27 (110 mg, 0.48 mmol) and pyrrolidine (89 μL, 1.07 mmol), following similar procedure as for Intermediate 28, affording the title compound (102 mg, 0.34 mmol, 71% yield) as an off-white solid.

LC-MS (ESI): m/z (M+1): 298.1 (Method 1).

### Intermediate 34: 3-(pyrrolidin-1-yl)-N-[5-(4,4,5,5-tetramethyl-1,3,2-dioxaborolan-2-yl)pyridin-3-yl]propanamide

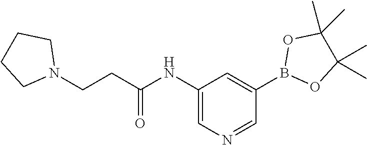 [SMILES: CC1(C)OB(c2cncc(NC(=O)CCN3CCCC3)c2)OC1(C)C]

Prepared from Intermediate 33 (54.9 mg, 0.18 mmol), following similar procedure as for Intermediate 29, affording the title compound (126 mg recovery assumed quantitative) as brownish solid, that was used in the next step without purification.

LC-MS (ESI): m/z (boronic acid M+1): 264.1 (Method 1).

### Intermediate 35: N-(5-bromopyridin-3-yl)-2-chloroacetamide

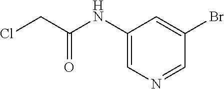 [SMILES: O=C(CCl)Nc1cncc(Br)c1]

To an ice-cooled solution of 3-amino-5-bromopyridine (500 mg, 2.89 mmol) in dry DCM (20 mL), TEA (1.21 mL, 8.67 mmol) and chloroacetyl chloride (345 μL, 4.34 mmol) were added. The solution turned into an orange suspension that was stirred at 0° C. for 1 h. Water was added, the phases were separated and the organic phase was filtered through a phase separator and evaporated under vacuum. The crude material was purified by FC on Biotage silica gel (from 30% to 75% of EtOAc in c-Hex as eluent), affording the title compound (625 mg, 2.51 mmol, 87% yield) as a pale yellow solid.

LC-MS (ESI): m/z (M+1): 248.9 (Method 1).

### Intermediate 36: N-(5-bromopyridin-3-yl)-2-(dimethylamino)acetamide

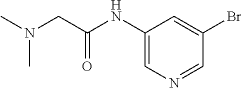

To a suspension of Intermediate 35 (150 mg, 0.60 mmol) and K₂CO₃ (249 mg, 1.80 mmol) in DMF (3.0 mL), dimethylamine (2 M solution in THF, 0.6 mL, 1.2 mmol) was added and the reaction was shaken at RT in a PLS overnight. Solids were filtered-off and the filtrate was concentrated under vacuum. The residue material was purified by FC on Biotage silica-NH gel (from 5% to 75% of EtOAc in c-Hex as eleuent), to give the title compound (153 mg, 0.59 mmol, 98% yield) as pale yellow oil that solidifies upon cooling.

LC-MS (ESI): m/z (M+1): 258.0 (Method 1).

### Intermediate 37: 2-(dimethylamino)-N-[5-(4,4,5,5-tetramethyl-1,3,2-dioxaborolan-2-yl)pyridin-3-yl]acetamide

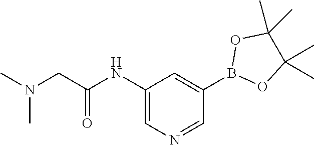

A mixture of Intermediate 36 (47.5 mg, 0.18 mmol), bis(pinacolato)diboron (56.1 mg, 0.22 mmol), KOAc (54.2 mg, 0.55 mmol) and Pd(dppf)Cl₂ DCM (7.53 mg, 0.01 mmol) was suspended in 1,4-dioxane (1 mL). The vessel was sealed, evacuated and back-filled with N₂ (3×), then heated at 120° C. for 2 hrs. The mixture was diluted with EtOAc, filtered through a Celite® pad, washing with EtOAc. The solvents were removed under vacuum to give the title compound (126 mg, recovery assumed quantitative) as brownish solid, that was used in the next step without further purification.

LC-MS (ESI): m/z (boronic acid M+1): 224.1 (Method 1).

### Intermediate 38: Tert-Butyl 4-{2-[(5-bromopyridin-3-yl)carbamoyl]ethyl}piperazine-1-carboxylate

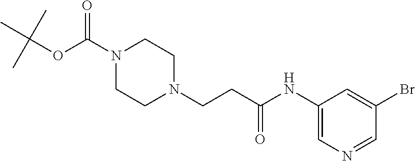 [SMILES: CC(C)(C)OC(=O)N1CCN(CCC(=O)Nc2cncc(Br)c2)CC1]

Prepared from Intermediate 27 (150 mg, 0.66 mmol) and tert-butyl piperazine-1-carboxylate (271 mg, 1.45 mmol), following similar procedure as for Intermediate 28, affording the title compound (315 mg, recovery assumed quantitative) as an off-white solid.

LC-MS (ESI): m/z (M+1): 413.2 (Method 1).

### Intermediate 39: Tert-Butyl 4-(2-{[5-(4,4,5,5-tetramethyl-1,3,2-dioxaborolan-2-yl)pyridin-3-yl]carbamoyl}ethyl)piperazine-1-carboxylate

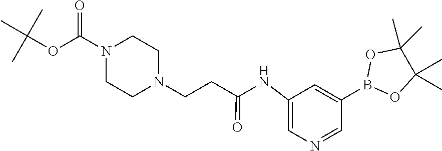 [SMILES: CC(C)(C)OC(=O)N1CCN(CCC(=O)Nc2cncc(B3OC(C)(C)C(C)(C)O3)c2)CC1]

Prepared from Intermediate 38 (77.5 mg, 0.19 mmol), following similar procedure as for Intermediate 29, affording the title compound (176 mg, recovery assumed quantitative) as brownish solid, that was used in the next step without purification.

LC-MS (ESI): m/z (boronic acid M+1): 379.3 (Method 1).

### Intermediate 40: tert-butyl 4-[2-({5-[6-(5-chloro-2-fluorophenyl)-2H,3H,4H-pyrido[3,2-b][1,4]oxazin-8-yl]pyridin-3-yl}carbamoyl)ethyl]piperazine-1-carboxylate

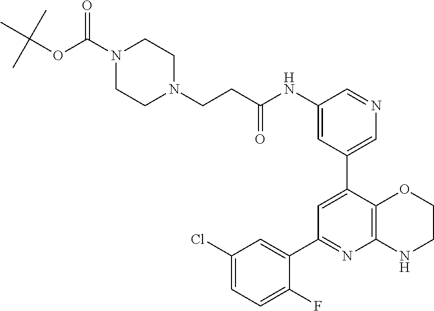 [SMILES: CC(C)(C)OC(=O)N1CCN(CCC(=O)Nc2cncc(-c3cc(-c4cc(Cl)ccc4F)nc4c3OCCN4)c2)CC1]

Prepared from Intermediate 24 (40 mg, 0.12 mmol) and tert-butyl 4-(2-{[5-(4,4,5,5-tetramethyl-1,3,2-dioxaborolan-2-yl)pyridin-3-yl]carbamoyl}ethyl)piperazine-1-carboxylate (Intermediate 39, 0.19 theoretical mmol), following similar procedure as for Intermediate 25, affording the title compound (58 mg, 0.10 mmol, 83% yield) as pale yellow oil.

LC-MS (ESI): m/z (M+2): 299.3 (Method 1).

### Intermediate 41: N-(5-bromopyridin-3-yl)-4-chlorobutanamide

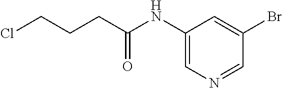 [SMILES: O=C(CCCCl)Nc1cncc(Br)c1]

To an ice-cooled solution of 3-amino-5-bromopyridine (500 mg, 2.89 mmol) and TEA (1.21 mL, 8.67 mmol) in dry DCM (20 mL), 4-chlorobutyryl chloride (356 μL, 3.18 mmol) was added dropwise and the reaction was stirred at 0° C. for 30 min. 4-chlorobutanoyl chloride (97 μl, 0.87 mmol) was added again and the reaction was further stirred for 30 min. Water was added, the phases were separated and the organic phase was filtered through a phase separator and evaporated under vacuum. The crude material was purified by FC on Biotage silica gel (from 5% to 60% of EtOAc in c-Hex as eluent), affording the title compound (714 mg, 2.57 mmol, 89% yield) as orange oil that solidifies upon cooling.

LC-MS (ESI): m/z (M+1): 277.1 (Method 1).

### Intermediate 42: N-(5-bromopyridin-3-yl)-4-(dimethylamino)butanamide

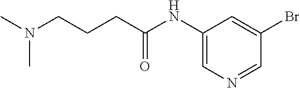 [SMILES: CN(C)CCCC(=O)Nc1cncc(Br)c1]

A solution of Intermediate 41 (150 mg, 0.54 mmol) and dimethylamine (2 M solution in THF, 0.81 mL, 1.62 mmol) in THE (5.5 mL) was shaken at 60° C. in a PLS, for 48 hrs. Volatiles were removed under vacuum and the residue material was purified by FC on Biotage silica-NH gel (from 10% to 100% of EtOAc in c-Hex as eleuent, then 10% of MeOH as additive), to the title compound (137 mg, 0.48 mmol, 89% yield) as pale yellow oil that slowly solidifies.

LC-MS (ESI): m/z (M+1): 286.2 (Method 1).

### Intermediate 43: 4-(dimethylamino)-N-[5-(4,4,5,5-tetramethyl-1,3,2-dioxaborolan-2-yl)pyridin-3-yl]butanamide

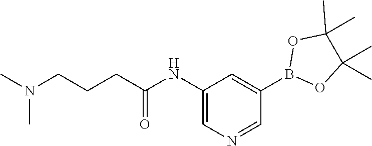

Prepared from Intermediate 42 (71.5 mg, 0.25 mmol), following similar procedure as for Intermediate 29, affording the title compound (190 mg, recovery assumed quantitative) as brownish solid, that was used in the next step without purification.

LC-MS (ESI): m/z (boronic acid M+1): 252.2 (Method 1).

### Intermediate 44: 2-amino-4-chloro-6-(5-chloro-2-fluorophenyl)pyridin-3-ol

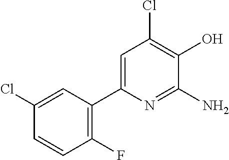 [SMILES: Nc1nc(-c2cc(Cl)ccc2F)cc(Cl)c1O]

To an ice-cooled solution of Intermediate 21 (3.33 g, 9.58 mmol) in a mixture of dry MeOH and dry THE (96 mL, 1:1 ratio), nickel(II) chloride hexahydrate (0.46 g, 1.92 mmol) and sodium borohydride (0.73 g, 19.15 mmol) were subsequently added. The reaction was stirred at 0° C. for 30 min. The mixture was quenched with water, diluted with EtOAc and washed with acidified brine. The organic phase was filtered through a phase separator and the solvents were removed under reduced pressure. The residue was dissolved in MeOH and loaded on a SCX column (10 g), washed with MeOH, then eluted with 2 N ammonia in MeOH. Proper fractions were evaporated to provide 2-amino-4-chloro-6-(5-chloro-2-fluorophenyl)pyridin-3-ol (3.0 g, recovery assumed quantitative). The conversion of the intended product (2-amino-4-bromo-6-(5-chloro-2-fluorophenyl)pyridin-3-ol), described above as Intermediate 22, into the title compound, occurred during the work-up procedure.

LC-MS (ESI): m/z (M+1): 273 (Method 1)

### Intermediate 45: 4-chloro-6-(5-chloro-2-fluorophenyl)-3-(2-chloroethoxy)pyridin-2-amine

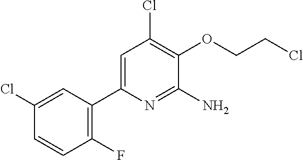 [SMILES: Nc1nc(-c2cc(Cl)ccc2F)cc(Cl)c1OCCCl]

Prepared from Intermediate 44 (3.0 g, 10.99 mmol), following the procedure described for Intermediate 23. Title compound (1.78 g, 5.3 mmol, 48% yield) was obtained as white solid.

LC-MS (ESI): m/z (M+1): 335.1 (Method 1).

### Intermediate 46: 8-chloro-6-(5-chloro-2-fluorophenyl)-2H,3H,4H-pyrido[3,2-b][1,4]oxazine

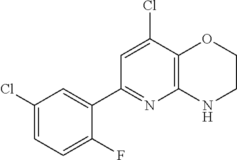 [SMILES: Fc1ccc(Cl)cc1-c1cc(Cl)c2c(n1)NCCO2]

Under N₂ atmosphere, to an ice-cooled suspension sodium hydride (60% dispersion in oil 276 mg, 6.9 mmol) in dry DMF (4.5 mL), a solution of Intermediate 45 (1.78 g, 5.3 mmol) in dry DMF (40 mL) was added dropwise. The reaction was allowed to warm to RT and stirred for 30 min. After cooling again to 0° C., water was added and the resulting white precipitate was collected by means of a Gooch funnel, washed with water and dried under vacuum, affording the title compound (1.49 g, 4.98 mmol, 94% yield) as white solid.

LC-MS (ESI): m/z (M+1): 299.1 (Method 1)

### Intermediate 47: N-(5-bromopyridin-3-yl)-3-(piperidin-1-yl)propanamide

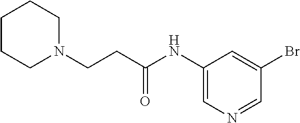 [SMILES: O=C(CCN1CCCCC1)Nc1cncc(Br)c1]

Prepared from Intermediate 27 (150 mg, 0.66 mmol) and piperidine (144 μL, 1.45 mmol), following similar procedure as for Intermediate 28, affording the title compound (180 mg, 0.58 mmol, 87% yield) as an off-white solid.

LC-MS (ESI): m/z (M+1): 312.1 (Method 1).

### Intermediate 48: 3-(piperidin-1-yl)-N-[5-(4,4,5,5-tetramethyl-1,3,2-dioxaborolan-2-yl)pyridin-3-yl]propanamide

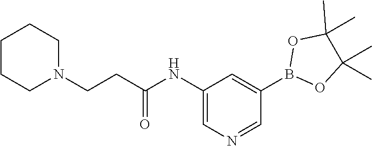 [SMILES: CC1(C)OB(c2cncc(NC(=O)CCN3CCCCC3)c2)OC1(C)C]

Prepared from Intermediate 47 (58 mg, 0.19 mmol), following similar procedure as for Intermediate 29, affording the title compound (140 mg, recovery assumed quantitative) as brownish solid, that was used in the next step without purification.

LC-MS (ESI): m/z (boronic acid M+1): 278.2 (Method 1).

### Intermediate 49: tert-butyl 4-[2-({5-[6-(5-chloro-2-fluorophenyl)-2H,3H,4H-pyrido[3,2-b][1,4]oxazin-8-yl]pyridin-3-yl}formamido)ethyl]piperazine-1-carboxylate

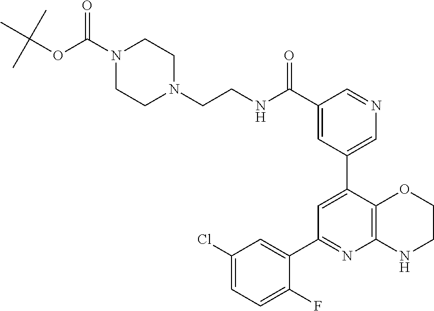 [SMILES: CC(C)(C)OC(=O)N1CCN(CCNC(=O)c2cncc(-c3cc(-c4cc(Cl)ccc4F)nc4c3OCCN4)c2)CC1]

A mixture of Intermediate 32 (50 mg, 0.13 mmol) and HATU (59.1 mg, 0.16 mmol) was suspended in dry DMF (2.7 mL), then DIPEA (67.7 μL, 0.39 mmol) was added and the mixture was stirred at RT for 15 min. tert-butyl 4-(2-aminoethyl)piperazine-1-carboxylate (44.6 mg, 0.19 mmol) was added and the reaction was further stirred at RT for 1 h. The mixture was diluted with EtOAc and washed with sat. aqueous NaHCO₃ solution (3×) and brine. The organic phase was filtered through a phase separator and concentrated under vacuum. The crude was purified by FC on Biotage silica-NH gel (from 20% to 100% of EtOAc in c-Hex, followed by 10% of MeOH in EtOAc), affording the title compound (53 mg, 0.09 mmol, 69% yield) as pale yellow foam.

LC-MS (ESI): m/z (M+2): 299.3 (Method 1).

### Intermediate 50: N-(5-bromopyridin-3-yl)-3-(methylamino)propanamide

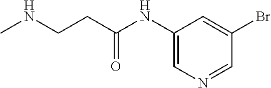

Prepared from Intermediate 27 (250 mg, 1.1 mmol) and methylamine (2.0 M solution in THF, 1.1 mL, 2.2 mmol), following similar procedure as for Intermediate 28, affording the title compound (282 mg, 1.09 mmol, 99%) as an off-white solid.

LC-MS (ESI): m/z (M+1): 258.1 (Method 2).

### Intermediate 51: tert-butyl N-{2-[(5-bromopyridin-3-yl)carbamoyl]ethyl}-N-methylcarbamate

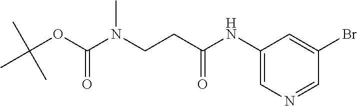 [SMILES: CN(CCC(=O)Nc1cncc(Br)c1)C(=O)OC(C)(C)C]

To a solution of Intermediate 50 (282 mg, 1.09 mmol) and TEA (0.3 mL, 2.19 mmol) in DCM (5.5 mL), di-tert-butyl dicarbonate (286 mg, 1.31 mmol) was added. The reaction was stirred at RT for 1 h. Volatiles were removed under vacuum and the crude material was purified by FC on Biotage silica-NH (from 0% to 50% of EtOAc in c-Hex as eluent), obtaining the title compound (204 mg, 0.57 mmol, 52%) as yellow oil that slowly solidifies.

LC-MS (ESI): m/z (M+1): 358.1 (Method 1).

### Intermediate 52: tert-butyl N-methyl-N-(2-{[5-(4,4,5,5-tetramethyl-1,3,2-dioxaborolan-2-yl)pyridin-3-yl]carbamoyl}ethyl)carbamate

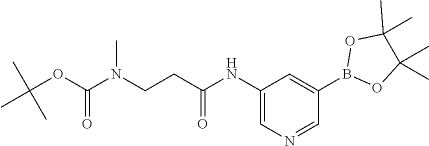 [SMILES: CN(CCC(=O)Nc1cncc(B2OC(C)(C)C(C)(C)O2)c1)C(=O)OC(C)(C)C]

Prepared from Intermediate 51 (110 mg, 0.310 mmol), following similar procedure as for Intermediate 29, affording the title compound (290 mg, recovery assumed quantitative) as brownish solid, that was used in the next step without purification.

LC-MS (ESI): m/z (boronic acid M+1): 324.2 (Method 1).

### Intermediate 53: tert-butyl N-[2-({5-[6-(5-chloro-2-fluorophenyl)-2H,3H,4H-pyrido[3,2-b][1,4]oxazin-8-yl]pyridin-3-yl}carbamoyl)ethyl]-N-methylcarbamate

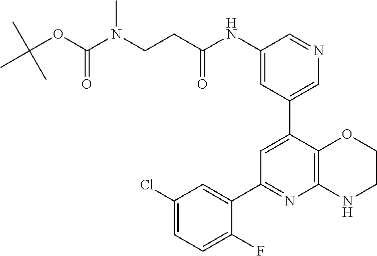 [SMILES: CN(CCC(=O)Nc1cncc(-c2cc(-c3cc(Cl)ccc3F)nc3c2OCCN3)c1)C(=O)OC(C)(C)C]

Prepared from Intermediate 24 (69.4 mg, 0.20 mmol) and Intermediate 52 (0.31 theoretical mmol), following similar procedure as for Intermediate 25, affording the title compound (81 mg, 0.15 mmol, 75% yield) as pale yellow oil.

LC-MS (ESI): m/z (M+1): 542.4 (Method 1).

### Intermediate 54: tert-butyl N-[2-({5-[6-(5-chloro-2-fluorophenyl)-2H,3H,4H-pyrido[3,2-b][1,4]oxazin-8-yl]pyridin-3-yl}formamido)ethyl]-N-methylcarbamate

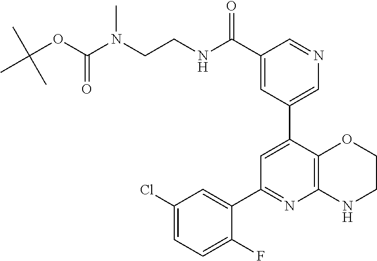 [SMILES: CN(CCNC(=O)c1cncc(-c2cc(-c3cc(Cl)ccc3F)nc3c2OCCN3)c1)C(=O)OC(C)(C)C]

Prepared from Intermediate 32 (50 mg, 0.13 mmol) and tert-butyl N-(2-aminoethyl)-N-methylcarbamate (35 μL, 0.19 mmol), following similar procedure as for Intermediate 49, affording the title compound (50 mg, 0.09 mmol, 71% yield) as pale yellow oil.

LC-MS (ESI): m/z (M+1): 542.4 (Method 1).

### Intermediate 55: {3-[(5-bromopyridin-3-yl)oxy]propyl}dimethylamine

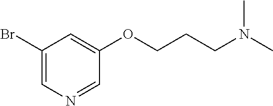

3-Bromo-5-hydroxypyridine (0.30 g, 1.72 mmol), K₂CO₃ (1.43 g, 10.3 mmol) and 3-dimethylamino-1-propyl chloride hydrochloride (0.82 g, 5.17 mmol) were suspended in DMF (7.0 mL). The mixture was heated at 80° C. and shaken through a PLS for 2 hrs. The reaction was diluted with EtOAc and sat. aqueous NaHCO₃, the phases were separated and the aqueous phase was extracted with EtOAc (2×). The combined organic layers were filtered through a phase separator and concentrated under vacuum. The crude material was dissolved in MeOH and loaded on a SCX column (10 g), washed with MeOH then eluted with 2 N ammonia in MeOH. Basic fractions were evaporated to give the title compound (285 mg, 1.10 mmol, 64% yield) as yellow oil.

LC-MS (ESI): m/z (M+1): 259.1 (Method 2).

### Intermediate 56: dimethyl(3-{[5-(4,4,5,5-tetramethyl-1,3,2-dioxaborolan-2-yl)pyridin-3-yl]oxy}propyl)amine

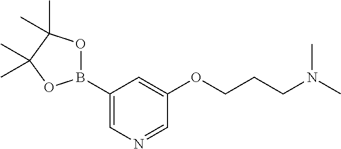

Prepared from Intermediate 55 (130 mg, 0.50 mmol), following similar procedure as for Intermediate 29, affording the title compound (310 mg, recovery assumed quantitative) as brownish solid, that was used in the next step without purification.

LC-MS (ESI): m/z (boronic acid M+1): 225.2 (Method 1).

### Intermediate 57: tert-butyl 4-[3-({5-[6-(5-chloro-2-fluorophenyl)-2H,3H,4H-pyrido[3,2-b][1,4]oxazin-8-yl]pyridin-3-yl}formamido)propyl]piperazine-1-carboxylate

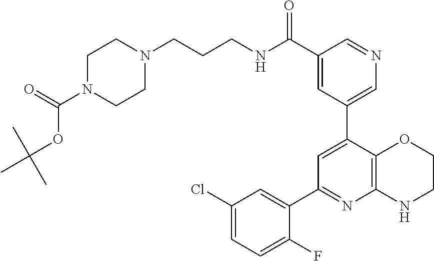

Prepared from Intermediate 32 (50 mg, 0.13 mmol) and tert-butyl 4-(3-aminopropyl)piperazine-1-carboxylate (47.3 mg, 0.19 mmol), following similar procedure as for Intermediate 49, affording the title compound (62 mg, 0.10 mmol, 78% yield) as pale yellow oil.

LC-MS (ESI): m/z (M+1): 611.5 (Method 1).

### Intermediate 58: 5-bromo-N-[3-(dimethylamino)propyl]pyridin-3-amine

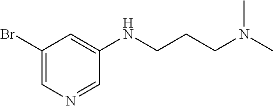

In a suitable vial, a mixture of 3,5-dibromopyridine (350 mg, 1.48 mmol), 3-(dimethylamino)-1-propylamine (186 μL, 1.48 mmol), Pd₂(dba)₃ (67.7 mg, 0.07 mmol), BINAP (92.0 mg, 0.15 mmol) and sodium tert-butoxide (175 mg, 1.85 mmol) was suspended in 1,4-dioxane (5.9 mL). The vial was sealed, evacuated and backfilled with N₂ (3×), then heated at 100° C. in a PLS for 1 h. The mixture was filtered through a Celite® pad, washing the pad with EtOAc. The organic phase was washed with sat. aqueous NaHCO₃ (3×) and brine (1×), filtered through a phase separator and concentrated under vacuum. The crude was purified by FC on Biotage silica-NH gel (from 0% to 60% of EtOAc in c-Hex as eluent), to give the title compound (190 mg, 0.74 mmol, 50% yield) as orange oil, that slowly solidifies.

¹H NMR (400 MHz, Chloroform-d) δ ppm 7.95 (d, J=1.98 Hz, 1H), 7.90 (d, J=2.64 Hz, 1H), 6.99 (t, J=2.20 Hz, 1H), 3.18 (t, J=6.38 Hz, 2H), 2.43 (t, J=6.38 Hz, 2H), 2.26 (s, 6H), 1.74-1.83 (m, 2H).

### Intermediate 59: N-[3-(dimethylamino)propyl]-5-(4,4,5,5-tetramethyl-1,3,2-dioxaborolan-2-yl)pyridin-3-amine

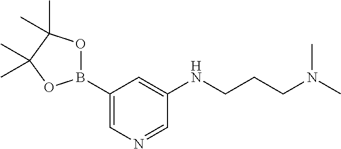

Prepared from Intermediate 58 (90.6 mg, 0.35 mmol), following similar procedure as for Intermediate 37, affording the title compound (230 mg, recovery assumed quantitative) as brownish solid, that was used in the next step without purification.

LC-MS (ESI): m/z (boronic acid M+1): 224.4 (Method 2).

### Intermediate 60: N-(5-bromopyridin-3-yl)-4-(methylamino)butanamide

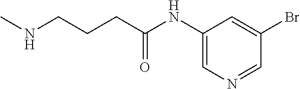

Prepared from Intermediate 41 (255 mg, 0.92 mmol) and methylamine (2.0 M solution in THF, 6.4 mL, 12.8 mmol), following the procedure described for Intermediate 42. The title compound (210 mg, 0.77 mmol, 84% yield) was obtained as yellow oil.

LC-MS (ESI): m/z (M+1): 272.1 (Method 1).

### Intermediate 61: Tert-Butyl N-{3-[(5-bromopyridin-3-yl)carbamoyl]propyl}-N-methylcarbamate

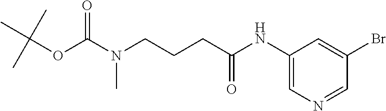

Prepared from Intermediate 60 (210 mg, 0.77 mmol), following the procedure described for Intermediate 51. The title compound (110 mg, 0.29 mmol, 38% yield) was obtained as pale yellow oil.

LC-MS (ESI): m/z (M+1): 372.2 (Method 1).

### Intermediate 62: tert-butyl N-methyl-N-(3-{[5-(4,4,5,5-tetramethyl-1,3,2-dioxaborolan-2-yl)pyridin-3-yl]carbamoyl}propyl)carbamate

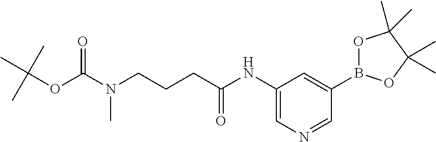

Prepared from Intermediate 61 (110 mg, 0.30 mmol), following similar procedure as for Intermediate 29, affording the title compound (213 mg, recovery assumed quantitative) as brownish solid, that was used in the next step without purification.

LC-MS (ESI): m/z (boronic acid M+1): 338.3 (Method 1).

### Intermediate 63: Tert-Butyl N-[3-({5-[6-(5-chloro-2-fluorophenyl)-2H,3H,4H-pyrido[3,2-b][1,4]oxazin-8-yl]pyridin-3-yl}carbamoyl)propyl]-N-methylcarbamate

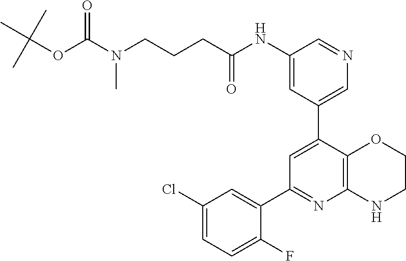

Prepared from Intermediate 24 (87 mg, 0.25 mmol) tert-butyl N-methyl-N-(3-{[5-(4,4,5,5-tetramethyl-1,3,2-dioxaborolan-2-yl)pyridin-3-yl]carbamoyl}propyl)carbamate (Intermediate 62, 0.30 theoretical mmol), following similar procedure as for Intermediate 25, affording the title compound (93 mg, 0.17 mmol, 66% yield) as pale yellow oil.

LC-MS (ESI): m/z (M+1): 556.5 (Method 1).

### Intermediate 64: tert-butyl N-[3-({5-[6-(5-chloro-2-fluorophenyl)-2H,3H,4H-pyrido[3,2-b][1,4]oxazin-8-yl]pyridin-3-yl}formamido)propyl]-N-methylcarbamate

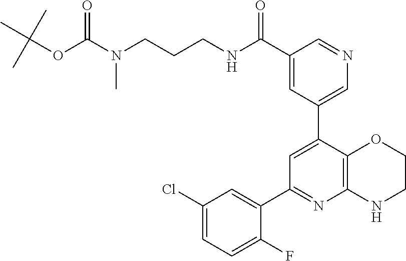

Prepared from Intermediate 32 (100 mg, 0.26 mmol) and tert-butyl N-(3-aminopropyl)-N-methylcarbamate (74.6 μL, 0.39 mmol), following similar procedure as for Intermediate 49, affording the title compound (88 mg, 0.16 mmol, 61% yield) as pale yellow glassy solid.

LC-MS (ESI): m/z (M+1): 556.3 (Method 1).

### Intermediate 65: Tert-Butyl 4-{3-[(5-bromopyridin-3-yl)carbamoyl]propyl}piperazine-1-carboxylate

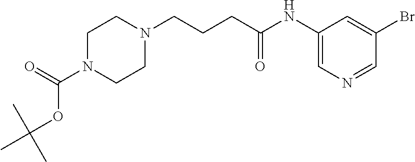

A solution of Intermediate 41 (0.25 g, 0.90 mmol) and tert-butyl piperazine-1-carboxylate (1.6 g, 9.01 mmol) in THE (5.5 mL) was warmed at 70° C. for 72 hrs. Volatiles were removed under vacuum and the crude material was purified by FC on Biotage silica-NH gel (from 10% to 100% of EtOAc in c-Hex, 10% MeOH was used as additive). Proper fraction were collected and purified again by FC on Biotage silica gel (from 10% to 16% of MeOH in DCM) to give the title compound (180 mg, 0.42 mmol, 46% yield) as pale yellow oil that slowly solidifies.

LC-MS (ESI): m/z (M+1): 427.3 (Method 1).

### Intermediate 66: tert-butyl 4-(3-{[5-(4,4,5,5-tetramethyl-1,3,2-dioxaborolan-2-yl)pyridin-3-yl]carbamoyl}propyl)piperazine-1-carboxylate

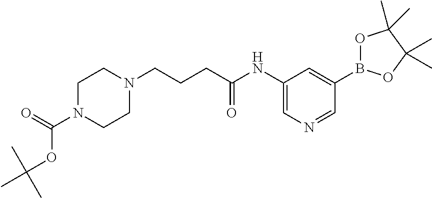

Prepared from Intermediate 65 (126 mg, 0.30 mmol), following similar procedure as for Intermediate 29, affording the title compound (230 mg, recovery assumed quantitative) as brownish solid, that was used in the next step without purification.

LC-MS (ESI): m/z (boronic acid M+1): 393.3 (Method 1).

### Intermediate 67: tert-butyl 4-[3-({5-[6-(5-chloro-2-fluorophenyl)-2H,3H,4H-pyrido[3,2-b][1,4]oxazin-8-yl]pyridin-3-yl}carbamoyl)propyl]piperazine-1-carboxylate

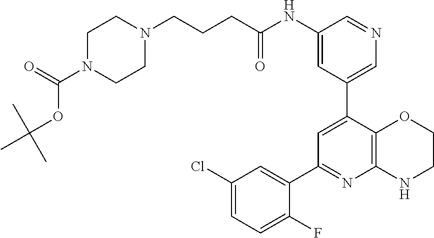

Prepared from Intermediate 24 (67 mg, 0.19 mmol) and tert-butyl 4-(3-{[5-(4,4,5,5-tetramethyl-1,3,2-dioxaborolan-2-yl)pyridin-3-yl]carbamoyl}propyl)piperazine-1-carboxylate (Intermediate 66, 0.30 theoretical mmol), following similar procedure as for Intermediate 25, affording the title compound (86 mg, 0.141 mmol, 72% yield) as pale yellow oil.

LC-MS (ESI): m/z (M+2) 306.4 (Method 1).

### Intermediate 68: 3-bromo-5-[(oxolan-2-yl)methoxy]pyridine

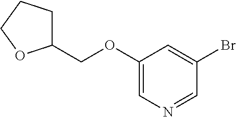 [SMILES: Brc1cncc(OCC2CCCO2)c1]

A mixture of 3-bromo-5-hydroxypyridine (300 mg, 1.72 mmol), K₂CO₃ (477 mg, 3.45 mmol) and 2-(bromomethyl)tetrahydrofuran (0.29 mL, 2.59 mmol) in DMF (11.5 mL) was heated at 80° C. in a PLS, overnight. The reaction mixture was diluted with EtOAc and sat. aqueous NaHCO₃, the phases were separated and the aqueous phase was extracted with EtOAc (2×). The combined organic layers were filtered through a phase separator and concentrated under vacuum. The crude material was dissolved in MeOH and loaded on a SCX column (10 g), washed with MeOH then eluted with 2 N ammonia in MeOH. Basic fractions were evaporated to give the title compound (406 mg, 1.573 mmol, 91% yield) as orange oil.

¹H NMR (400 MHz, Chloroform-d) δ ppm 8.28 (dd, J=6.82, 2.20 Hz, 2H), 7.38-7.45 (m, 1H), 4.23-4.33 (m, 1H), 3.98-4.06 (m, 2H), 3.90-3.97 (m, 1H), 3.81-3.89 (m, 1H), 2.05-2.15 (m, 1H), 1.91-2.03 (m, 2H), 1.70-1.83 (m, 1H).

### Intermediate 69: 3-[(oxolan-2-yl)methoxy]-5-(4,4,5,5-tetramethyl-1,3,2-dioxaborolan-2-yl)pyridine

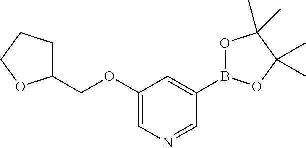 [SMILES: CC1(C)OB(c2cncc(OCC3CCCO3)c2)OC1(C)C]

Prepared from Intermediate 68 (76 mg, 0.30 mmol), following similar procedure as for Intermediate 29, affording the title compound (212 mg, recovery assumed quantitative) as brownish solid, that was used in the next step without purification.

LC-MS (ESI): m/z (boronic acid M+1): 224.2 (Method 1)

### Intermediate 70: 2-amino-4,6-dibromopyridin-3-ol

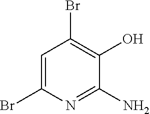

To an ice-cooled solution of 4,6-dibromo-2-nitropyridin-3-ol (5.0 g, 17.8 mmol) and nickel(II) chloride hexahydrate (0.80 g, 16.8 mmol) in a mixture of dry MeOH and dry THE (86 mL, 1:1 ratio), sodium borohydride (2.54 g, 67.1 mmol) was added portionwise. The reaction was stirred at 0° C. for 30 min. The mixture was quenched with cold water and the pH was adjusted to 4-5 using 3 N HCl solution and extracted with EtOAc (2×). The combined organic layers were washed with brine, dried over Na₂SO₄ and evaporated under vacuum, affording 2-amino-4,6-dibromopyridin-3-ol (3.90 g, 14.6 mmol, 87% yield), that was used in the next step without further purification.

LC-MS (ESI): m/z (M+1): 266.9 (Method 1)

### Intermediate 71: 4,6-dibromo-3-(2-chloroethoxy)pyridin-2-amine

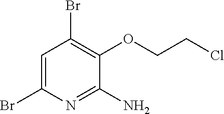 [SMILES: Nc1nc(Br)cc(Br)c1OCCCl]

K₂CO₃ (3.22 g, 23.3 mmol) was added to a stirred solution of Intermediate 70 (3.9 g, 11.7 mmol) in dry DMF (27 mL). The mixture was stirred at RT for 10 min before adding 1-bromo-2-chloroethane (2.51 g, 17.5 mmol). The reaction was stirred at RT for 20 hrs, then it was poured into 5% ice-cold citric acid aqueous solution and extracted with EtOAc. The organic phase was washed with water, dried over Na₂SO₄ and concentrated under vacuum. The residue was purified by FC on Biotage silica gel (20% of EtOAc in c-Hex as eluent), affording the title compound (3.3 g, 9.99 mmol, 86% yield).

LC-MS (ESI): m/z (M+1): 328.9 (Method 1)

### Intermediate 72: 6,8-dibromo-2H,3H,4H-pyrido[3,2-b][1,4]oxazine

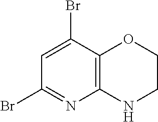 [SMILES: Brc1cc(Br)c2c(n1)NCCO2]

Under N₂ atmosphere, a solution of Intermediate 71 (3.3 g, 9.49 mmol) in dry DMF (50 mL) was added dropwise to an ice-cooled stirred mixture of sodium hydride (60% dispersion in mineral oil, 0.49 g, 12.3 mmol) in dry DMF (15 mL). Once the addition was completed, the reaction was warmed at RT and stirred for 2 hrs, then it was poured into 5% ice-cold citric acid aqueous solution and extracted with EtOAc. The organic phase was washed with water, dried over Na₂SO₄ and concentrated under vacuum, affording 6,8-dibromo-2H,3H,4H-pyrido[3,2-b][1,4]oxazine (3 g, recovery assumed quantitative).

LC-MS (ESI): m/z (M+1): 292.9 (Method 1)

### Intermediate 73: tert-butyl 6,8-dibromo-2H,3H,4H-pyrido[3,2-b][1,4]oxazine-4-carboxylate

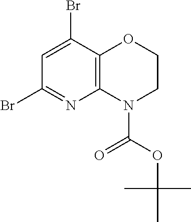 [SMILES: CC(C)(C)OC(=O)N1CCOc2c(Br)cc(Br)nc21]

Under N₂ atmosphere, lithium bis(trimethylsilyl)amide (1.0 M solution in THF, 11.4 mL, 11.4 mmol) was added dropwise to an ice-cooled stirred solution of Intermediate 72 (3.0 g, 9.49 mmol) in dry THE (94 mL). After 5 min, di-tert-butyl dicarbonate (2.49 g, 11.4 mmol) was added. The reaction was allowed to reach the RT and stirred for 1 h. The mixture was poured into an ice-cold NH₄Cl aqueous solution and extracted with EtOAc. Organic layer was separated, dried over Na₂SO₄ and evaporated at reduced pressure, providing the title compound (3.6 g, 9.14 mmol, 96% yield).

LC-MS (ESI): m/z (M+1): 392.9 (Method 2)

### Intermediate 74: Tert-Butyl 8-bromo-6-(6-methylpyridin-2-yl)-2H,3H,4H-pyrido[3,2-b][1,4]oxazine-4-carboxylate

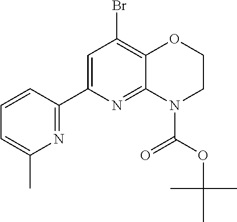 [SMILES: Cc1cccc(-c2cc(Br)c3c(n2)N(C(=O)OC(C)(C)C)CCO3)n1]

In a suitable vial, a solution of Intermediate 73 (500 mg, 1.27 mmol) and 2-methyl-6-(tributylstannyl)pyridine (485 mg, 1.27 mmol) in dry toluene (10 mL) was degassed by N₂ bubbling, before adding Pd(PPh₃)₄ (116 mg, 0.10 mmol). The vial was sealed and heated at 100° C. for 20 hrs. Volatiles were removed under vacuum, the residue was material was suspended in a mixture of Et₂O and 1 N aqueous KF solution and vigorously stirred at RT for 3 hrs. The mixture was filtered through a Celite® pad, then the organic layer was separated, dried over Na₂SO₄ and evaporated at reduced pressure. The residue material was purified by FC on Biotage silica gel (from 0 to 10% of EtOAc in c-Hex as eluent), to give the title compound (166 mg, 0.41 mmol, 32% yield) as white solid.

LC-MS (ESI): m/z (M+1): 406.2 (Method 1)

¹H NMR (500 MHz, DMSO-d₆) δ ppm 8.27 (s, 1H), 8.07 (d, J=7.7 Hz, 1H), 7.83 (t, J=7.8 Hz, 1H), 7.27 (d, J=7.5 Hz, 1H), 4.41 (t, J=4.5 Hz, 2H), 3.93 (t, J=4.5 Hz, 2H), 2.54 (s, 3H), 1.50 (s, 9H).

Note: This procedure gave also the 8-isomer (tert-butyl 6-bromo-8-(6-methylpyridin-2-yl)-2H,3H,4H-pyrido[3,2-][1,4]oxazine-4-carboxylate (241 mg, 0.59 mmol, 47%).

¹H NMR (500 MHz, DMSO-d₆) δ ppm 7.76-7.81 (m, 2H), 7.61 (s, 1H), 7.28-7.34 (m, 1H), 4.33 (t, J=4.5 Hz, 2H), 3.88 (t, J=4.5 Hz, 2H), 2.54 (s, 3H), 1.50 (s, 9H).

### Intermediate 75: tert-butyl 6-(6-methylpyridin-2-yl)-8-{1H-pyrazolo[3,4-b]pyridin-5-yl}-2H,3H,4H-pyrido[3,2-b][1,4]oxazine-4-carboxylate

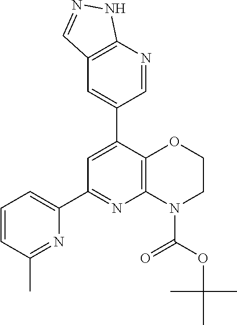 [SMILES: Cc1cccc(-c2cc(-c3cnc4[nH]ncc4c3)c3c(n2)N(C(=O)OC(C)(C)C)CCO3)n1]

Prepared from Intermediate 74 (60 mg, 0.15 mmol) and 5-(4,4,5,5-tetramethyl-1,3,2-dioxaborolan-2-yl)-3H-pyrazolo[3,4-b]pyridine (43.4 mg, 0.18 mmol), following similar procedure as for Intermediate 25, affording the title compound (61 mg, 0.14 mmol, 93% yield) as yellow oil.

LC-MS (ESI): m/z (M+1): 445.4 (Method 1).

### Intermediate 76: 3-bromo-5-(oxolan-3-yloxy)pyridine

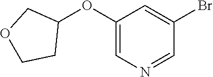 [SMILES: Brc1cncc(OC2CCOC2)c1]

Prepared from 3-bromo-5-hydroxypyridine (300 mg, 1.72 mmol) and 3-bromotetrahydrofuran (0.24 mL, 2.59 mmol), following a similar procedure as for Intermediate 68. The title compound (256 mg, 1.05 mmol, 61% yield) was obtained as colorless oil.

LC-MS (ESI): m/z (M+2): 244.1 (Method 1).

### Intermediate 77: 3-(oxolan-3-yloxy)-5-(4,4,5,5-tetramethyl-1,3,2-dioxaborolan-2-yl)pyridine

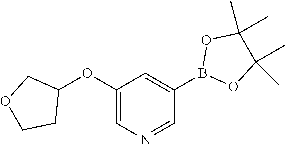 [SMILES: CC1(C)OB(c2cncc(OC3CCOC3)c2)OC1(C)C]

Prepared from Intermediate 76 (72 mg, 0.30 mmol), following similar procedure as for Intermediate 29, affording the title compound (204 mg, recovery assumed quantitative) as brownish solid, that was used in the next step without purification.

LC-MS (ESI): m/z (boronic acid M+1): 210.1 (Method 1).

### Intermediate 78: N-(5-bromopyridin-3-yl)-3-(4-methylpiperazin-1-yl)propenamide

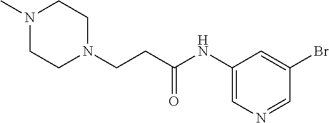

Prepared from Intermediate 27 (300 mg, 1.32 mmol) and 1-methylpiperazine (0.29 mL, 2.64 mmol), following similar procedure as for Intermediate 28, affording the title compound (340 mg, 1.04 mmol, 79% yield) as colorless oil that slowly solidifies.

LC-MS (ESI): m/z (M+1): 327.2 (Method 1).

### Intermediate 79: 3-(4-methylpiperazin-1-yl)-N-[5-(4,4,5,5-tetramethyl-1,3,2-dioxaborolan-2-yl)pyridin-3-yl]propanamide

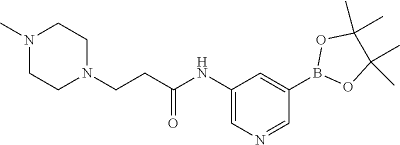

Prepared from Intermediate 78 (85.7 mg, 0.26 mmol), following similar procedure as for Intermediate 29, affording the title compound (207 mg, recovery assumed quantitative) as brownish solid, that was used in the next step without purification.

LC-MS (ESI): m/z (boronic acid M+1): 293.2 (Method 1).

### Intermediate 80: methyl 5-{4-[(tert-butoxy)carbonyl]-6-(6-methylpyridin-2-yl)-2H,3H,4H-pyrido[3,2-b][1,4]oxazin-8-yl}pyridine-3-carboxylate

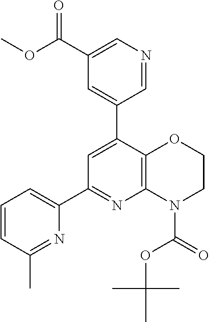 [SMILES: COC(=O)c1cncc(-c2cc(-c3cccc(C)n3)nc3c2OCCN3C(=O)OC(C)(C)C)c1]

Prepared from Intermediate 74 (133 mg, 0.33 mmol) and and 3-(methoxycarbonyl)pyridine-5-boronic acid pinacol ester (103 mg, 0.39 mmol), following similar procedure as for Intermediate 25, affording the title compound (99 mg, 0.21 mmol, 64% yield) as white solid.

LC-MS (ESI): m/z (M+1): 463.4 (Method 1).

### Intermediate 81: Tert-Butyl 8-bromo-6-(5-chloro-2-fluorophenyl)-2H,3H,4H-pyrido[3,2-b][1,4]oxazine-4-carboxylate

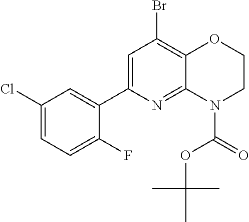 [SMILES: CC(C)(C)OC(=O)N1CCOc2c(Br)cc(-c3cc(Cl)ccc3F)nc21]

To an ice-cooled solution of Intermediate 24 (590 mg, 1.72 mmol) in dry THE (18 mL), lithium bis(trimethylsilyl)amide (1.0 M solution in THF, 2.06 mL, 2.06 mmol) was added and the mixture was stirred for 5 minutes, before adding di-tert-butyl dicarbonate (450 mg, 2.06 mmol). The reaction was stirred at 0 C for 1 h. The mixture was quenched with sat. NH₄Cl aqueous solution and partitioned between EtOAc and brine. The organic phase was separated, filtered through a phase separator, concentrated at reduced pressure and dried at high vacuum overnight to provide the title compound (832 mg, recovery assumed quantitative) as a light brown solid.

LC-MS (ESI): m/z (M+1): 443.2 (Method 1).

### Intermediate 82: 5-(4,4,5,5-tetramethyl-1,3,2-dioxaborolan-2-yl)pyridine-2,3-diamine

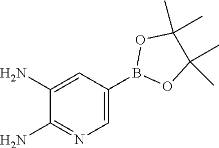 [SMILES: CC1(C)OB(c2cnc(N)c(N)c2)OC1(C)C]

Prepared from 2,3-diamino-5-bromopyridine (100 mg, 0.53 mmol), following similar procedure as for Intermediate 29, affording the title compound (340 mg, recovery assumed quantitative) as brownish solid, that was used in the next step without purification.

LC-MS (ESI): m/z (boronic acid M+1): 154.2 (Method 1).

### Intermediate 83: tert-butyl 6-(5-chloro-2-fluorophenyl)-8-(5,6-diaminopyridin-3-yl)-2H,3H,4H-pyrido[3,2-b][1,4]oxazine-4-carboxylate

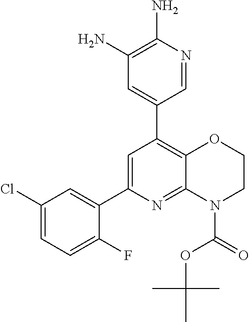 [SMILES: CC(C)(C)OC(=O)N1CCOc2c(-c3cnc(N)c(N)c3)cc(-c3cc(Cl)ccc3F)nc21]

Prepared from Intermediate 81 (120 mg, 0.27 mmol) and Intermediate 82 (0.50 theoretical mmol), following similar procedure as for Intermediate 25, affording the title compound (60 mg, 0.13 mmol, 48% yield) as pale yellow oil.

LC-MS (ESI): m/z (M+1): 472.3 (Method 1).

### Intermediate 84: tert-butyl 6-(5-chloro-2-fluorophenyl)-8-{2-oxo-1H,2H,3H-imidazo[4,5-b]pyridin-6-yl}-2H,3H,4H-pyrido[3,2-b][1,4]oxazine-4-carboxylate

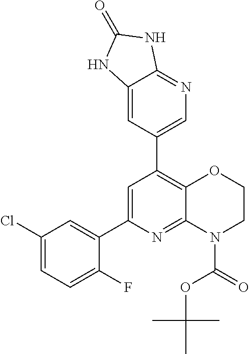 [SMILES: CC(C)(C)OC(=O)N1CCOc2c(-c3cnc4[nH]c(=O)[nH]c4c3)cc(-c3cc(Cl)ccc3F)nc21]

To a solution of Intermediate 83 (60 mg, 0.13 mmol) in THE (1 mL), 1,1′-carbonyldiimidazole (37 mg, 0.23 mmol) was added. The mixture was stirred at 60° C. for 4 hrs. Volatiles were removed under vacuum and the crude material was purified by FC on Biotage silica gel (from 20% to 100% of EtOAc in c-Hex as eluent), affording the title compound (40 mg, 0.08 mmol, 62% yield) as white solid.

LC-MS (ESI): m/z (M+1): 498.3 (Method 1).

### Intermediate 85: 3-bromo-5-[(1-methylpiperidin-4-yl)oxy]pyridine

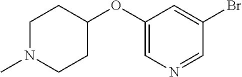

To an ice-cooled suspension 3-bromo-5-hydroxypyridine (300 mg, 1.72 mmol), triphenylphosphine (678 mg, 2.59 mmol), and N-methyl-4-piperidinol (298 mg, 2.59 mmol) in THE (5.8 mL), diisopropyl azodicarboxylate (0.51 mL, 2.59 mmol) was added dropwise and the mixture was immediately heated at 50° C. for 1 h. Volatiles were removed under vaccum. The crude material was dissolved in MeOH and loaded on a SCX column (10 g), washed with MeOH then eluted with 2 N ammonia in MeOH. Basic fractions were evaporated and the residue was further purified by FB on Biotage silica-NH gel (from 0% to 40% of EtOAc in c-Hex as eluent), affording the title compound (305 mg, 1.13 mmol, 65% yield) as colorless oil.

LC-MS (ESI): m/z (M+1): 271.1 (Method 1).

### Intermediate 86: 3-[(1-methylpiperidin-4-yl)oxy]-5-(4,4,5,5-tetramethyl-1,3,2-dioxaborolan-2-yl)pyridine

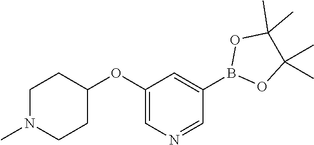

Prepared from Intermediate 85 (75 mg, 0.26 mmol), following similar procedure as for Intermediate 29, affording the title compound (183 mg, recovery assumed quantitative) as brownish solid, that was used in the next step without purification.

LC-MS (ESI): m/z (boronic acid M+1): 237.2 (Method 1).

### Intermediate 87: 3-bromo-5-[(1-methylpiperidin-4-yl)methoxy]pyridine

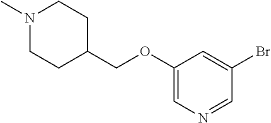 [SMILES: CN1CCC(COc2cncc(Br)c2)CC1]

Prepared from 3-bromo-5-hydroxypyridine (600 mg, 3.44 mmol) and 4-(hydroxymethyl)-1-methylpiperidineN-methyl-4-piperidinol (668 mg, 5.18 mmol), following similar procedure as for Intermediate 85. The title compound (432 mg, 1.52 mmol, 44% yield) was obtained as colorless oil that slowly solidifies.

LC-MS (ESI): m/z (M+1): 285.2 (Method 1).

### Intermediate 88: 3-[(1-methylpiperidin-4-yl)methoxy]-5-(4,4,5,5-tetramethyl-1,3,2-dioxaborolan-2-yl)pyridine

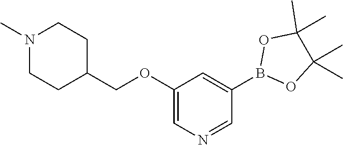

Prepared from Intermediate 87 (75 mg, 0.26 mmol), following similar procedure as for Intermediate 29, affording the title compound (146 mg, recovery assumed quantitative) as brownish solid, that was used in the next step without purification.

LC-MS (ESI): m/z (boronic acid M+1): 251.2 (Method 1).

### Intermediate 89: methyl 4-methyl-5-(4,4,5,5-tetramethyl-1,3,2-dioxaborolan-2-yl)pyridine-3-carboxylate

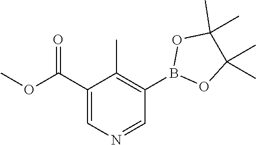

Prepared from methyl 5-bromo-4-methylpyridine-3-carboxylate (62 mg, 0.27 mmol), following similar procedure as for Intermediate 37, affording the title compound (185 mg, recovery assumed quantitative) as brownish solid, that was used in the next step without purification.

LC-MS (ESI): m/z (boronic acid M+1): 196.1 (Method 1).

### Intermediate 90: tert-butyl 8-(5-aminopyridin-3-yl)-6-(5-chloro-2-fluorophenyl)-2H,3H,4H-pyrido[3,2-b][1,4]oxazine-4-carboxylate

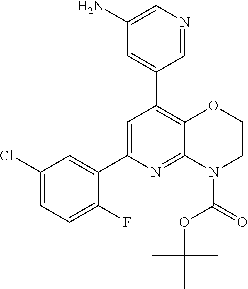 [SMILES: CC(C)(C)OC(=O)N1CCOc2c(-c3cncc(N)c3)cc(-c3cc(Cl)ccc3F)nc21]

Prepared from Intermediate 81 (120 mg, 0.27 mmol) and 5-(4,4,5,5-tetramethyl-1,3,2-dioxaborolan-2-yl)pyridin-3-amine (71.4 mg, 0.32 mmol), following similar procedure as for Intermediate 25, affording the title compound (103 mg, 0.23 mmol, 82% yield) as pale yellow oil, that slowly solidifies.

LC-MS (ESI): m/z (M+1): 457.3 (Method 1).

### Intermediate 91: tert-butyl 6-(5-chloro-2-fluorophenyl)-8-[5-(prop-2-enamido)pyridin-3-yl]-2H,3H,4H-pyrido[3,2-b][1,4]oxazine-4-carboxylate

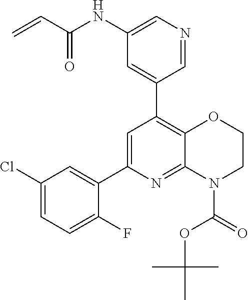 [SMILES: C=CC(=O)Nc1cncc(-c2cc(-c3cc(Cl)ccc3F)nc3c2OCCN3C(=O)OC(C)(C)C)c1]

To an ice-cooled solution of Intermediate 90 (99 mg, 0.22 mmol) in dry DCM (1.5 mL), TEA (91 μL, 0.65 mmol) and acryloyl chloride (22.3 μL, 0.28 mmol) were added. The reaction was stirred for 30 min at 0° C., then it was diluted with DCM and washed with sat. aqueous NaHCO₃ solution (3×) and brine. The organic phase was filtered through a phase separator and dried under vacuum. The crude material was purified by FC on Biotage silica gel (from 30% to 100% of EtOAc in c-Hex as eluent), affording the title compound (49 mg, 0.10 mmol, 45% yield) as orange oil.

LC-MS (ESI): m/z (M+1): 511.3 (Method 1).

### Intermediate 92: Tert-Butyl 8-(5-{3-[bis(2-hydroxyethyl)amino]propanamido}pyridin-3-yl)-6-(5-chloro-2-fluorophenyl)-2H,3H,4H-pyrido[3,2-b][1,4]oxazine-4-carboxylate

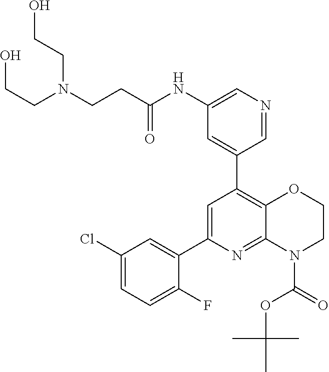 [SMILES: CC(C)(C)OC(=O)N1CCOc2c(-c3cncc(NC(=O)CCN(CCO)CCO)c3)cc(-c3cc(Cl)ccc3F)nc21]

A solution of Intermediate 91 (49 mg, 0.10 mmol) and diethanolamine (0.11 mL, 1.15 mmol) in THE (0.5 mL) was heated at 60° C. for 24 hrs. Volatiles were removed under vacuum. The residue material was taken up with EtOAc and washed with brine (2×), the organic phase was filtered through a phase separator and evaporated under vacuum. The crude was purified by FC on Biotage silica-NH (from 0% to 5% of MeOH in DCM as eluent), affording the title compound (43 mg, 0.07 mmol, 73% yield) as white foam.

LC-MS (ESI): m/z (M-tBu+2): 280.8 (Method 1).

### Intermediate 93: Tert-Butyl 6-(5-chloro-2-fluorophenyl)-8-(5-{3-[(2-hydroxyethyl)(methyl)amino]propanamido}pyridin-3-yl)-2H,3H,4H-pyrido[3,2-b][1,4]oxazine-4-carboxylate

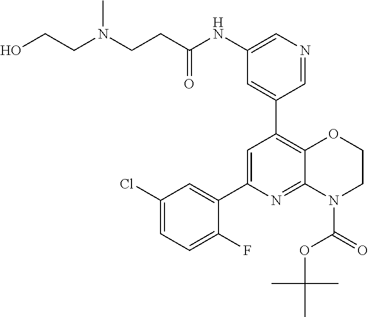 [SMILES: CN(CCO)CCC(=O)Nc1cncc(-c2cc(-c3cc(Cl)ccc3F)nc3c2OCCN3C(=O)OC(C)(C)C)c1]

A solution of Intermediate 91 (70 mg, 0.14 mmol) and 2-(methylamino)ethanol (111 μL, 1.37 mmol) in THE (0.7 mL) was heated at 60° C. for 1 h. Volatiles were removed under vacuum. The residue material was taken up with EtOAc and washed with brine (2×), the organic phase was filtered through a phase separator and evaporated under vacuum. The crude was purified by FC on Biotage silica-NH (from 0% to 3% of MeOH in DCM), affording the title compound (66 mg, 0.11 mmol, 82% yield) as white foam.

LC-MS (ESI): m/z (M-tBu+2): 265.7 (Method 1).

### Intermediate 94: Tert-Butyl 6-(5-chloro-2-fluorophenyl)-8-(5-{3-[methyl(oxetan-3-yl)amino]propanamido}pyridin-3-yl)-2H,3H,4H-pyrido[3,2-b][1,4]oxazine-4-carboxylate

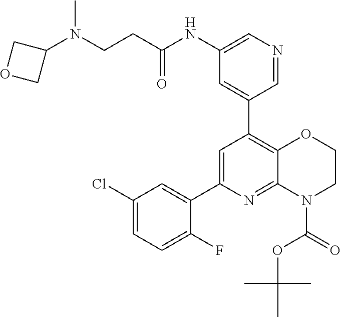 [SMILES: CN(CCC(=O)Nc1cncc(-c2cc(-c3cc(Cl)ccc3F)nc3c2OCCN3C(=O)OC(C)(C)C)c1)C1COC1]

Prepared from Intermediate 91 (70 mg, 0.14 mmol) and N-methyl-3-aminooxetane (122 μL, 1.37 mmol), following the procedure described for Intermediate 92. The title compound (53 mg, 0.09 mmol, 65% yield) was obtained as white foam.

LC-MS (ESI): m/z (M+1): 598.2 (Method 1).

### Intermediate 95: Tert-Butyl 6-(5-chloro-2-fluorophenyl)-8-{5-[3-(4-methyl-3-oxopiperazin-1-yl)propanamido]pyridin-3-yl}-2H,3H,4H-pyrido[3,2-b][1,4]oxazine-4-carboxylate

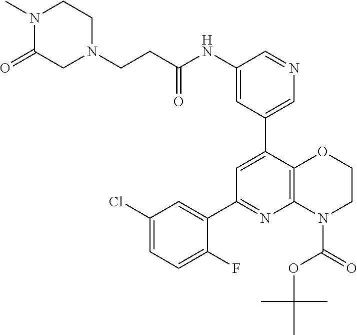 [SMILES: CN1CCN(CCC(=O)Nc2cncc(-c3cc(-c4cc(Cl)ccc4F)nc4c3OCCN4C(=O)OC(C)(C)C)c2)CC1=O]

Prepared from Intermediate 91 (70 mg, 0.14 mmol) and 1-methyl-2-piperazinone (157 mg, 1.37 mmol), following the procedure described for Intermediate 93. The title compound (70 mg, 0.11 mmol, 81% yield) was obtained as white foam.

LC-MS (ESI): m/z (M+1): 625.3 (Method 1).

### Intermediate 96: Tert-Butyl 6-(5-chloro-2-fluorophenyl)-8-{5-[3-(4-methyl-2-oxopiperazin-1-yl)propanamido]pyridin-3-yl}-2H,3H,4H-pyrido[3,2-b][1,4]oxazine-4-carboxylate

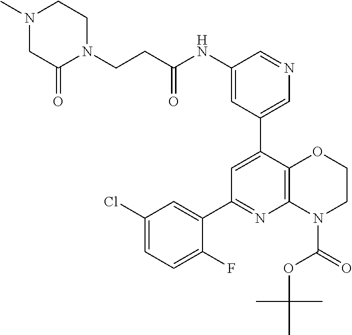 [SMILES: CN1CCN(CCC(=O)Nc2cncc(-c3cc(-c4cc(Cl)ccc4F)nc4c3OCCN4C(=O)OC(C)(C)C)c2)C(=O)C1]

A mixture of Intermediate 91 (70 mg, 0.14 mmol), 4-methylpiperazin-2-one (46.9 mg, 0.41 mmol), and sodium hydroxide (46.6 mg, 1.16 mmol) was supended in THE (1.3 mL)/DMF (0.200 mL) and vigorously stirred at RT for 48 hrs. The reaction was diluted with EtOAc and washed sat. aqueous NaHCO₃ solution (3×) and brine. The organic phase was filtered through a phase separator and concentrated under vacuum. The crude was purified by FC on Biotage silica gel (from 0% to 10% of MeOH in DCM as eluent), affording the title compound (29 mg, 0.05 mmol, 34% yield) as white foam.

LC-MS (ESI): m/z (M+1): 625.3 (Method 2).

### Intermediate 97: Tert-Butyl 6-(5-chloro-2-fluorophenyl)-8-(5-{3-[(2-methoxy-2-oxoethyl)(methyl)amino]propanamido}pyridin-3-yl)-2H,3H,4H-pyrido[3,2-b][1,4]oxazine-4-carboxylate

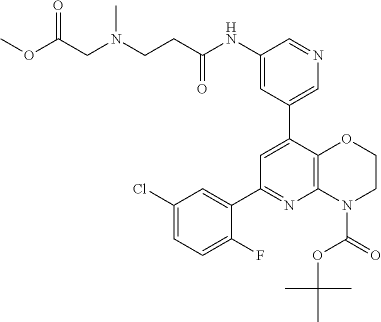 [SMILES: COC(=O)CN(C)CCC(=O)Nc1cncc(-c2cc(-c3cc(Cl)ccc3F)nc3c2OCCN3C(=O)OC(C)(C)C)c1]

To a stirred suspension sarcosine methyl ester hydrochloride (410 mg, 2.94 mmol) and TEA (0.41 mL, 2.94 mmol) in DCM (0.5 mL), Intermediate 91 (50 mg, 0.10 mmol) was added. The reaction was stirred at RT for one week. The mixture was diluted with EtOAc and washed with brine (1×). The organic phase was filtered through a phase separator and concentrated under vacuum. The crude was purified by FC on Biotage silica-NH gel (from 0% to 100% of EtOAc in c-Hex as eluent) affording the title compound (39 mg, 0.06 mmol, 64% yield) as colorless oil, that slowly solidifies.

LC-MS (ESI): m/z (M-tBu+2): 279.7 (Method 1).

### Preparations of Examples

### Example 1

### 3-[6-(5-chloro-2-fluorophenyl)-2H,3H,4H-pyrido[3,2-b][1,4]oxazin-8-yl]pyridine

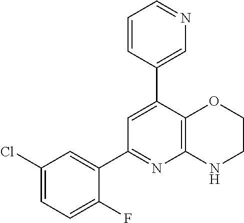 [SMILES: Fc1ccc(Cl)cc1-c1cc(-c2cccnc2)c2c(n1)NCCO2]

To a stirred suspension of Intermediate 6 (0.09 theoretical mmol) and K₂CO₃ (99.5 mg, 0.72 mmol) in dry DMF (1.8 mL), 1,2-dibromoethane (23.3 μL, 0.27 mmol) was added and the mixture was heated at 80° C. for 5 hrs. The reaction was diluted with EtOAc and washed with sat. aqueous NaHCO₃ solution (3×) and brine. The organic phase was filtered through a phase separator and concentrated under vacuum. The crude material was purified by FC on Biotage silica gel (from 30% to 80% of EtOAc in c-Hex), to give the title compound (6 mg, 0.02 mmol, 19% yield) as pale yellow solid.

LC-MS (ESI): m/z (M+1): 342.1 (Method 1)

¹H NMR (500 MHz, DMSO-d₆) δ ppm 8.80 (s, 1H), 8.59 (dd, J=4.7, 1.1 Hz, 1H), 8.01 (br d, J=8.0 Hz, 1H), 7.95 (dd, J=6.7, 2.6 Hz, 1H), 7.49 (dd, J=7.7, 4.9 Hz, 1H), 7.41-7.47 (m, 1H), 7.29-7.38 (m, 1H), 7.23 (br s, 1H), 7.05 (s, 1H), 4.20 (t, J=4.0 Hz, 2H), 3.50 (br s, 2H).

### Example 2

### 4-[6-(5-chloro-2-fluorophenyl)-2H,3H,4H-pyrido[3,2-b][1,4]oxazin-8-yl]isoquinoline

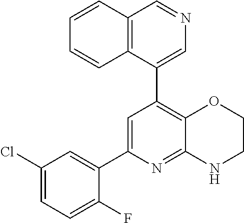 [SMILES: Fc1ccc(Cl)cc1-c1cc(-c2cncc3ccccc23)c2c(n1)NCCO2]

Example 2 was prepared starting from Intermediate 10 (0.29 theoretical mmol), following the procedure described for Example 1. Title compound (17 mg, 0.04 mmol, 15% yield) was obtained as white solid.

LC-MS (ESI): m/z (M+1): 392.1 (Method 1)

¹H NMR (400 MHz, DMSO-d₆) δ ppm 9.37 (s, 1H), 8.46 (s, 1H), 8.21 (d, J=7.9 Hz, 1H), 8.02 (dd, J=6.8, 2.9 Hz, 1H), 7.63-7.87 (m, 3H), 7.44 (dt, J=8.7, 3.5 Hz, 1H), 7.27-7.36 (m, 2H), 7.01 (d, J=1.5 Hz, 1H), 4.09 (br s, 2H), 3.48 (br s, 2H).

### Example 3

### 4-[6-(6-methylpyridin-2-yl)-2H,3H,4H-pyrido[3,2-b][1,4]oxazin-8-yl]isoquinoline

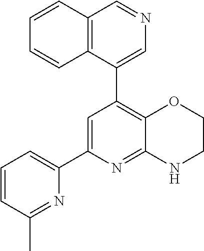 [SMILES: Cc1cccc(-c2cc(-c3cncc4ccccc34)c3c(n2)NCCO3)n1]

Example 3 (40 mg, 0.11 mmol, 74% yield) was prepared starting from Intermediate 15 (50 mg, 0.15 mmol), following the procedure described for Example 1.

LC-MS (ESI): m/z (M+1): 355.1 (Method 2)

¹H NMR (400 MHz, DMSO-d₆) δ ppm 9.37 (s, 1H), 8.46 (s, 1H), 8.22 (d, J 8.0 Hz, 1H), 8.04 (d, J=7.7 Hz, 1H), 7.70-7.82 (m, 3H), 7.66 (d, J=7.7 Hz, 1H), 7.54 (s, 1H), 7.18 (d, J=7.7 Hz, 1H), 7.08-7.13 (m, 1H), 4.08 (t, J=4.0 Hz, 2H), 3.48 (m, J=2.2 Hz, 2H), 2.44 (s, 3H).

### Example 4

### 4-[6-(4-methyl-1,3-thiazol-2-yl)-2H,3H,4H-pyrido[3,2-b][1,4]oxazin-8-yl]isoquinoline

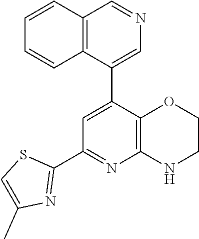 [SMILES: Cc1csc(-c2cc(-c3cncc4ccccc34)c3c(n2)NCCO3)n1]

Example 4 (18 mg, 0.05 mmol, 33% yield) was prepared starting from Intermediate 19 (50 mg, 0.15 mmol), following the procedure described for Example 1.

LC-MS (ESI): m/z (M+1): 361.0 (Method 2)

¹H NMR (500 MHz, DMSO-d₆) δ ppm 9.36-9.39 (m, 1H), 8.47 (s, 1H), 8.20-8.23 (m, 1H), 7.71-7.82 (m, 2H), 7.68 (dd, J=8.2, 0.8 Hz, 1H), 7.34-7.39 (m, 1H), 7.26 (d, J=0.8 Hz, 1H), 7.23 (s, 1H), 4.02-4.17 (m, 2H), 3.40-3.53 (m, 2H), 2.35 (q, J=0.8 Hz, 3H).

### Example 5

### 5-[6-(5-chloro-2-fluorophenyl)-2H,3H,4H-pyrido[3,2-b][1,4]oxazin-8-yl]pyridin-3-amine

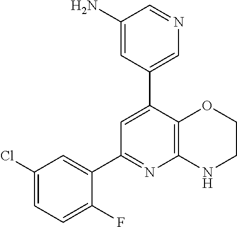 [SMILES: Nc1cncc(-c2cc(-c3cc(Cl)ccc3F)nc3c2OCCN3)c1]

In a suitable vial, a mixture of Intermediate 24 (50 mg, 0.15 mmol), 3-aminopyridine-5-boronic acid pinacol ester (38.4 mg, 0.17 mmol), Na₂CO₃ (30.9 mg, 0.29 mmol) and Pd(dppf)Cl₂ DCM (5.96 mg, 0.01 mmol) dissolved in 1,4-dioxane/water (1.8 mL, 3:1 ratio). The vessel was sealed, evacuated and backfilled with N₂ (3×), then heated at 80° C. for 45 min in a PLS. The reaction was diluted with EtOAc, filtered through a Celite® pad, washing with EtOAc. The filtrate was washed with acidified brine. The organic phase was filtered through a phase separator and evaporated under vacuum. The crude material was purified by FC on Biotage silica-NH gel (from 30% to 100% of EtOAc in c-Hex as eluent), affording the title compound (47 mg, 0.13 mmol, 91% yield) as an ivory solid.

LC-MS (ESI): m/z (M+1): 357.1 (Method 1)

¹H NMR (500 MHz, DMSO-d₆) δ ppm 7.95 (dd, J=6.9, 2.7 Hz, 1H), 7.91-7.93 (m, 2H), 7.43 (ddd, J=8.5, 3.8, 2.7 Hz, 1H), 7.33 (dd, J=11.0, 8.8 Hz, 1H), 7.17 (s, 1H), 7.11 (t, J=2.2 Hz, 1H), 6.98 (d, J=1.6 Hz, 1H), 5.41 (s, 2H), 4.18 (t, J=4.3 Hz, 2H), 3.44-3.51 (m, 2H).

### Example 6

### 5-[6-(5-chloro-2-fluorophenyl)-2H,3H,4H-pyrido[3,2-b][1,4]oxazin-8-yl]pyridine-3-carboxamide

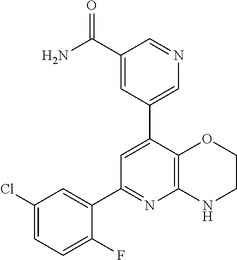 [SMILES: NC(=O)c1cncc(-c2cc(-c3cc(Cl)ccc3F)nc3c2OCCN3)c1]

To a stirred suspension of Intermediate 25 (40 mg, 0.11 mmol) in a DMSO/Ethanol mixture (2 mL, 1:1 ratio), 1 N NaOH aqueous solution (109 μL, 0.11 mmol) and hydrogen peroxide solution (30% w/w in water, 33.4 μL, 0.33 mmol) were subsequently added. The reaction was stirred at RT for 30 min. The mixture was diluted with water and extracted with EtOAc (3×). The combined organic layers were washed with brine, then filtered through a phase separator and concentrated under vacuum. The residue was purified by FC on Biotage Silica-NH gel (from 30% to 100% of EtOAc in c-Hex as eluent), affording the title compound (17 mg, 0.04 mmol, 41% yield) as pale yellow solid.

LC-MS (ESI): m/z (M+1): 385.1 (Method 1)

¹H NMR (500 MHz, DMSO-d₆) δ ppm 9.02 (d, J=1.9 Hz, 1H), 8.93 (d, J=1.9 Hz, 1H), 8.38 (t, J=2.1 Hz, 1H), 8.23 (s, 1H), 7.94 (dd, J=6.7, 2.9 Hz, 1H), 7.67 (s, 1H), 7.45 (dt, J=8.3, 3.5 Hz, 1H), 7.34 (dd, J=11.0, 8.8 Hz, 1H), 7.27 (s, 1H), 7.10 (d, J=0.8 Hz, 1H), 4.21 (t, J=4.3 Hz, 2H), 3.46-3.54 (m, 2H).

### Example 7

### 5-[6-(5-chloro-2-fluorophenyl)-2H,3H,4H-pyrido[3,2-b][1,4]oxazin-8-yl]-N-methylpyridine-3-carboxamide

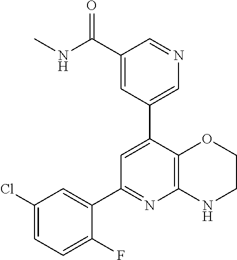 [SMILES: CNC(=O)c1cncc(-c2cc(-c3cc(Cl)ccc3F)nc3c2OCCN3)c1]

Intermediate 26 (52 mg, 0.13 mmol) was suspended in methylamine 2 M solution in MeOH (2.6 mL, 5.2 mmol) and heated at 60° C. for 48 hrs. Volatiles were removed under vacuum. The residue was taken-up and triturated with Et₂O affording the title compound (31 mg, 0.08 mmol, 60% yield) as pale yellow solid.

LC-MS (ESI): m/z (M+1): 399.2 (Method 1)

¹H NMR (400 MHz, DMSO-d₆) δ ppm 8.98 (d, J 2.1 Hz, 1H), 8.93 (d, J 2.1 Hz, 1H), 8.70 (br q, J=4.3 Hz, 1H), 8.34 (t, J=2.1 Hz, 1H), 7.94 (dd, J=6.8, 2.9 Hz, 1H), 7.42-7.49 (m, 1H), 7.34 (dd, J=11.0, 8.8 Hz, 1H), 7.27 (t, J=2.1 Hz, 1H), 7.10 (d, J=1.5 Hz, 1H), 4.21 (t, J=4.3 Hz, 2H), 3.48-3.54 (m, 2H), 2.82 (d, J=4.5 Hz, 3H).

### Example 8

### N-{5-[6-(5-chloro-2-fluorophenyl)-2H,3H,4H-pyrido[3,2-b][1,4]oxazin-8-yl]pyridin-3-yl}-3-(dimethylamino)propanamide

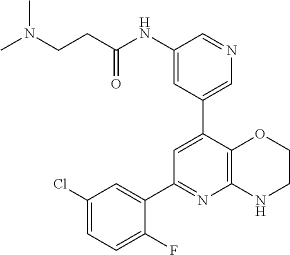 [SMILES: CN(C)CCC(=O)Nc1cncc(-c2cc(-c3cc(Cl)ccc3F)nc3c2OCCN3)c1]

Example 8 was prepared starting from Intermediate 24 (40 mg, 0.12 mmol) and Intermediate 29 (0.18 theoretical mmol) following the procedure described for Example 5. Title compound (30 mg, 0.07 mmol, 57% yield) was obtained as white solid.

LC-MS (ESI): m/z (M+2): 228.7 (Method 1)

¹H NMR (500 MHz, DMSO-d₆) δ ppm 10.34 (s, 1H), 8.77 (d, J=2.5 Hz, 1H), 8.47 (d, J=1.9 Hz, 1H), 8.23 (t, J=2.2 Hz, 1H), 7.95 (dd, J=6.9, 2.7 Hz, 1H), 7.42-7.47 (m, 1H), 7.34 (dd, J=11.0, 8.8 Hz, 1H), 7.25 (s, 1H), 7.03 (d, J=1.4 Hz, 1H), 4.20 (t, J=4.4 Hz, 2H), 3.48-3.54 (m, 2H), 2.55-2.60 (m, 2H), 2.48 (d, J=1.1 Hz, 2H), 2.17 (s, 6H).

### Example 9

### N-{5-[6-(5-chloro-2-fluorophenyl)-2H,3H,4H-pyrido[3,2-b][1,4]oxazin-8-yl]pyridin-3-yl}-3-(morpholin-4-yl)propenamide

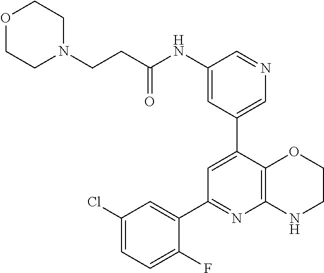 [SMILES: O=C(CCN1CCOCC1)Nc1cncc(-c2cc(-c3cc(Cl)ccc3F)nc3c2OCCN3)c1]

Example 9 was prepared starting from Intermediate 24 (40 mg, 0.12 mmol) and 3-(morpholin-4-yl)-N-[5-(4,4,5,5-tetramethyl-1,3,2-dioxaborolan-2-yl)pyridin-3-yl]propanamide (Intermediate 31, 0.18 theoretical mmol) following the procedure described for Example 5. Title compound (44 mg, 0.09 mmol, 76% yield) was obtained as white solid.

LC-MS (ESI): m/z (M+2): 249.8 (Method 1)

¹H NMR (500 MHz, DMSO-d₆) δ ppm 10.35 (s, 1H), 8.77 (d, J=2.5 Hz, 1H), 8.48 (d, J=1.9 Hz, 1H), 8.22 (t, J=2.2 Hz, 1H), 7.95 (dd, J=6.7, 2.9 Hz, 1H), 7.42-7.48 (m, 1H), 7.34 (dd, J=11.0, 8.8 Hz, 1H), 7.25 (br s, 1H), 7.03 (d, J=1.6 Hz, 1H), 4.20 (t, J=4.4 Hz, 2H), 3.57 (t, J=4.5 Hz, 4H), 3.48-3.53 (m, 2H), 2.60-2.69 (m, 2H), 2.51-2.57 (m, 2H), 2.41 (br s, 4H).

### Example 10

### 6-(5-chloro-2-fluorophenyl)-8-{1H-pyrazolo[3,4-b]pyridin-5-yl}-2H,3H,4H-pyrido[3,2-b][1,4]oxazine

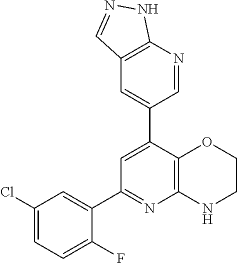 [SMILES: Fc1ccc(Cl)cc1-c1cc(-c2cnc3[nH]ncc3c2)c2c(n1)NCCO2]

Example 10 was prepared starting from Intermediate 24 (40 mg, 0.12 mmol) and 5-(4,4,5,5-tetramethyl-1,3,2-dioxaborolan-2-yl)-1H-pyrazolo[3,4-b]pyridine (34.2 mg, 0.14 mmol), following the procedure described for Example 5. Title compound (19 mg, 0.05 mmol, 43% yield) was obtained as white solid.

LC-MS (ESI): m/z (M+1): 382.1 (Method 1)

¹H NMR (400 MHz, DMSO-d₆) δ ppm 13.76 (br s, 1H), 8.74 (d, J=2.0 Hz, 1H), 8.44 (d, J=2.0 Hz, 1H), 8.21 (s, 1H), 7.96 (dd, J=6.8, 2.9 Hz, 1H), 7.41-7.49 (m, 1H), 7.34 (dd, J=11.0, 8.8 Hz, 1H), 7.19 (s, 1H), 7.12 (d, J=1.8 Hz, 1H), 4.21 (t, J=4.3 Hz, 2H), 3.51 (br s, 2H).

### Example 11

### 5-[6-(5-chloro-2-fluorophenyl)-2H,3H,4H-pyrido[3,2-b][1,4]oxazin-8-yl]-N-[2-(morpholin-4-yl)ethyl]pyridine-3-carboxamide

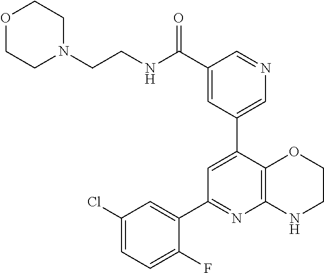 [SMILES: O=C(NCCN1CCOCC1)c1cncc(-c2cc(-c3cc(Cl)ccc3F)nc3c2OCCN3)c1]

A mixture of Intermediate 32 (60 mg, 0.16 mmol) and HATU (71 mg, 0.19 mmol) was suspended in dry DMF (3.2 mL). DIPEA (81 μL, 0.47 mmol) was added and the mixture stirred at RT for 15 min, before adding 2-(morpholin-4-yl)ethan-1-amine (30.0 μL, 0.23 mmol). The reaction was stirred at RT for 1 h, then was diluted with EtOAc and washed with sat. aqueous NaHCO₃ solution (3×) and brine, filtered through a phase separator and concentrated under vacuum. The crude material was purified by FC on Biotage silica-NH gel (from 20% to 100% of EtOAc in c-Hex, 10% MeOH was used as additive), affording the title compound (48 mg, 0.10 mmol, 62% yield) as pale yellow solid.

LC-MS (ESI): m/z (M+2): 249.7 (Method 1)

¹H NMR (400 MHz, DMSO-d₆) δ ppm 8.98 (d, J=2.2 Hz, 1H), 8.93 (d, J=2.2 Hz, 1H), 8.68 (t, J=5.7 Hz, 1H), 8.34 (t, J=2.1 Hz, 1H), 7.94 (dd, J=6.8, 2.9 Hz, 1H), 7.45 (ddd, J=8.8, 4.2, 2.8 Hz, 1H), 7.34 (dd, J=11.0, 8.8 Hz, 1H), 7.25-7.29 (m, 1H), 7.10 (d, J=1.5 Hz, 1H), 4.21 (t, J=4.3 Hz, 2H), 3.54-3.60 (m, 4H), 3.48-3.54 (m, 2H), 3.42 (q, J=6.6 Hz, 2H), 2.46-2.49 (m, 2H), 2.38-2.46 (m, 4H).

### Example 12

### N-{5-[6-(5-chloro-2-fluorophenyl)-2H,3H,4H-pyrido[3,2-b][1,4]oxazin-8-yl]pyridin-3-yl}-3-(pyrrolidin-1-yl)propanamide

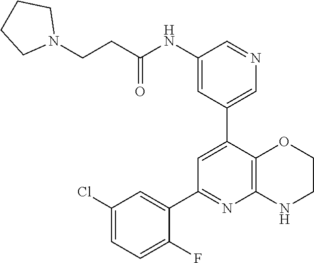 [SMILES: O=C(CCN1CCCC1)Nc1cncc(-c2cc(-c3cc(Cl)ccc3F)nc3c2OCCN3)c1]

Example 12 was prepared starting from Intermediate 24 (40 mg, 0.12 mmol) and Intermediate 34 (0.18 theoretical mmol), following the procedure described for Example 5. Title compound (33 mg, 0.07 mmol, 59% yield) was obtained as white solid.

LC-MS (ESI): m/z (M+2): 241.8 (Method 1)

¹H NMR (400 MHz, DMSO-d₆) δ ppm 10.38 (s, 1H), 8.77 (d, J=2.4 Hz, 1H), 8.47 (d, J=1.8 Hz, 1H), 8.23 (t, J=2.2 Hz, 1H), 7.96 (dd, J=6.8, 2.9 Hz, 1H), 7.41-7.48 (m, 1H), 7.34 (dd, J=11.0, 8.8 Hz, 1H), 7.25 (s, 1H), 7.03 (d, J=1.5 Hz, 1H), 4.20 (t, J=4.3 Hz, 2H), 3.46-3.54 (m, 2H), 2.69-2.76 (m, 2H), 2.39-2.54 (m, 6H), 1.68 (dt, J=6.6, 3.1 Hz, 4H).

### Example 13

### 6-(5-chloro-2-fluorophenyl)-8-{1H-pyrrolo[2,3-b]pyridin-5-yl}-2H,3H,4H-pyrido[3,2-b][1,4]oxazine

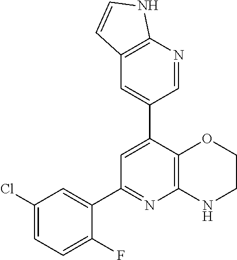 [SMILES: Fc1ccc(Cl)cc1-c1cc(-c2cnc3[nH]ccc3c2)c2c(n1)NCCO2]

Example 13 was prepared starting from Intermediate 24 (50 mg, 0.15 mmol) and 5-(4,4,5,5-tetramethyl-[1,3,2]dioxaborolan-2-yl)-1H-pyrrolo[2,3-b]pyridine (42.6 mg, 0.17 mmol), following the procedure described for Example 5. Title compound (36 mg, 0.10 mmol, 65% yield) was obtained as white solid.

LC-MS (ESI): m/z (M+2): 191.2 (Method 1)

¹H NMR (400 MHz, DMSO-d₆) δ ppm 11.75 (br s, 1H), 8.43 (d, J=2.0 Hz, 1H), 8.17 (d, J=2.0 Hz, 1H), 7.97 (dd, J=6.7, 2.7 Hz, 1H), 7.52 (t, J=2.9 Hz, 1H), 7.44 (dt, J=8.3, 3.6 Hz, 1H), 7.33 (dd, J=11.1, 8.9 Hz, 1H), 7.08-7.18 (m, 2H), 6.51 (dd, J=3.2, 1.4 Hz, 1H), 4.20 (t, J=4.2 Hz, 2H), 3.51 (br s, 2H).

### Example 14

### 6-(5-chloro-2-fluorophenyl)-8-{3H-imidazo[4,5-b]pyridin-6-yl}-2H,3H,4H-pyrido[3,2-b][1,4]oxazine

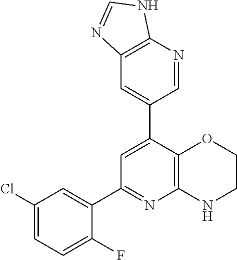 [SMILES: Fc1ccc(Cl)cc1-c1cc(-c2cnc3[nH]cnc3c2)c2c(n1)NCCO2]

Example 14 was prepared starting from Intermediate 24 (50 mg, 0.15 mmol) and 6-(4,4,5,5-tetramethyl-1,3,2-dioxaborolan-2-yl)-3H-imidazo[4,5-b]pyridine (43 mg, 0.17 mmol), following the procedure described for Example 5. Title compound (30 mg, 0.08 mmol, 54% yield) was obtained as white solid.

LC-MS (ESI): m/z (M+2): 191.6 (Method 1)

¹H NMR (500 MHz, DMSO-d₆) δ ppm 13.00 (br s, 1H), 8.57 (s, 1H), 8.49 (s, 1H), 8.21 (br s, 1H), 7.97 (dd, J=6.9, 2.7 Hz, 1H), 7.44 (dt, J=8.5, 3.4 Hz, 1H), 7.34 (dd, J=10.8, 8.9 Hz, 1H), 7.18 (s, 1H), 7.12 (d, J=1.4 Hz, 1H), 4.21 (t, J=4.3 Hz, 2H), 3.51 (br s, 2H).

### Example 15

### N-{5-[6-(5-chloro-2-fluorophenyl)-2H,3H,4H-pyrido[3,2-b][1,4]oxazin-8-yl]pyridin-3-yl}-2-(dimethylamino)acetamide

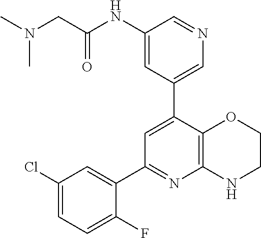 [SMILES: CN(C)CC(=O)Nc1cncc(-c2cc(-c3cc(Cl)ccc3F)nc3c2OCCN3)c1]

Example 15 was prepared starting from Intermediate 24 (40 mg, 0.12 mmol) and Intermediate 37 (0.18 theoretical mmol), following the procedure described for Example 5. Title compound (26 mg, 0.06 mmol, 51% yield) was obtained as white solid.

LC-MS (ESI): m/z (M+1): 442.2 (Method 2).

¹H NMR (400 MHz, DMSO-d₆) δ ppm 10.07 (s, 1H), 8.88 (d, J=2.4 Hz, 1H), 8.50 (d, J=2.0 Hz, 1H), 8.29 (t, J=2.1 Hz, 1H), 7.95 (dd, J=6.8, 2.9 Hz, 1H), 7.40-7.50 (m, 1H), 7.34 (dd, J=11.0, 8.8 Hz, 1H), 7.24 (s, 1H), 7.03 (d, J=1.5 Hz, 1H), 4.20 (t, J=4.2 Hz, 2H), 3.41-3.58 (m, 2H), 3.12 (s, 2H), 2.29 (s, 6H).

### Example 16

### N-{5-[6-(5-chloro-2-fluorophenyl)-2H,3H,4H-pyrido[3,2-b][1,4]oxazin-8-yl]pyridin-3-yl}-3-(piperazin-1-yl)propanamide

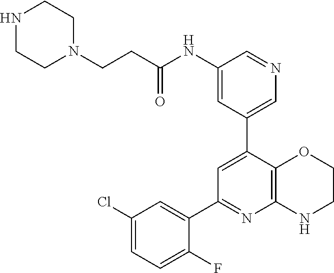 [SMILES: O=C(CCN1CCNCC1)Nc1cncc(-c2cc(-c3cc(Cl)ccc3F)nc3c2OCCN3)c1]

Intermediate 40 (58 mg, 0.1 mmol) was dissolved in DCM/TFA (1.9 mL, 4:1 ratio) and the reaction was stirred at RT for 1 h. Toluene (2 mL) was added and the volatiles were removed under vacuum. The crude material was charged on SCX (2 g, washing with MeOH, eluting with 1 N ammonia in MeOH). Basic fractions were evaporated then purified by reverse FC on Biotage C18 cartridge (from 5% to 45% of MeCN in water 0.1% NH₄OH as eluent). Evaporation of opportune fractions provided title compound (27 mg, 0.05 mmol, 56% yield).

LC-MS (ESI): m/z (M+1): 497.2 (Method 2)

¹H NMR (500 MHz, DMSO-d₆) δ ppm 10.46 (s, 1H), 8.77 (d, J=2.2 Hz, 1H), 8.47 (d, J=1.9 Hz, 1H), 8.22 (t, J=2.2 Hz, 1H), 7.95 (dd, J=6.7, 2.9 Hz, 1H), 7.42-7.50 (m, 1H), 7.34 (dd, J=11.0, 8.8 Hz, 1H), 7.25 (s, 1H), 7.03 (d, J=1.6 Hz, 1H), 4.20 (t, J=4.4 Hz, 2H), 3.46-3.54 (m, 2H), 2.68 (t, J=4.8 Hz, 4H), 2.56-2.63 (m, 2H), 2.47-2.54 (m, 2H), 2.35-2.46 (m, 1H), 2.34 (br s, 4H).

### Example 17

### N-{5-[6-(5-chloro-2-fluorophenyl)-2H,3H,4H-pyrido[3,2-b][1,4]oxazin-8-yl]pyridin-3-yl}-4-(dimethylamino)butanamide

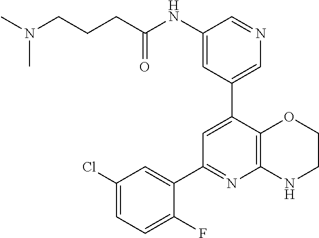 [SMILES: CN(C)CCCC(=O)Nc1cncc(-c2cc(-c3cc(Cl)ccc3F)nc3c2OCCN3)c1]

In a suitable vial, a mixture of Intermediate 46 (50 mg, 0.17 mmol) and Intermediate 43 (0.25 theoretical mmol), Na₂CO₃ (35.4 mg, 0.33 mmol) and PdCl₂(dtbpf) (16 mg, 0.03 mmol) was suspended in 1,4-dioxane/water (2.1 mL, 3:1 ratio). The mixture was evacuated and backfilled with N₂ (3×), then heated at 80° C. in a PLS overnight. The reaction was diluted with EtOAc, filtered through a Celite® pad, washing with EtOAc. The filtrate was washed with acidified brine. The organic phase was filtered through a phase separator and evaporated under vacuum. The crude material was purified by FC on Biotage silica-NH gel (from 30% to 100% of EtOAc in c-Hex, then DCM/MeOH from 0 to 4%), then by reverse FC on Biotage C18 cartridge (from 5% to 20% of MeCN+0.1% HCOOH in water+0.1% HCOOH as eluent). The formate salt was purified again by reverse FC on Biotage C18 cartridge (from 5% to 75% of MeCN in water+0.1% NH₄OH), obtaining the title compound (12 mg, 0.026 mmol, 15% yield) as white solid.

LC-MS (ESI): m/z (M+1): 470.2 (Method 2)

¹H NMR (400 MHz, DMSO-d₆) δ ppm 10.24 (s, 1H), 8.78 (d, J=2.4 Hz, 1H), 8.46 (d, J=2.0 Hz, 1H), 8.22 (t, J=2.0 Hz, 1H), 7.96 (dd, J=6.8, 2.6 Hz, 1H), 7.39-7.51 (m, 1H), 7.34 (dd, J=11.1, 8.9 Hz, 1H), 7.25 (s, 1H), 7.03 (d, J=1.5 Hz, 1H), 4.20 (t, J=4.2 Hz, 2H), 3.50 (br d, J=2.4 Hz, 2H), 2.38 (t, J=7.5 Hz, 2H), 2.23 (t, J=7.0 Hz, 2H), 2.12 (s, 6H), 1.73 (quin, J=7.2 Hz, 2H).

### Example 18

### N-{5-[6-(5-chloro-2-fluorophenyl)-2H,3H,4H-pyrido[3,2-b][1,4]oxazin-8-yl]pyridin-3-yl}-3-(piperidin-1-yl)propanamide

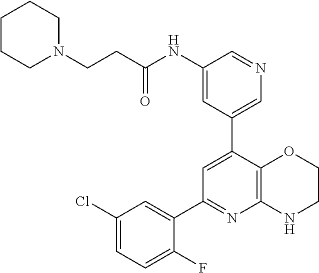

Example 18 was prepared starting from Intermediate 46 (40 mg, 0.13 mmol) and Intermediate 48 (0.19 theoretical mmol), following the procedure described for Example 5. Title compound (20.9 mg, 0.04 mmol, 31% yield) was obtained as white solid.

LC-MS (ESI): m/z (M+1): 496.2 (Method 2)

¹H NMR (400 MHz, DMSO-d₆) δ ppm 10.48 (br s, 1H), 8.77 (d, J 2.2 Hz, 1H), 8.47 (d, J=2.0 Hz, 1H), 8.21-8.24 (m, 1H), 7.95 (dd, J=6.8, 2.6 Hz, 1H), 7.40-7.50 (m, 1H), 7.34 (dd, J=11.0, 8.8 Hz, 1H), 7.25 (s, 1H), 7.03 (d, J=1.3 Hz, 1H), 4.20 (t, J=4.2 Hz, 2H), 3.50 (br d, J=2.4 Hz, 2H), 2.57-2.65 (m, 2H), 2.50-2.53 (m, 2H), 2.38 (br s, 4H), 1.32-1.58 (m, 6H).

### Example 19

### Methyl 5-[6-(5-chloro-2-fluorophenyl)-2H,3H,4H-pyrido[3,2-b][1,4]oxazin-8-yl]pyridine-3-carboxylate

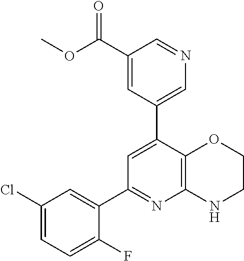 [SMILES: COC(=O)c1cncc(-c2cc(-c3cc(Cl)ccc3F)nc3c2OCCN3)c1]

Title compound was also described as Intermediate 26. Therefore, for detailed information about the synthesis of Example 19, see preparation of Intermediate 26.

¹H NMR (400 MHz, DMSO-d₆) δ ppm 9.09 (d, J 2.0 Hz, 1H), 9.04 (d, J 2.0 Hz, 1H), 8.46 (t, J=2.1 Hz, 1H), 7.95 (dd, J=6.8, 2.9 Hz, 1H), 7.42-7.49 (m, 1H), 7.34 (dd, J=11.0, 8.8 Hz, 1H), 7.29 (s, 1H), 7.10 (d, J=1.3 Hz, 1H), 4.22 (t, J=4.2 Hz, 2H), 3.92 (s, 3H), 3.51 (br d, J=2.4 Hz, 2H).

### Example 20

### 5-[6-(5-chloro-2-fluorophenyl)-2H,3H,4H-pyrido[3,2-b][1,4]oxazin-8-yl]-N-[2-(piperazin-1-yl)ethyl]pyridine-3-carboxamide

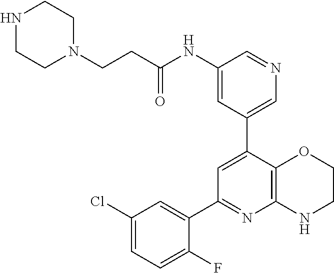

Prepared from Intermediate 49 (53 mg, 0.09 mmol), following the procedure described for Example 16. Title compound (28 mg, 0.06 mmol, 63% yield) was obtained as pale yellow solid.

LC-MS (ESI): m/z (M+1): 497.3 (Method 2)

¹H NMR (400 MHz, DMSO-d₆) δ ppm 8.97 (d, J=2.2 Hz, 1H), 8.92 (d, J=2.2 Hz, 1H), 8.66 (t, J=5.6 Hz, 1H), 8.33 (t, J=2.1 Hz, 1H), 7.94 (dd, J=6.7, 2.7 Hz, 1H), 7.41-7.53 (m, 1H), 7.34 (dd, J=10.9, 8.9 Hz, 1H), 7.27 (s, 1H), 7.10 (d, J=1.5 Hz, 1H), 4.21 (t, J=4.2 Hz, 2H), 3.46-3.59 (m, 2H), 3.40 (q, J=6.6 Hz, 2H), 2.67 (t, J=4.7 Hz, 4H), 2.44 (t, J=7.0 Hz, 2H), 2.34 (br s, 4H), 2.01 (br s, 1H).

### Example 21

### 5-[6-(5-chloro-2-fluorophenyl)-2H,3H,4H-pyrido[3,2-b][1,4]oxazin-8-yl]-N-[3-(dimethylamino)propyl]pyridine-3-carboxamide

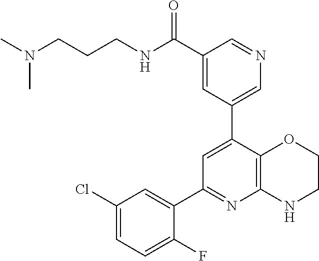

Prepared from Intermediate 32 (50 mg, 0.13 mmol) and 3-(dimethylamino)-1-propylamine (20 μL, 0.19 mmol), following similar procedure as for Example 11. The title compound (35 mg, 0.07 mmol, 57% yield) was obtained as pale yellow solid.

LC-MS (ESI): m/z (M+1): 470.2 (Method 2)

¹H NMR (400 MHz, DMSO-d₆) δ ppm 8.98 (d, J=2.0 Hz, 1H), 8.92 (d, J=2.0 Hz, 1H), 8.74 (t, J=5.5 Hz, 1H), 8.33 (t, J=2.1 Hz, 1H), 7.94 (dd, J=6.8, 2.6 Hz, 1H), 7.42-7.49 (m, 1H), 7.34 (dd, J=11.0, 8.8 Hz, 1H), 7.26 (s, 1H), 7.10 (d, J=1.8 Hz, 1H), 4.21 (t, J=4.3 Hz, 2H), 3.47-3.56 (m, 2H), 3.28-3.36 (m, 2H), 2.26 (t, J=7.2 Hz, 2H), 2.13 (s, 6H), 1.67 (t, J=7.2 Hz, 2H).

### Example 22

### N-{5-[6-(5-chloro-2-fluorophenyl)-2H,3H,4H-pyrido[3,2-b][1,4]oxazin-8-yl]pyridin-3-yl}-3-(methylamino)propanamide

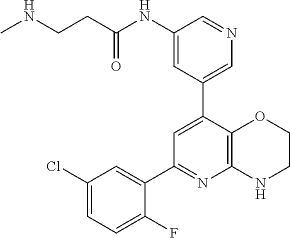

Prepared from Intermediate 53 (80 mg, 0.15 mmol), following the procedure described for Example 16. Title compound (51.2 mg, 0.116 mmol, 78.5% yield) was obtained as pale yellow solid.

LC-MS (ESI): m/z (M+1): 442.2 (Method 2)

¹H NMR (400 MHz, DMSO-d₆) δ ppm 10.38 (br s, 1H), 8.77 (d, J=2.4 Hz, 1H), 8.47 (d, J=2.0 Hz, 1H), 8.23 (t, J=2.1 Hz, 1H), 7.96 (dd, J=6.8, 2.9 Hz, 1H), 7.41-7.49 (m, 1H), 7.34 (dd, J=11.2, 8.8 Hz, 1H), 7.24 (s, 1H), 7.03 (d, J=1.5 Hz, 1H), 4.20 (t, J=4.4 Hz, 2H), 3.50 (br d, J=1.8 Hz, 2H), 2.72-2.83 (m, 2H), 2.44-2.51 (m, 2H), 2.29 (s, 3H), 1.82 (br s, 1H).

### Example 23

### 5-[6-(5-chloro-2-fluorophenyl)-2H,3H,4H-pyrido[3,2-b][1,4]oxazin-8-yl]-N-[2-(methylamino)ethyl]pyridine-3-carboxamide

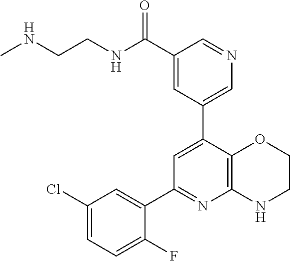

Prepared from Intermediate 54 (50 mg, 0.09 mmol), following the procedure described for Example 16. Title compound (31.8 mg, 0.07 mmol, 78% yield) was obtained as pale yellow solid.

LC-MS (ESI): m/z (M+1): 442.2 (Method 2)

¹H NMR (400 MHz, DMSO-d₆) δ ppm 8.99 (d, J=2.0 Hz, 1H), 8.92 (d, J=2.2 Hz, 1H), 8.65 (br t, J=5.5 Hz, 1H), 8.35 (t, J=2.1 Hz, 1H), 7.94 (dd, J=6.8, 2.6 Hz, 1H), 7.42-7.49 (m, 1H), 7.34 (dd, J=11.0, 8.8 Hz, 1H), 7.24-7.28 (m, 1H), 7.10 (d, J=1.8 Hz, 1H), 4.21 (t, J=4.4 Hz, 2H), 3.48-3.56 (m, 2H), 3.37 (q, J=6.4 Hz, 2H), 2.62-2.69 (m, 2H), 2.29 (s, 3H), 1.68-2.15 (m, 1H).

### Example 24

### 2-(dimethylamino)ethyl 5-[6-(5-chloro-2-fluorophenyl)-2H,3H,4H-pyrido[3,2-b][1,4]oxazin-8-yl]pyridine-3-carboxylate

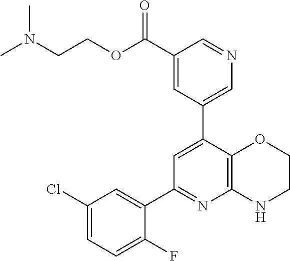

Intermediate 32 (50 mg, 0.13 mmol) and HATU were suspended in dry DMF (2.6 mL), before adding DIPEA (68 μL, 0.39 mmol). This mixture was stirred at RT for 15 min, then 2-dimethylaminoethanol (20 μL, 0.19 mmol) was added. The reaction was stirred at RT for 1 h, then it was diluted with EtOAc and with sat. aqueous NaHCO₃ solution (3×) and brine, filtered through a phase separator and concentrated under vacuum. The crude material was purified by FC on Biotage silica-NH gel (from 5% to 46% of EtOAc in c-Hex as eluent), affording the title compound (37 mg, 0.08 mmol, 63% yield) as pale yellow solid.

LC-MS (ESI): m/z (M+2): 229.2 (Method 1)

¹H NMR (400 MHz, DMSO-d₆) δ ppm 9.06-9.11 (m, 1H), 9.04 (d, J=2.0 Hz, 1H), 8.45 (s, 1H), 7.95 (dd, J=6.8, 2.6 Hz, 1H), 7.42-7.48 (m, 1H), 7.26-7.38 (m, 2H), 7.10 (s, 1H), 4.42 (t, J=5.7 Hz, 2H), 4.21 (br d, J=3.9 Hz, 2H), 3.51 (br s, 2H), 2.65 (t, J=5.6 Hz, 2H), 2.23 (s, 6H).

### Example 25

### 3-[6-(5-chloro-2-fluorophenyl)-2H,3H,4H-pyrido[3,2-b][1,4]oxazin-8-yl]-5-methoxypyridine

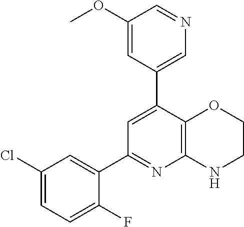

In a suitable, a mixture of Intermediate 46 (150 mg, 0.50 mmol), 5-methoxypyridine-3-boronic acid (92 mg, 0.60 mmol), Na₂CO₃ (106 mg, 1 mmol) and PdCl₂(dtbpf) (49 mg, 0.08 mmol) was dissolved in 1,4-dioxane/water (6.3 mL, 3:1 ratio). The vessel was sealed, evacuated and backfilled with N₂ (3×) and heated at 60° C. in a PLS, for 10 hrs. The reaction was diluted with EtOAc, filtered through a Celite® pad, washing with EtOAc. The filtrate was washed with brine. The organic phase was filtered through a phase separator and evaporated under vacuum. The crude material was purified by FC on Biotage silica gel (from 15 to 60% of EtOAc in c-Hex as eluent), affording the title compound (86 mg, 0.23 mmol, 46% yield) as white solid.

LC-MS (ESI): m/z (M+1): 372.2 (Method 2)

¹H NMR (500 MHz, DMSO-d₆) δ ppm 8.39 (d, J=1.6 Hz, 1H), 8.31 (d, J=3.0 Hz, 1H), 7.94 (dd, J=6.9, 2.7 Hz, 1H), 7.55 (dd, J=2.7, 1.6 Hz, 1H), 7.41-7.48 (m, 1H), 7.34 (dd, J=11.0, 8.8 Hz, 1H), 7.22 (s, 1H), 7.06 (d, J=1.6 Hz, 1H), 4.20 (t, J=4.4 Hz, 2H), 3.88 (s, 3H), 3.46-3.54 (m, 2H).

### Example 26

### 5-[6-(5-chloro-2-fluorophenyl)-2H,3H,4H-pyrido[3,2-b][1,4]oxazin-8-yl]-N-[2-(pyrrolidin-1-yl)ethyl]pyridine-3-carboxamide

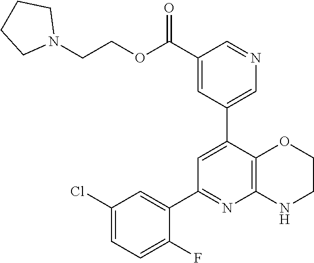

Prepared from Intermediate 32 (50 mg, 0.13 mmol) and 1-(2-aminoethyl)pyrrolidine (25 μL, 0.19 mmol), following similar procedure as for Example 11. The title compound (33 mg, 0.07 mmol, 53% yield) was obtained as pale yellow solid.

LC-MS (ESI): m/z (M+2): 241.8 (Method 1)

¹H NMR (400 MHz, DMSO-d₆) δ ppm 8.98 (d, J=2.2 Hz, 1H), 8.92 (d, J=2.2 Hz, 1H), 8.71 (t, J=5.5 Hz, 1H), 8.34 (t, J=2.1 Hz, 1H), 7.94 (dd, J=6.8, 2.6 Hz, 1H), 7.41-7.49 (m, 1H), 7.34 (dd, J=11.0, 8.8 Hz, 1H), 7.27 (br s, 1H), 7.10 (d, J=1.5 Hz, 1H), 4.21 (t, J=4.2 Hz, 2H), 3.51 (br d, J=2.2 Hz, 2H), 3.41 (q, J=6.7 Hz, 2H), 2.58 (t, J=6.9 Hz, 2H), 2.42-2.50 (m, 4H), 1.62-1.73 (m, 4H).

### Example 27

### [3-({5-[6-(5-chloro-2-fluorophenyl)-2H,3H,4H-pyrido[3,2-b][1,4]oxazin-8-yl]pyridin-3-yl}oxy)propyl]dimethylamine

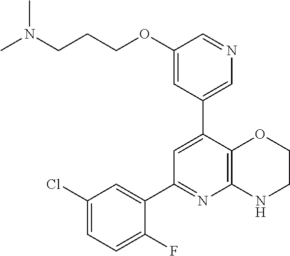

Example 27 was prepared starting from Intermediate 46 (100 mg, 0.33 mmol) and Intermediate 56 (0.50 theoretical mmol), following similar procedure as for Example 25. Title compound (65 mg, 0.15 mmol, 44% yield) was obtained as pale yellow glassy solid.

LC-MS (ESI): m/z (M+1): 222.2 (Method 1)

¹H NMR (600 MHz, DMSO-d₆) δ ppm 8.37 (d, J=1.6 Hz, 1H), 8.29 (d, J=2.6 Hz, 1H), 7.94 (dd, J=6.8, 2.8 Hz, 1H), 7.51-7.57 (m, 1H), 7.41-7.49 (m, 1H), 7.33 (dd, J=10.9, 8.9 Hz, 1H), 7.21 (s, 1H), 7.06 (d, J=1.3 Hz, 1H), 4.20 (t, J=4.4 Hz, 2H), 4.13 (t, J=6.4 Hz, 2H), 3.45-3.53 (m, 2H), 2.36 (t, J=7.1 Hz, 2H), 2.14 (s, 6H), 1.87 (quin, J=6.8 Hz, 2H).

### Example 28

### 5-[6-(5-chloro-2-fluorophenyl)-2H,3H,4H-pyrido[3,2-b][1,4]oxazin-8-yl]-N-[3-(piperazin-1-yl)propyl]pyridine-3-carboxamide

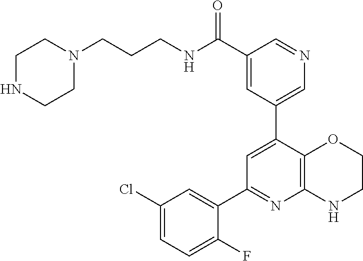

Prepared from Intermediate 57 (50 mg, 0.09 mmol), following the procedure described for Example 16. Title compound (37.8 mg, 0.07 mmol, 73% yield) was obtained as pale yellow solid.

LC-MS (ESI): m/z (M+2): 256.3 (Method 1)

¹H NMR (600 MHz, DMSO-d₆) δ ppm 8.98 (d, J=2.0 Hz, 1H), 8.92 (d, J=2.0 Hz, 1H), 8.73 (t, J=5.4 Hz, 1H), 8.33 (t, J=2.0 Hz, 1H), 7.94 (dd, J=6.8, 2.8 Hz, 1H), 7.43-7.48 (m, 1H), 7.34 (dd, J=10.9, 8.9 Hz, 1H), 7.26 (s, 1H), 7.10 (d, J=1.0 Hz, 1H), 4.21 (t, J=4.4 Hz, 2H), 3.48-3.53 (m, 2H), 3.28-3.35 (m, 2H), 2.66 (t, J=4.8 Hz, 4H), 2.22-2.35 (m, 6H), 1.81-2.17 (m, 1H), 1.69 (quin, J=7.1 Hz, 2H).

### Example 29

### 5-[6-(5-chloro-2-fluorophenyl)-2H,3H,4H-pyrido[3,2-b][1,4]oxazin-8-yl]pyridin-3-ol

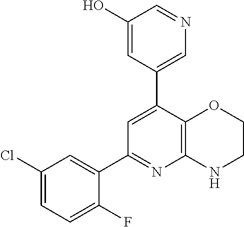

Example 29 was prepared starting from Intermediate 46 (150 mg, 0.5 mmol) and 5-(4,4,5,5-tetramethyl-1,3,2-dioxaborolan-2-yl)pyridin-3-ol (133 mg, 0.6 mmol), following similar procedure as for Example 17. Title compound (27 mg, 0.08 mmol, 15% yield) was obtained as white solid.

LC-MS (ESI): m/z (M+2): 179.6 (Method 1)

¹H NMR (500 MHz, DMSO-d₆) δ ppm 10.16 (br s, 1H), 8.23 (d, J=1.9 Hz, 1H), 8.13 (d, J=2.7 Hz, 1H), 7.95 (dd, J=6.7, 2.9 Hz, 1H), 7.40-7.50 (m, 1H), 7.36 (dd, J=2.6, 1.8 Hz, 1H), 7.33 (dd, J=11.0, 8.8 Hz, 1H), 7.21 (s, 1H), 7.02 (d, J=1.6 Hz, 1H), 4.19 (t, J=4.4 Hz, 2H), 3.43-3.56 (m, 2H).

### Example 30

### 5-[6-(5-chloro-2-fluorophenyl)-2H,3H,4H-pyrido[3,2-b][1,4]oxazin-8-yl]-N-[3-(dimethylamino)propyl]pyridin-3-amine

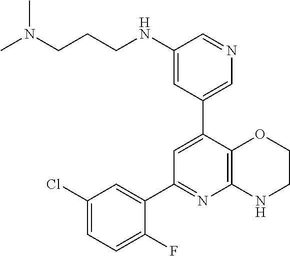

Example 30 was prepared starting from Intermediate 46 (60 mg, 0.2 mmol) and Intermediate 59 (0.35 theoretical mmol), following similar procedure as for Example 25. Title compound (20 mg, 0.05 mmol, 22% yield) was obtained as pale yellow glassy solid.

LC-MS (ESI): m/z (M+2): 221.7 (Method 1)

¹H NMR (400 MHz, DMSO-d₆) δ ppm 7.91-7.99 (m, 3H), 7.40-7.48 (m, 1H), 7.29-7.37 (m, 1H), 7.16 (s, 1H), 7.05 (s, 1H), 7.00 (d, J=1.1 Hz, 1H), 5.97 (t, J=5.5 Hz, 1H), 4.17 (t, J=4.3 Hz, 2H), 3.49 (br d, J=0.9 Hz, 2H), 3.08 (q, J=6.6 Hz, 2H), 2.29 (t, J=7.0 Hz, 2H), 2.13 (s, 6H), 1.68 (quin, J=6.9 Hz, 2H).

### Example 31

### N-{5-[6-(5-chloro-2-fluorophenyl)-2H,3H,4H-pyrido[3,2-b][1,4]oxazin-8-yl]pyridin-3-yl}-4-(methylamino)butanamide

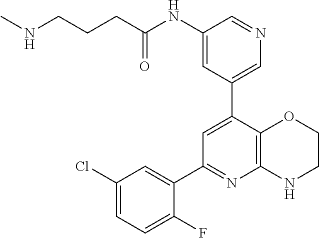

Prepared from Intermediate 63 (93 mg, 0.17 mmol), following the procedure described for Example 16. Title compound (60 mg, 0.13 mmol, 79% yield) was obtained as pale yellow solid.

LC-MS (ESI): m/z (M+2): 228.7 (Method 1)

¹H NMR (400 MHz, DMSO-d₆) δ ppm 10.06-10.54 (m, 1H), 8.78 (d, J=2.4 Hz, 1H), 8.46 (d, J=2.0 Hz, 1H), 8.23 (t, J=2.1 Hz, 1H), 7.96 (dd, J=6.9, 2.7 Hz, 1H), 7.45 (ddd, J=8.7, 4.0, 2.9 Hz, 1H), 7.34 (dd, J=11.0, 8.8 Hz, 1H), 7.25 (s, 1H), 7.03 (d, J=1.5 Hz, 1H), 4.20 (t, J=4.2 Hz, 2H), 3.45-3.55 (m, 2H), 2.45-2.53 (m, 2H), 2.40 (t, J=7.5 Hz, 2H), 2.26 (s, 3H), 1.72 (quin, J=7.1 Hz, 3H).

### Example 32

### 5-[6-(5-chloro-2-fluorophenyl)-2H,3H,4H-pyrido[3,2-b][1,4]oxazin-8-yl]-N-[3-(methylamino)propyl]pyridine-3-carboxamide

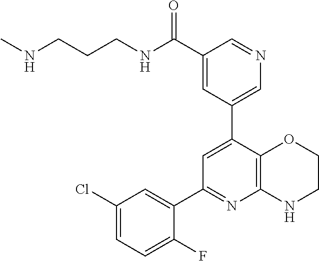

Prepared from Intermediate 64 (84 mg, 0.15 mmol), following the procedure described for Example 16. Title compound (58 mg, 0.13 mmol, 84% yield) was obtained as pale yellow solid.

LC-MS (ESI): m/z (M+2): 228.7 (Method 1)

¹H NMR (400 MHz, DMSO-d₆) δ ppm 8.98 (d, J=2.2 Hz, 1H), 8.92 (d, J=2.0 Hz, 1H), 8.79 (t, J=5.4 Hz, 1H), 8.33 (t, J=2.1 Hz, 1H), 7.94 (dd, J=6.8, 2.9 Hz, 1H), 7.45 (ddd, J=8.7, 4.0, 3.1 Hz, 1H), 7.34 (dd, J=11.0, 9.0 Hz, 1H), 7.27 (s, 1H), 7.10 (d, J=1.3 Hz, 1H), 4.21 (t, J=4.3 Hz, 2H), 3.48-3.54 (m, 2H), 3.21-3.44 (m, 2H), 2.46-2.52 (m, 2H), 2.26 (s, 3H), 1.66 (quin, J=6.8 Hz, 2H), 1.56-2.01 (m, 1H).

### Example 33

### N-{5-[6-(5-chloro-2-fluorophenyl)-2H,3H,4H-pyrido[3,2-b][1,4]oxazin-8-yl]pyridin-3-yl}-4-(piperazin-1-yl)butanamide

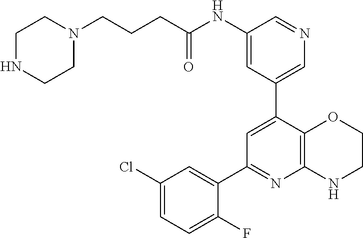 [SMILES: O=C(CCCN1CCNCC1)Nc1cncc(-c2cc(-c3cc(Cl)ccc3F)nc3c2OCCN3)c1]

Prepared from Intermediate 67 (86 mg, 0.14 mmol), following the procedure described for Example 16. Title compound (58.5 mg, 0.11 mmol, 81% yield) was obtained as pale yellow solid.

LC-MS (ESI): m/z (M+2): 256.3 (Method 1)

¹H NMR (400 MHz, DMSO-d₆) δ ppm 10.18 (s, 1H), 8.78 (d, J 2.4 Hz, 1H), 8.46 (d, J=1.8 Hz, 1H), 8.23 (t, J=2.2 Hz, 1H), 7.96 (dd, J=6.8, 2.6 Hz, 1H), 7.41-7.48 (m, 1H), 7.34 (dd, J=11.1, 8.9 Hz, 1H), 7.25 (s, 1H), 7.03 (d, J=1.5 Hz, 1H), 4.20 (t, J=4.3 Hz, 2H), 3.44-3.57 (m, 2H), 2.62-2.69 (m, 4H), 2.37 (t, J=7.3 Hz, 2H), 1.88-2.33 (m, 7H), 1.74 (quin, J=7.1 Hz, 2H).

### Example 34

### 3-[6-(5-chloro-2-fluorophenyl)-2H,3H,4H-pyrido[3,2-b][1,4]oxazin-8-yl]-5-[(oxolan-2-yl)methoxy]pyridine

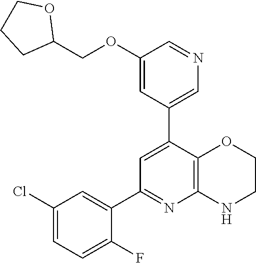 [SMILES: Fc1ccc(Cl)cc1-c1cc(-c2cncc(OCC3CCCO3)c2)c2c(n1)NCCO2]

Example 34 was prepared starting from Intermediate 24 (68 mg, 0.20 mmol) and 3-[(oxolan-2-yl)methoxy]-5-(4,4,5,5-tetramethyl-1,3,2-dioxaborolan-2-yl)pyridine (Intermediate 69, 0.30 theoretical mmol), following similar procedure as for Example 5. Title compound (43 mg, 0.10 mmol, 50% yield) was obtained as pale yellow solid.

LC-MS (ESI): m/z (M+1): 442.3 (Method 1)

¹H NMR (400 MHz, DMSO-d₆) δ ppm 8.38 (d, J=1.5 Hz, 1H), 8.31 (d, J=2.9 Hz, 1H), 7.93 (dd, J=6.8, 2.9 Hz, 1H), 7.56 (dd, J=2.6, 1.8 Hz, 1H), 7.41-7.48 (m, 1H), 7.33 (dd, J=11.0, 8.8 Hz, 1H), 7.21 (s, 1H), 7.06 (d, J=1.8 Hz, 1H), 4.02-4.25 (m, 5H), 3.75-3.83 (m, 1H), 3.64-3.73 (m, 1H), 3.49 (br d, J=2.4 Hz, 2H), 1.61-2.13 (m, 4H).

### Example 35

### 2-methyl-6-(8-{1H-pyrazolo[3,4-b]pyridin-5-yl}-2H,3H,4H-pyrido[3,2-b][1,4]oxazin-6-yl)pyridine

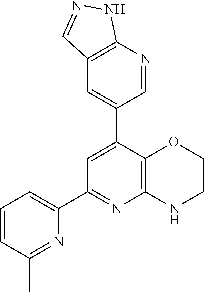 [SMILES: Cc1cccc(-c2cc(-c3cnc4[nH]ncc4c3)c3c(n2)NCCO3)n1]

Prepared from Intermediate 75 (61 mg, 0.14 mmol), following the procedure described for Example 16. Title compound (40 mg, 0.12 mmol, 85% yield) was obtained as yellow solid.

LC-MS (ESI): m/z (M+1): 345.37 (Method 1)

¹H NMR (500 MHz, DMSO-d₆) δ ppm 13.76 (br s, 1H), 8.74 (d, J=2.2 Hz, 1H), 8.45 (d, J=2.2 Hz, 1H), 8.22 (s, 1H), 8.01 (d, J=7.7 Hz, 1H), 7.75 (t, J=7.7 Hz, 1H), 7.67 (s, 1H), 7.19 (d, J=7.4 Hz, 1H), 7.02 (s, 1H), 4.21 (t, J=4.4 Hz, 2H), 3.41-3.59 (m, 2H), 2.50 (s, 3H).

### Example 36

### 3-[6-(5-chloro-2-fluorophenyl)-2H,3H,4H-pyrido[3,2-b][1,4]oxazin-8-yl]-5-(oxolan-3-yloxy)pyridine

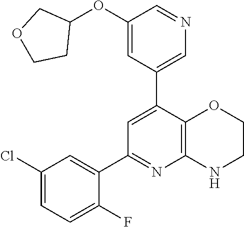 [SMILES: Fc1ccc(Cl)cc1-c1cc(-c2cncc(OC3CCOC3)c2)c2c(n1)NCCO2]

Example 36 was prepared starting from Intermediate 24 (68 mg, 0.20 mmol) and 3-(oxolan-3-yloxy)-5-(4,4,5,5-tetramethyl-1,3,2-dioxaborolan-2-yl)pyridine (Intermediate 77, 0.30 theoretical mmol), following similar procedure as for Example 5. Title compound (60 mg, 0.14 mmol, 70% yield) was obtained as white solid.

LC-MS (ESI): m/z (M+1): 428.3 (Method 1)

¹H NMR (400 MHz, DMSO-d₆) δ ppm 8.40 (d, J 1.6 Hz, 1H), 8.29 (d, J 2.7 Hz, 1H), 7.94 (dd, J=6.8, 2.9 Hz, 1H), 7.52-7.60 (m, 1H), 7.40-7.48 (m, 1H), 7.34 (dd, J=11.0, 8.8 Hz, 1H), 7.22 (s, 1H), 7.07 (d, J=1.3 Hz, 1H), 5.11-5.27 (m, 1H), 4.21 (t, J=4.2 Hz, 2H), 3.71-3.95 (m, 4H), 3.49 (br d, J=2.9 Hz, 2H), 2.17-2.32 (m, 1H), 1.88-2.11 (m, 1H).

### Example 37

### 5-[6-(5-chloro-2-fluorophenyl)-2H,3H,4H-pyrido[3,2-b][1,4]oxazin-8-yl]-N-[2-(4-methylpiperazin-1-yl)ethyl]pyridine-3-carboxamide

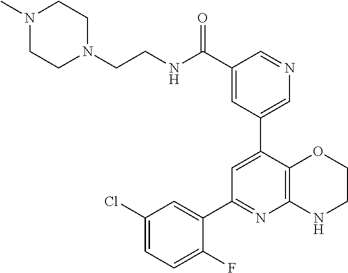 [SMILES: CN1CCN(CCNC(=O)c2cncc(-c3cc(-c4cc(Cl)ccc4F)nc4c3OCCN4)c2)CC1]

Prepared from Intermediate 32 (50 mg, 0.13 mmol) and 2-(4-methylpiperazin-1-yl)ethan-1-amine (29 μL, 0.19 mmol), following similar procedure as for Example 11. The title compound (27 mg, 0.05 mmol, 41% yield) was obtained as pale yellow solid.

LC-MS (ESI): m/z (M+2): 256.3 (Method 1)

¹H NMR (400 MHz, DMSO-d₆) δ ppm 8.97 (d, J=2.0 Hz, 1H), 8.93 (d, J=2.0 Hz, 1H), 8.66 (t, J=5.6 Hz, 1H), 8.33 (t, J=2.0 Hz, 1H), 7.94 (dd, J=6.7, 2.8 Hz, 1H), 7.41-7.50 (m, 1H), 7.34 (dd, J=10.9, 8.8 Hz, 1H), 7.27 (s, 1H), 7.10 (d, J=1.3 Hz, 1H), 4.21 (t, J=4.1 Hz, 2H), 3.51 (br d, J=2.3 Hz, 2H), 3.40 (q, J=6.5 Hz, 2H), 2.45-2.49 (m, 2H), 2.20-2.49 (m, 8H), 2.13 (s, 3H).

### Example 38

### N-{5-[6-(5-chloro-2-fluorophenyl)-2H,3H,4H-pyrido[3,2-b][1,4]oxazin-8-yl]pyridin-3-yl}-3-(4-methylpiperazin-1-yl)propanamide

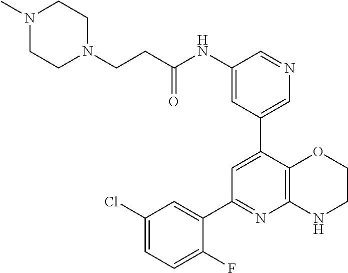 [SMILES: CN1CCN(CCC(=O)Nc2cncc(-c3cc(-c4cc(Cl)ccc4F)nc4c3OCCN4)c2)CC1]

Example 38 was prepared starting from Intermediate 24 (60 mg, 0.17 mmol) and 3-(4-methylpiperazin-1-yl)-N-[5-(4,4,5,5-tetramethyl-1,3,2-dioxaborolan-2-yl)pyridin-3-yl]propanamide (Intermediate 79, 0.26 theoretical mmol), following similar procedure as for Example 5. Title compound (60 mg, 0.12 mmol, 67% yield) as ivory solid.

LC-MS (ESI): m/z (M+2): 256.3 (Method 1)

¹H NMR (400 MHz, DMSO-d₆) δ ppm 10.42 (s, 1H), 8.77 (d, J=2.0 Hz, 1H), 8.48 (d, J=1.6 Hz, 1H), 8.21 (s, 1H), 7.95 (dd, J=6.8, 2.7 Hz, 1H), 7.39-7.50 (m, 1H), 7.30-7.38 (m, 1H), 7.25 (s, 1H), 7.03 (s, 1H), 4.20 (br t, J=3.8 Hz, 2H), 3.50 (br s, 2H), 2.59-2.71 (m, 2H), 2.20-2.57 (m, 10H), 2.14 (s, 3H).

### Example 39

### Methyl 5-[6-(6-methylpyridin-2-yl)-2H,3H,4H-pyrido[3,2-b][1,4]oxazin-8-yl]pyridine-3-carboxylate

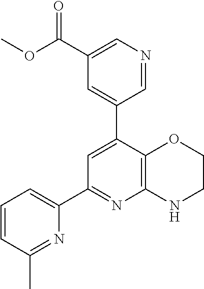 [SMILES: COC(=O)c1cncc(-c2cc(-c3cccc(C)n3)nc3c2OCCN3)c1]

Prepared from Intermediate 80 (99 mg, 0.22 mmol), following the procedure described for Example 16. Title compound (76 mg, 0.21 mmol, 98% yield) was obtained as yellow solid.

LC-MS (ESI): m/z (M+1): 363.3 (Method 1)

¹H NMR (400 MHz, DMSO-d₆) δ ppm 9.10 (d, J 2.0 Hz, 1H), 9.05 (d, J 2.2 Hz, 1H), 8.44 (t, J=2.1 Hz, 1H), 8.00 (d, J=7.9 Hz, 1H), 7.76 (t, J=7.7 Hz, 1H), 7.62 (s, 1H), 7.20 (d, J=7.5 Hz, 1H), 7.12 (s, 1H), 4.22 (t, J=4.3 Hz, 2H), 3.93 (s, 3H), 3.51 (br d, J=2.4 Hz, 2H), 2.51 (br s, 3H).

### Example 40

### Cyclopropyl 5-[6-(5-chloro-2-fluorophenyl)-2H,3H,4H-pyrido[3,2-b][1,4]oxazin-8-yl]pyridine-3-carboxylate

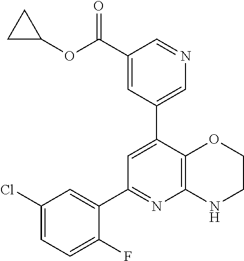 [SMILES: O=C(OC1CC1)c1cncc(-c2cc(-c3cc(Cl)ccc3F)nc3c2OCCN3)c1]

Prepared from Intermediate 32 (63 mg, 0.16 mmol) and cyclopropanol (103 μL, 1.63 mmol), following the procedure described for Example 24. The title compound (40 mg, 0.09 mmol, 58% yield) was obtained as white solid.

LC-MS (ESI): m/z (M+1): 426.3 (Method 1)

¹H NMR (500 MHz, DMSO-d₆) δ ppm 9.05 (d, J=1.9 Hz, 1H), 9.03 (d, J=2.2 Hz, 1H), 8.41 (t, J=2.1 Hz, 1H), 7.94 (dd, J=6.6, 2.7 Hz, 1H), 7.45 (ddd, J=8.8, 3.8, 3.0 Hz, 1H), 7.34 (dd, J=11.0, 8.8 Hz, 1H), 7.29 (br s, 1H), 7.09 (d, J=1.1 Hz, 1H), 4.37 (tt, J=6.2, 3.2 Hz, 1H), 4.21 (br t, J=4.1 Hz, 2H), 3.51 (br d, J=1.6 Hz, 2H), 0.60-1.01 (m, 4H).

### Example 41

### 3-[6-(5-chloro-2-fluorophenyl)-2H,3H,4H-pyrido[3,2-b][1,4]oxazin-8-yl]-5-[(1-methylpiperidin-4-yl)oxy]pyridine

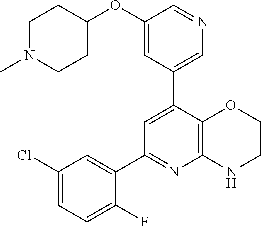 [SMILES: CN1CCC(Oc2cncc(-c3cc(-c4cc(Cl)ccc4F)nc4c3OCCN4)c2)CC1]

Example 41 was prepared starting from Intermediate 24 (60 mg, 0.17 mmol) and 3-[(1-methylpiperidin-4-yl)oxy]-5-(4,4,5,5-tetramethyl-1,3,2-dioxaborolan-2-yl)pyridine (Intermediate 86, 0.26 theoretical mmol), following similar procedure as for Example 5. Title compound (55 mg, 0.12 mmol, 69% yield) was obtained as white solid

LC-MS (ESI): m/z (M+2): 228.3 (Method 1)

¹H NMR (400 MHz, DMSO-d₆) δ ppm 8.35 (d, J=1.6 Hz, 1H), 8.30 (d, J=3.0 Hz, 1H), 7.94 (dd, J=6.9, 2.7 Hz, 1H), 7.57 (dd, J=2.6, 1.8 Hz, 1H), 7.40-7.48 (m, 1H), 7.34 (dd, J=11.0, 8.8 Hz, 1H), 7.21 (s, 1H), 7.05 (d, J=1.6 Hz, 1H), 4.52 (tt, J=8.1, 3.8 Hz, 1H), 4.20 (t, J=4.4 Hz, 2H), 3.44-3.58 (m, 2H), 2.56-2.70 (m, 2H), 2.17 (s, 5H), 1.96 (br dd, J=10.0, 3.2 Hz, 2H), 1.60-1.74 (m, 2H).

### Example 42

### 3-[6-(5-chloro-2-fluorophenyl)-2H,3H,4H-pyrido[3,2-b][1,4]oxazin-8-yl]-5-[(1-methylpiperidin-4-yl)methoxy]pyridine

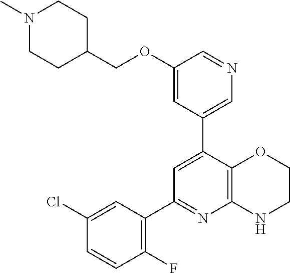 [SMILES: CN1CCC(COc2cncc(-c3cc(-c4cc(Cl)ccc4F)nc4c3OCCN4)c2)CC1]

Example 42 was prepared starting from Intermediate 24 (60 mg, 0.17 mmol) and 3-[(1-methylpiperidin-4-yl)methoxy]-5-(4,4,5,5-tetramethyl-1,3,2-dioxaborolan-2-yl)pyridine (Intermediate 88, 0.26 theoretical mmol), following similar procedure as for Example 5. Title compound (51 mg, 0.11 mmol, 62% yield) was obtained as white solid.

LC-MS (ESI): m/z (M+2): 235.3 (Method 1)

¹H NMR (400 MHz, DMSO-d₆) δ ppm 8.37 (d, J 1.8 Hz, 1H), 8.29 (d, J 2.6 Hz, 1H), 7.93 (dd, J=6.8, 2.9 Hz, 1H), 7.50-7.58 (m, 1H), 7.40-7.49 (m, 1H), 7.33 (dd, J=11.0, 8.8 Hz, 1H), 7.21 (s, 1H), 7.05 (d, J=1.5 Hz, 1H), 4.20 (t, J=4.1 Hz, 2H), 3.95 (d, J=6.1 Hz, 2H), 3.49 (br d, J=2.9 Hz, 2H), 2.78 (br d, J=11.4 Hz, 2H), 2.15 (s, 3H), 1.80-1.91 (m, 2H), 1.63-1.78 (m, 3H), 1.22-1.39 (m, 2H).

### Example 43

### 3-[bis(2-hydroxyethyl)amino]-N-{5-[6-(5-chloro-2-fluorophenyl)-2H,3H,4H-pyrido[3,2-b][1,4]oxazin-8-yl]pyridin-3-yl}propanamide

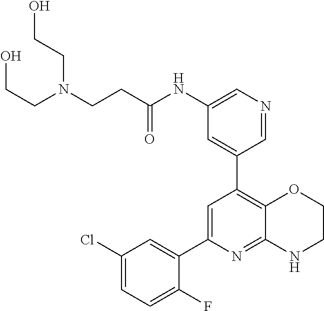 [SMILES: O=C(CCN(CCO)CCO)Nc1cncc(-c2cc(-c3cc(Cl)ccc3F)nc3c2OCCN3)c1]

Prepared from Intermediate 92 (43 mg, 0.07 mmol), following the procedure described for Example 16. Title compound (30 mg, 0.06 mmol, 83% yield) was obtained as white solid.

LC-MS (ESI): m/z (M+2): 258.8 (Method 1)

¹H NMR (400 MHz, DMSO-d₆) δ ppm 10.56 (s, 1H), 8.77 (d, J=2.2 Hz, 1H), 8.46 (d, J=1.8 Hz, 1H), 8.24 (s, 1H), 7.95 (dd, J=6.6, 2.6 Hz, 1H), 7.40-7.51 (m, 1H), 7.34 (dd, J=11.2, 9.0 Hz, 1H), 7.24 (s, 1H), 7.03 (d, J=0.9 Hz, 1H), 4.43 (t, J=5.3 Hz, 2H), 4.20 (br t, J=3.9 Hz, 2H), 3.42-3.54 (m, 6H), 2.84 (t, J=6.6 Hz, 2H), 2.58 (t, J=6.1 Hz, 4H), 2.45-2.52 (m, 2H).

### Example 44

### N-{5-[6-(5-chloro-2-fluorophenyl)-2H,3H,4H-pyrido[3,2-b][1,4]oxazin-8-yl]pyridin-3-yl}-3-[(2-hydroxyethyl)(methyl)amino]propanamide

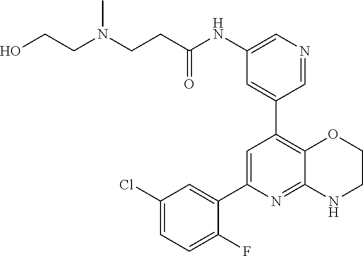 [SMILES: CN(CCO)CCC(=O)Nc1cncc(-c2cc(-c3cc(Cl)ccc3F)nc3c2OCCN3)c1]

Prepared from Intermediate 93 (73.3 mg, 0.11 mmol), following the procedure described for Example 16. Title compound (44 mg, 0.09 mmol, 80% yield) as pale yellow solid.

LC-MS (ESI): m/z (M+2): 243.6 (Method 1)

¹H NMR (500 MHz, DMSO-d₆) δ ppm 10.54 (s, 1H), 8.77 (d, J=2.3 Hz, 1H), 8.46 (d, J=1.9 Hz, 1H), 8.24 (t, J=2.1 Hz, 1H), 7.95 (dd, J=6.9, 2.9 Hz, 1H), 7.40-7.50 (m, 1H), 7.34 (dd, J=11.1, 8.8 Hz, 1H), 7.25 (s, 1H), 7.03 (d, J=1.4 Hz, 1H), 4.44 (t, J=5.3 Hz, 1H), 4.20 (t, J=4.3 Hz, 2H), 3.49 (q, J=6.0 Hz, 4H), 2.70 (t, J=6.8 Hz, 2H), 2.48-2.52 (m, 2H), 2.46 (t, J=6.1 Hz, 2H), 2.23 (s, 3H).

### Example 45

### N-{5-[6-(5-chloro-2-fluorophenyl)-2H,3H,4H-pyrido[3,2-b][1,4]oxazin-8-yl]pyridin-3-yl}-3-[methyl(oxetan-3-yl)amino]propenamide

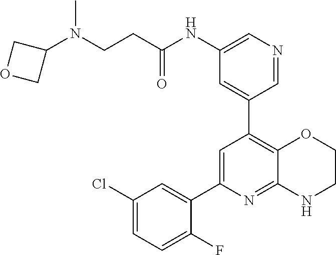 [SMILES: CN(CCC(=O)Nc1cncc(-c2cc(-c3cc(Cl)ccc3F)nc3c2OCCN3)c1)C1COC1]

Prepared from Intermediate 94 (53 mg, 0.09 mmol), following the procedure described for Example 16. Title compound (37 mg, 0.07 mmol, 84% yield) was obtained as pale yellow solid.

LC-MS (ESI): m/z (M+2): 249.7 (Method 1)

¹H NMR (500 MHz, DMSO-d₆) δ ppm 10.33 (s, 1H), 8.79 (br d, J=2.1 Hz, 1H), 8.48 (d, J=1.4 Hz, 1H), 8.23 (br s, 1H), 7.96 (br dd, J=6.7, 2.6 Hz, 1H), 7.40-7.54 (m, 1H), 7.29-7.40 (m, 1H), 7.25 (br s, 1H), 7.03 (s, 1H), 4.52 (t, J=6.0 Hz, 2H), 4.41 (br t, J=6.0 Hz, 2H), 4.20 (br t, J=4.0 Hz, 2H), 3.44-3.61 (m, 3H), 2.52-2.58 (m, 4H), 2.09 (s, 3H).

### Example 46

### N-{5-[6-(5-chloro-2-fluorophenyl)-2H,3H,4H-pyrido[3,2-b][1,4]oxazin-8-yl]pyridin-3-yl}-3-[methyl(oxetan-3-yl)amino]propanamide

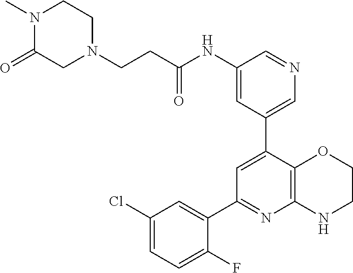 [SMILES: CN1CCN(CCC(=O)Nc2cncc(-c3cc(-c4cc(Cl)ccc4F)nc4c3OCCN4)c2)CC1=O]

Prepared from Intermediate 95 (60 mg, 0.10 mmol), following the procedure described for Example 16. Title compound (44 mg, 0.08 mmol, 87% yield) was obtained as pale yellow solid.

LC-MS (ESI): m/z (M+2): 263.2 (Method 1)

¹H NMR (400 MHz, DMSO-d₆) δ ppm 10.28 (s, 1H), 8.78 (d, J=2.4 Hz, 1H), 8.48 (d, J=1.8 Hz, 1H), 8.22 (t, J=2.1 Hz, 1H), 7.96 (dd, J=6.8, 2.9 Hz, 1H), 7.45 (dt, J=8.6, 3.5 Hz, 1H), 7.34 (dd, J=11.0, 8.8 Hz, 1H), 7.25 (s, 1H), 7.03 (d, J=1.3 Hz, 1H), 4.20 (t, J=4.1 Hz, 2H), 3.50 (br s, 2H), 3.25 (t, J=5.4 Hz, 2H), 3.03 (s, 2H), 2.80 (s, 3H), 2.64-2.74 (m, 4H), 2.51-2.60 (m, 2H).

### Example 47

### N-{5-[6-(5-chloro-2-fluorophenyl)-2H,3H,4H-pyrido[3,2-b][1,4]oxazin-8-yl]pyridin-3-yl}-3-(4-methyl-2-oxopiperazin-1-yl)propanamide

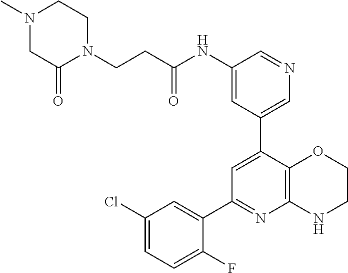 [SMILES: CN1CCN(CCC(=O)Nc2cncc(-c3cc(-c4cc(Cl)ccc4F)nc4c3OCCN4)c2)C(=O)C1]

Prepared from Intermediate 96 (30 mg, 0.05 mmol), following the procedure described for Example 16. Title compound (20 mg, 0.04 mmol, 79% yield) was obtained as pale yellow solid.

LC-MS (ESI): m/z (M+2): 263.3 (Method 1)

¹H NMR (400 MHz, DMSO-d₆) δ ppm 10.34 (s, 1H), 8.77 (d, J=2.2 Hz, 1H), 8.49 (d, J=1.8 Hz, 1H), 8.21 (s, 1H), 7.95 (dd, J=6.7, 2.8 Hz, 1H), 7.45 (dt, J=8.6, 3.5 Hz, 1H), 7.34 (dd, J=11.0, 8.9 Hz, 1H), 7.25 (s, 1H), 7.03 (d, J=1.1 Hz, 1H), 4.20 (br t, J=4.0 Hz, 2H), 3.57 (t, J=7.0 Hz, 2H), 3.50 (br s, 2H), 3.29-3.35 (m, 2H), 2.91 (s, 2H), 2.61 (t, J=7.0 Hz, 2H), 2.54 (t, J=5.4 Hz, 2H), 2.19 (s, 3H).

### Example 48

### Methyl 2-{[2-({5-[6-(5-chloro-2-fluorophenyl)-2H,3H,4H-pyrido[3,2-b][1,4]oxazin-8-yl]pyridin-3-yl}carbamoyl)ethyl](methyl)amino}acetate

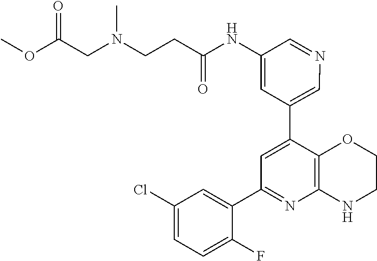 [SMILES: COC(=O)CN(C)CCC(=O)Nc1cncc(-c2cc(-c3cc(Cl)ccc3F)nc3c2OCCN3)c1]

Prepared from Intermediate 97 (60 mg, 0.10 mmol), following the procedure described for Example 16. Title compound (44 mg, 0.09 mmol, 88% yield) was obtained as pale yellow solid.

LC-MS (ESI): m/z (M+2): 257.6 (Method 1)

¹H NMR (400 MHz, DMSO-d₆) δ ppm 10.43 (s, 1H), 8.78 (d, J=2.2 Hz, 1H), 8.48 (d, J=1.5 Hz, 1H), 8.23 (s, 1H), 7.95 (dd, J=6.8, 2.6 Hz, 1H), 7.41-7.49 (m, 1H), 7.30-7.38 (m, 1H), 7.25 (br s, 1H), 7.03 (s, 1H), 4.20 (br t, J=3.9 Hz, 2H), 3.61 (s, 3H), 3.50 (br s, 2H), 3.34 (s, 2H), 2.82 (t, J=6.9 Hz, 2H), 2.50 (br s, 2H), 2.32 (s, 3H).

### Comparative Newly Synthesised Compounds Having a Phenyl Ring, Instead of a Pyridyl Ring or a Pyridinyl Ring Fused to a 5 or 6-Membered Heteroaryl Ring, Linked to the Pyrido Oxazine Bicycle

### Example C1: 6-(5-chloro-2-fluorophenyl)-8-(4-methoxyphenyl)-2H,3H,4H-pyrido[3,2-b][1,4]oxazine

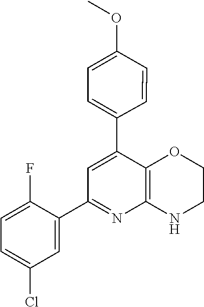 [SMILES: COc1ccc(-c2cc(-c3cc(Cl)ccc3F)nc3c2OCCN3)cc1]

Title compound was prepared starting from Intermediate 24 (50 mg, 0.15 mmol) and 4-methoxyphenylboronic acid (26.5 mg, 0.17 mmol) following similar procedure as for Example 5. Title compound (40 mg, 0.11 mmol, 74% yield) was obtained as white solid.

LC-MS (ESI): m/z (M+1): 371.2 (Method 1)

¹H NMR (500 MHz, DMSO-d₆) δ ppm 7.92-7.97 (m, 1H), 7.53-7.57 (m, 2H), 7.40-7.46 (m, 1H), 7.32 (dd, J=11.3, 8.8 Hz, 1H), 7.08 (s, 1H), 6.99-7.04 (m, 3H), 4.17 (t, J=4.5 Hz, 2H), 3.80 (s, 3H), 3.44-3.54 (m, 2H).

### Example C2: 4-[6-(5-chloro-2-fluorophenyl)-2H,3H,4H-pyrido[3,2-b][1,4]oxazin-8-yl]phenol

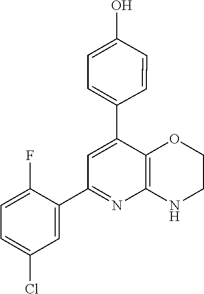 [SMILES: Oc1ccc(-c2cc(-c3cc(Cl)ccc3F)nc3c2OCCN3)cc1]

Title compound was prepared starting from Intermediate 24 (60 mg, 0.17 mmol) and 4-hydroxyphenylboronic acid (28.9 mg, 021 mmol) following similar procedure as for Example 5. Title compound (50 mg, 0.14 mmol, 80% yield) was obtained as white solid.

LC-MS (ESI): m/z (M+1): 357.2 (Method 1)

¹H NMR (500 MHz, DMSO-d₆) δ ppm 9.66 (br s, 1H), 7.94 (dd, J=6.9, 2.7 Hz, 1H), 7.38-7.51 (m, 3H), 7.32 (dd, J=11.1, 8.9 Hz, 1H), 7.04 (br s, 1H), 6.99 (d, J=1.4 Hz, 1H), 6.83 (d, J=8.5 Hz, 2H), 4.16 (br t, J=4.1 Hz, 2H), 3.48 (br s, 2H).

Pharmacological Activity of the Compounds of the Invention

In Vitro Assay

The enzymatic activity of compounds of the present invention was monitored measuring the formation of ADP using the ADP-GLO Kinases assay. Following the incubation of the purified enzyme, a substrate and ATP, the produced ADP was converted into ATP, which in turn was converted into light by Ultra-Glo Luciferase. The luminescent signal positively correlated with ADP amount and kinase activity. Briefly, the kinase reaction was performed by incubating 2.6 nM of the purified, commercially available human ALK5 (recombinant TGF 31 N-term GST-tagged, 80-end), a final concentration of TGFβ1 peptide 94.5 μM (Promega, T36-58) and ultra-pure ATP (Promega V915B). The ATP concentration was set at the Km value (concentration of substrate which permits the enzyme to achieve half maximal velocity (Vmax)) of ALK5 (5 μM). All reactions/incubations were performed at 25° C. Compound and ALK5 kinase were mixed and incubated for 15 mins. Reactions were initiated by addition of ATP at a final concentration in the assay of 0.83p M. After an incubation of 150 min, the reaction was stopped, and ADP production detected with ADP-Glo kit according to manufacturer's indications. The assay was performed in 384-well format and was validated using a selection of reference compounds that was tested in 11 point concentration-response curve.

The results for individual compounds are provided below in Table 12 wherein the compounds are classified in term of potency (nM) with respect to their inhibitory activity on ALK5 receptor.

TABLE 12

| | ALK5 IC50 |
|---|---|
| Example No | (nM) |
| 8, 10, 12, 17, 18, 21, 22, 25, 27, 31, 41, 44 | ≤1 |
| 2, 3, 4, 5, 6, 9, 16, 19, 20, 23, 24, 28, 29, 30, | 1-5 |
| 32, 33, 36, 37, 38, 40, 42, 43, 45 | |
| 1, 7, 11, 13, 14, 15, 26, 34, 35, 39, 46, 47, 48 | 5-10 |

As it can be appreciated, all the compounds of Table 12 show a good activity as antagonists of ALK5 receptor.

### Comparative Examples

Compounds of the examples C1 and C2 were tested in the same in vitro assay described above.

TABLE 13

| Example No | ALK5 IC50 (nM) |
|---|---|
| C1 | 10000 |
| C2 | 1710 |

The compounds of the present invention, as shown in Table 12, have a potency even lower than 1 nM, whereas comparative examples C1 and C2 have a potency higher than 1700 nM and even largely higher.

These data demonstrate that, conversely to the compounds C1 and C2 characterized by a phenyl ring linked to the pyrido oxazine bicycle, in the compounds of the present invention, characterized by a pyridinyl ring or a pyridinyl ring fused to a 5 or 6-membered heteroaryl ring, the presence of the pyridinyl ring or of a pyridinyl ring fused to a 5 or 6-membered heteroaryl ring, unexpectedly and remarkably determines a relevant increase in the inhibitory activity on the ALK5 receptor.

1: A compound of formula (I) 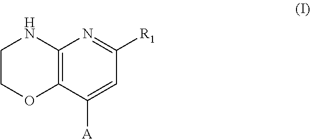
wherein
R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;
A is selected from the group consisting of A₁, A₂, A₃, A₄ and A₅ 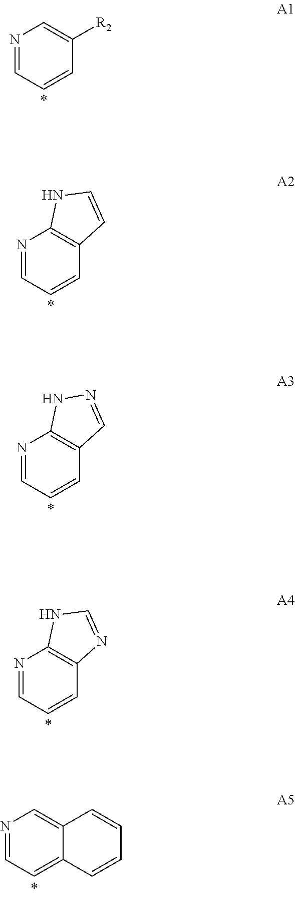
R₂ is H or is selected from the group consisting of —NR₃C(O)R₄, —C(O)NR₃R₄, —C(O)OR₅, NR₃R₄ and —OR₆;
R₃ is H or —(C₁—C₆)alkyl;
R₄ is H or is selected from the group consisting of —(C₁-C₆)alkylene-NR_{A}R_{B} and —(C₁-C₆)alkylene-heterocycloalkyl, wherein said heterocycloalkyl is optionally substituted by one or more groups selected from oxo and —(C₁-C₆)alkyl;
R₅ is selected from the group consisting of —(C₁-C₆)alkyl, —(C₁-C₆)alkylene-NR_{A}R_{B} and cycloalkyl;
R₆ is H or is selected from the group consisting of —(C₁-C₆)alkyl, heterocycloalkyl, —(C₁-C₆)alkylene-NR_{A}R_{B}, —(C₁-C₆)alkylene-heterocycloalkyl, wherein said heterocycloalkyl is optionally substituted by one or more —(C₁-C₆)alkyl;
R_{A} is H or is selected from the group consisting of —(C₁-C₆)alkyl and —(C₁-C₆)hydroxyalkyl;
R_{B} is selected from the group consisting of —(C₁-C₆)alkyl, heterocycloalkyl, —(C₁-C₆)alkylene-C(O)O—(C₁-C₆)alkyl and —(C₁-C₆)hydroxyalkyl;
or a pharmaceutically acceptable salt of said compound.

2: The compound of formula (I) or pharmaceutically acceptable salt thereof according to claim 1, wherein A is group A1 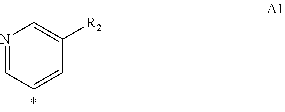
represented by the formula (Ia) 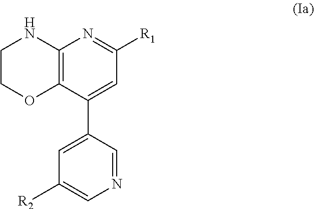
wherein
R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms;
R₂ is H or is selected from the group consisting of —NR₃C(O)R₄, —C(O)NR₃R₄, —C(O)OR₅ and —OR₆;
R₃ is H or —(C₁-C₆)alkyl;
R₄ is selected from the group consisting of —(C₁-C₆)alkylene-NR_{A}R_{B} and —(C₁-C₆)alkylene-heterocycloalkyl;
R₅ is selected from the group consisting of —(C₁-C₆)alkyl, —(C₁-C₆)alkylene-NR_{A}R_{B} and cycloalkyl;
R₆ is H or is selected from the group consisting of —(C₁-C₆)alkyl, heterocycloalkyl, —(C₁-C₆)alkylene-NR_{A}R_{B}, —(C₁-C₆)alkylene-heterocycloalkyl, wherein said heterocycloalkyl is optionally substituted by one or more —(C₁-C₆)alkyl;
R_{A} is H or is selected from the group consisting of —(C₁-C₆)alkyl and —(C₁-C₆)hydroxyalkyl; and
R_{B} is selected from the group consisting of —(C₁-C₆)alkyl, —(C₁-C₆)alkylene-C(O)O—(C₁-C₆)alkyl and —(C₁-C₆)hydroxyalkyl.

3: The compound or pharmaceutically acceptable salt thereof according to claim 2, wherein the compound is selected from the group consisting of:
3-[6-(5-chloro-2-fluorophenyl)-2H,3H,4H-pyrido[3,2-b][1,4]oxazin-8-yl]pyridine;
5-[6-(5-chloro-2-fluorophenyl)-2H,3H,4H-pyrido[3,2-b][1,4]oxazin-8-yl]pyridin-3-amine;
5-[6-(5-chloro-2-fluorophenyl)-2H,3H,4H-pyrido[3,2-b][1,4]oxazin-8-yl]pyridine-3-carboxamide;
5-[6-(5-chloro-2-fluorophenyl)-2H,3H,4H-pyrido[3,2-b][1,4]oxazin-8-yl]-N-methylpyridine-3-carboxamide;
N-{5-[6-(5-chloro-2-fluorophenyl)-2H,3H,4H-pyrido[3,2-b][1,4]oxazin-8-yl]pyridin-2-yl}-3-(dimethylamino)propenamide;
N-{5-[6-(5-chloro-2-fluorophenyl)-2H,3H,4H-pyrido[3,2-b][1,4]oxazin-8-yl]pyridin-3-yl}-3-(morpholin-4-yl)propenamide;
5-[6-(5-chloro-2-fluorophenyl)-2H,3H,4H-pyrido[3,2-b][1,4]oxazin-8-yl]-N-[2-(morpholin-4-yl)ethyl]pyridine-3-carboxamide;
N-{5-[6-(5-chloro-2-fluorophenyl)-2H,3H,4H-pyrido[3,2-b][1,4]oxazin-8-yl]pyridin-3-yl}-3-(pyrrolidin-1-yl)propenamide;
N-{5-[6-(5-chloro-2-fluorophenyl)-2H,3H,4H-pyrido[3,2-b][1,4]oxazin-8-yl]pyridin-3-yl}-2-(dimethylamino)acetamide;
N-{5-[6-(5-chloro-2-fluorophenyl)-2H,3H,4H-pyrido[3,2-b][1,4]oxazin-8-yl]pyridin-3-yl}-3-(piperazin-1-yl)propenamide;
N-{5-[6-(5-chloro-2-fluorophenyl)-2H,3H,4H-pyrido[3,2-b][1,4]oxazin-8-yl]pyridin-3-yl}-4-(dimethylamino)butanamide;
N-{5-[6-(5-chloro-2-fluorophenyl)-2H,3H,4H-pyrido[3,2-b][1,4]oxazin-8-yl]pyridin-3-yl}-3-(piperidin-1-yl)propenamide;
methyl 5-[6-(5-chloro-2-fluorophenyl)-2H,3H,4H-pyrido[3,2-b][1,4]oxazin-8-yl]pyridine-3-carboxylate;
5-[6-(5-chloro-2-fluorophenyl)-2H,3H,4H-pyrido[3,2-b][1,4]oxazin-8-yl]-N-[2-(piperazin-1-yl)ethyl]pyridine-3-carboxamide;
5-[6-(5-chloro-2-fluorophenyl)-2H,3H,4H-pyrido[3,2-b][1,4]oxazin-8-yl]-N-[3-(dimethylamino)propyl]pyridine-3-carboxamide;
N-{5-[6-(5-chloro-2-fluorophenyl)-2H,3H,4H-pyrido[3,2-b][1,4]oxazin-8-yl]pyridin-3-yl}-3-(methylamino)propenamide;
5-[6-(5-chloro-2-fluorophenyl)-2H,3H,4H-pyrido[3,2-b][1,4]oxazin-8-yl]-N-[2-(methylamino)ethyl]pyridine-3-carboxamide;
2-(dimethylamino)ethyl 5-[6-(5-chloro-2-fluorophenyl)-2H,3H,4H-pyrido[3,2-b][1,4]oxazin-8-yl]pyridine-3-carboxylate;
3-[6-(5-chloro-2-fluorophenyl)-2H,3H,4H-pyrido[3,2-b][1,4]oxazin-8-yl]-5-methoxypyridine;
5-[6-(5-chloro-2-fluorophenyl)-2H,3H,4H-pyrido[3,2-b][1,4]oxazin-8-yl]-N-[2-(pyrrolidin-1-yl)ethyl]pyridine-3-carboxamide;
[3-({5-[6-(5-chloro-2-fluorophenyl)-2H,3H,4H-pyrido[3,2-b][1,4]oxazin-8-yl]pyridin-3-yl}oxy)propyl]dimethylamine;
5-[6-(5-chloro-2-fluorophenyl)-2H,3H,4H-pyrido[3,2-b][1,4]oxazin-8-yl]-N-[3-(piperazin-1-yl)propyl]pyridine-3-carboxamide;
5-[6-(5-chloro-2-fluorophenyl)-2H,3H,4H-pyrido[3,2-b][1,4]oxazin-8-yl]pyridin-3-ol;
5-[6-(5-chloro-2-fluorophenyl)-2H,3H,4H-pyrido[3,2-b][1,4]oxazin-8-yl]-N-[3-(dimethylamino)propyl]pyridin-3-amine;
N-{5-[6-(5-chloro-2-fluorophenyl)-2H,3H,4H-pyrido[3,2-b][1,4]oxazin-8-yl]pyridin-3-yl}-4-(methylamino)butanamide;
5-[6-(5-chloro-2-fluorophenyl)-2H,3H,4H-pyrido[3,2-b][1,4]oxazin-8-yl]-N-[3-(methylamino)propyl]pyridine-3-carboxamide;
N-{5-[6-(5-chloro-2-fluorophenyl)-2H,3H,4H-pyrido[3,2-b][1,4]oxazin-8-yl]pyridin-3-yl}-4-(piperazin-1-yl)butanamide;
3-[6-(5-chloro-2-fluorophenyl)-2H,3H,4H-pyrido[3,2-b][1,4]oxazin-8-yl]-5-[(oxolan-2-yl)methoxy]pyridine;
3-[6-(5-chloro-2-fluorophenyl)-2H,3H,4H-pyrido[3,2-b][1,4]oxazin-8-yl]-5-(oxolan-3-yloxy)pyridine;
5-[6-(5-chloro-2-fluorophenyl)-2H,3H,4H-pyrido[3,2-b][1,4]oxazin-8-yl]-N-[2-(4-methylpiperazin-1-yl)ethyl]pyridine-3-carboxamide;
N-{5-[6-(5-chloro-2-fluorophenyl)-2H,3H,4H-pyrido[3,2-b][1,4]oxazin-8-yl]pyridin-3-yl}-3-(4-methylpiperazin-1-yl)propenamide;
methyl 5-[6-(6-methylpyridin-2-yl)-2H,3H,4H-pyrido[3,2-b][1,4]oxazin-8-yl]pyridine-3-carboxylate;
cyclopropyl 5-[6-(5-chloro-2-fluorophenyl)-2H,3H,4H-pyrido[3,2-b][1,4]oxazin-8-yl]pyridine-3-carboxylate;
3-[6-(5-chloro-2-fluorophenyl)-2H,3H,4H-pyrido[3,2-b][1,4]oxazin-8-yl]-5-[(1-methylpiperidin-4-yl)oxy]pyridine;
3-[6-(5-chloro-2-fluorophenyl)-2H,3H,4H-pyrido[3,2-b][1,4]oxazin-8-yl]-5-[(1-methylpiperidin-4-yl)methoxy]pyridine;
3-[bis(2-hydroxyethyl)amino]-N-{5-[6-(5-chloro-2-fluorophenyl)-2H,3H,4H-pyrido[3,2-b][1,4]oxazin-8-yl]pyridin-3-yl}propenamide;
N-{5-[6-(5-chloro-2-fluorophenyl)-2H,3H,4H-pyrido[3,2-b][1,4]oxazin-8-yl]pyridin-3-yl}-3-[(2-hydroxyethyl)(methyl)amino]propenamide;
N-{5-[6-(5-chloro-2-fluorophenyl)-2H,3H,4H-pyrido[3,2-b][1,4]oxazin-8-yl]pyridin-3-yl}-3-[methyl(oxetan-3-yl)amino]propenamide;
N-{5-[6-(5-chloro-2-fluorophenyl)-2H,3H,4H-pyrido[3,2-b][1,4]oxazin-8-yl]pyridin-3-yl}-3-(4-methyl-3-oxopiperazin-1-yl)propenamide;
N-{5-[6-(5-chloro-2-fluorophenyl)-2H,3H,4H-pyrido[3,2-b][1,4]oxazin-8-yl]pyridin-3-yl}-3-(4-methyl-2-oxopiperazin-1-yl)propenamide; and
methyl 2-{[2-({5-[6-(5-chloro-2-fluorophenyl)-2H,3H,4H-pyrido[3,2-b][1,4]oxazin-8-yl]pyridin-3-yl}carbamoyl)ethyl](methyl)amino}acetate.

4: The compound of formula (I) or pharmaceutically acceptable salt thereof according to claim 1, wherein A is group A2 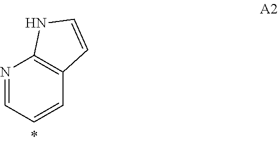
represented by the formula (Ib) 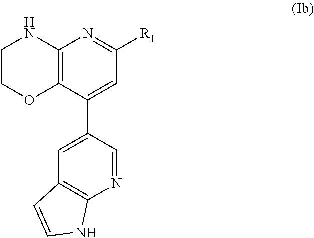
wherein R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms.

5: The compound of formula (I) or pharmaceutically acceptable salt thereof according to claim 1, wherein A is group A3 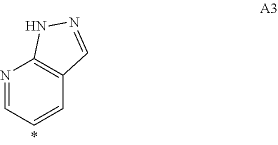
represented by the formula (Ic) 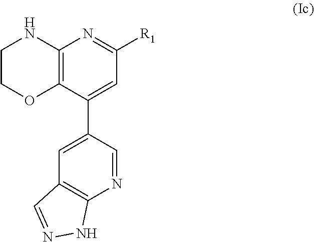
wherein R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms.

6: The compound or pharmaceutically acceptable salt thereof according to claim 5, wherein the compound selected from the group consisting of:
6-(5-chloro-2-fluorophenyl)-8-{1H-pyrazolo[3,4-b]pyridin-5-yl}-2H,3H,4H-pyrido[3,2-b][1,4]oxazine; and
2-methyl-6-(8-{1H-pyrazolo[3,4-b]pyridin-5-yl}-2H,3H,4H-pyrido[3,2-b][1,4]oxazin-6-yl)pyridine.

7: The compound of formula (I) or pharmaceutically acceptable salt thereof according to claim 1, wherein A is group A4 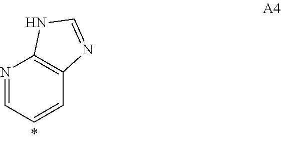
represented by the formula (Id) 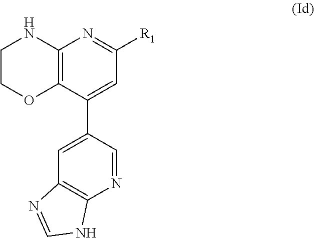
wherein R₁ is aryl optionally substituted by one or more groups selected from halogen atoms and —(C₁-C₆)alkyl; or R₁ is heteroaryl containing one or more heteroatoms selected from N, O and S, wherein said heteroaryl is optionally substituted by one or more groups selected from —(C₁-C₆)alkyl and halogen atoms.

8: The compound of formula (I) or pharmaceutically acceptable salt thereof according to claim 1, wherein A is group A5 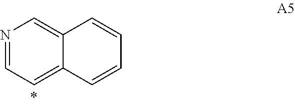
represented by the formula (Ie) 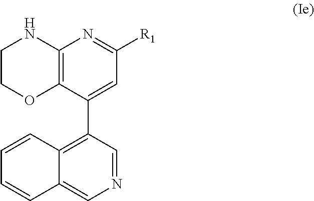
wherein R₁ is aryl optionally substituted by one or more groups selected from halogen atoms and —(C₁-C₆)alkyl: or R₁ is heteroaryl containing one or more heteroatoms selected from N, O and S, wherein said heteroaryl is optionally substituted by one or more groups selected from —(C₁-C₆)alkyl and halogen atoms.

9. (canceled)

10: A pharmaceutical composition comprising the compound of formula (I) or pharmaceutically acceptable salt thereof according to claim 1, in admixture with one or more pharmaceutically acceptable carriers or excipients.

11: The pharmaceutical composition according to claim 9, which is formulated for administration by inhalation.

12. (canceled)

13: A c method of treating a disease, disorder or condition mediated by ALK5 signaling pathway in a mammal, comprising administering to a mammal in need thereof the pharmaceutical composition of claim 10.

14: A method for treating fibrosis and/or a disease, disorder or condition that involves fibrosis, comprising administering to a subject in need thereof the pharmaceutical composition of claim 10.

15: The method according to claim 14, wherein the fibrosis and/or disease, disorder or condition that involves fibrosis is at least one selected from the group consisting of pulmonary fibrosis, idiopathic pulmonary fibrosis (IPF), hepatic fibrosis, renal fibrosis, ocular fibrosis, cardiac fibrosis, arterial fibrosis and systemic sclerosis.

16: The method according to claim 15, wherein the fibrosis is idiopathic pulmonary fibrosis (IPF).
